SEQUENCE LISTING (43 sequences)

SEQ ID NO: 1 - protein, 32 aa, Artificial sequence
  VelA coiled-coil
VAQLEEKVKTLRAENYELKSEVQRLEEQVAQL

SEQ ID NO: 2 - protein, 32 aa, Artificial sequence
  VelB coiled-coil
VDELQAEVDQLEDENYALKTKVAQLRKKVEKL

SEQ ID NO: 3 - protein, 47 aa, Artificial sequence
  VelA-IPV
GASTTVAQLEEKVKTLRAENYELKSEVQRLEEQVAQLGSIPVSLRSG

SEQ ID NO: 4 - protein, 47 aa, Artificial sequence
  VelB-IPV
GASTSVDELQAEVDQLEDENYALKTKVAQLRKKVEKLGSIPVSLRSG

SEQ ID NO: 5 - protein, 5 aa, Artificial sequence
  Exemplary linker
GSGGS

SEQ ID NO: 6 - protein, 4 aa, Artificial sequence
  Exemplary linker
GGGS

SEQ ID NO: 7 - protein, 12 aa, Artificial sequence
  Exemplary linker
SGGGGGGGGGGS

SEQ ID NO: 8 - protein, 4 aa, Artificial sequence
  Exemplary linker
GGAA

SEQ ID NO: 9 - protein, 5 aa, Artificial sequence
  Exemplary linker
GGGGS

SEQ ID NO: 10 - protein, 5 aa, Artificial sequence
  Exemplary linker
LAAAA

SEQ ID NO: 11 - protein, 4 aa, Artificial sequence
  Exemplary linker
GGSG

SEQ ID NO: 12 - protein, 5 aa, Artificial sequence
  Exemplary linker
GGSGG

SEQ ID NO: 13 - protein, 5 aa, Artificial sequence
  Exemplary linker
GSGSG

SEQ ID NO: 14 - protein, 5 aa, Artificial sequence
  Exemplary linker
GSGGG

SEQ ID NO: 15 - protein, 5 aa, Artificial sequence
  Exemplary linker
GGGSG

SEQ ID NO: 16 - protein, 5 aa, Artificial sequence
  Exemplary linker
GSSSG

SEQ ID NO: 17 - protein, 6 aa, Artificial sequence
  Exemplary protease site
  misc_feature (4)..(6): Xaa can be any naturally occurring amino acid
PLGXXX

SEQ ID NO: 18 - protein, 5 aa, Artificial sequence
  Exemplary protease site
PLGVR

SEQ ID NO: 19 - protein, 8 aa, Artificial sequence
  Exemplary protease site
IPVSLRSG

SEQ ID NO: 20 - protein, 8 aa, Artificial sequence
  Exemplary protease site
LSGRSDNY

SEQ ID NO: 21 - protein, 6 aa, Artificial sequence
  Exemplary protease site
GPLGVR

SEQ ID NO: 22 - protein, 118 aa, Artificial sequence
  Heavy chain variable sequence of hB6H12.3 and hB6H12.3 (deamidation mutant)
EVQLLESGGGLVQPGGSLRLSCAASGFTFSGYGMSWVRQAPGKRLEWVATITSGGTYTYYPDSVKGRFTISRDNSKNTLYLQMNSLRAEDTAIYFCARSLAGNAMDYWGQGTLVTVSS

SEQ ID NO: 23 - protein, 108 aa, Artificial sequence
  Light chain variable sequence of hB6H12.3
EIVMTQSPDFQSVTPKEKVTLTCRASQTISDYLHWYQQKPDQSPKLLIKFASQSISGVPSRFSGSGSGSDFTLTINSLEAEDAATYYCQNGHGFPRTFGQGTKLEIKR

SEQ ID NO: 24 - protein, 108 aa, Artificial sequence
  Light chain variable sequence of hB6H12.3 (deamidation mutant)
EIVMTQSPDFQSVTPKEKVTLTCRASQTISDYLHWYQQKPDQSPKLLIKFASQSISGVPSRFSGSGSGSDFTLTINSLEAEDAATYYCQNAHGFPRTFGQGTKLEIKR

SEQ ID NO: 25 - protein, 5 aa, Artificial sequence
  hvH1 HCDR1 of hB6H12.3 and hB6H12.3 (deamidation mutant) (Kabat)
GYGMS

SEQ ID NO: 26 - protein, 17 aa, Artificial sequence
  hvH1 HCDR2 of hB6H12.3 and hB6H12.3 (deamidation mutant) (Kabat)
TITSGGTYTYYPDSVKG

SEQ ID NO: 27 - protein, 9 aa, Artificial sequence
  hvH1 HCDR3 of hB6H12.3 and hB6H12.3 (deamidation mutant) (Kabat)
SLAGNAMDY

SEQ ID NO: 28 - protein, 8 aa, Artificial sequence
  hvH1 HCDR1 of hB6H12.3 and hB6H12.3 (deamidation mutant) (IMGT)
GFTFSGYG

SEQ ID NO: 29 - protein, 8 aa, Artificial sequence
  hvH1 HCDR2 of hB6H12.3 and hB6H12.3 (deamidation mutant) (IMGT)
ITSGGTYT

SEQ ID NO: 30 - protein, 11 aa, Artificial sequence
  hvH1 HCDR3 of hB6H12.3 and hB6H12.3 (deamidation mutant) (IMGT)
ARSLAGNAMDY

SEQ ID NO: 31 - protein, 11 aa, Artificial sequence
  hvK3 LCDR1 of hB6H12.3 and hB6H12.3 (deamidation mutant) (Kabat)
RASQTISDYLH

SEQ ID NO: 32 - protein, 7 aa, Artificial sequence
  hvK3 LCDR2 of hB6H12.3 and hB6H12.3 (deamidation mutant) (Kabat)
FASQSIS

SEQ ID NO: 33 - protein, 9 aa, Artificial sequence
  hvK3 LCDR3 of hB6H12.3 and hB6H12.3 (deamidation mutant) (Kabat)
QNGHGFPRT

SEQ ID NO: 34 - protein, 9 aa, Artificial sequence
  hvK3 (G91A) LCDR3 of hB6H12.3 and hB6H12.3 (deamidation mutant) (Kabat)
QNAHGFPRT

SEQ ID NO: 35 - protein, 6 aa, Artificial sequence
  hvK3 LCDR1 of hB6H12.3 and hB6H12.3 (deamidation mutant) (IMGT)
QTISDY

SEQ ID NO: 36 - protein, 3 aa, Artificial sequence
  hvK3 LCDR2 of hB6H12.3 and hB6H12.3 (deamidation mutant) (IMGT)
FAS

SEQ ID NO: 37 - protein, 9 aa, Artificial sequence
  hvK3 LCDR3 of hB6H12.3 and hB6H12.3 (deamidation mutant) (IMGT)
QNGHGFPRT

SEQ ID NO: 38 - protein, 9 aa, Artificial sequence
  hvK3 (G91A) LCDR3 of hB6H12.3 and hB6H12.3 (deamidation mutant) (IMGT)
QNAHGFPRT

SEQ ID NO: 39 - protein, 488 aa, Artificial sequence
  Complete Heavy Chain version 1 of a masked anti-CD47 antibody
QGASTSVDELQAEVDQLEDENYALKTKVAQLRKKVEKLGSIPVSLRSGEVQLLESGGGLVQPGGSLRLSCAASGFTFSGYGMSWVRQAPGKRLEWVATITSGGTYTYYPDSVKGRFTISRDNSKNTLYLQMNSLRAEDTAIYFCARSLAGNAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQK

SEQ ID NO: 40 - protein, 487 aa, Artificial sequence
  Complete Heavy Chain version 2 of a masked anti-CD47 antibody
QGASTSVDELQAEVDQLEDENYALKTKVAQLRKKVEKLGSIPVSLRSGEVQLLESGGGLVQPGGSLRLSCAASGFTFSGYGMSWVRQAPGKRLEWVATITSGGTYTYYPDSVKGRFTISRDNSKNTLYLQMNSLRAEDTAIYFCARSLAGNAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQ

SEQ ID NO: 41 - protein, 40 aa, Artificial sequence
  Heavy Chain masking sequence
QGASTSVDELQAEVDQLEDENYALKTKVAQLRKKVEKLGS

SEQ ID NO: 42 - protein, 262 aa, Artificial sequence
  Complete Light Chain of a masked anti-CD47 antibody
QGASTTVAQLEEKVKTLRAENYELKSEVQRLEEQVAQLGSIPVSLRSGEIVMTQSPDFQSVTPKEKVTLTCRASQTISDYLHWYQQKPDQSPKLLIKFASQSISGVPSRFSGSGSGSDFTLTINSLEAEDAATYYCQNGHGFPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 40 aa, Artificial sequence
  Light Chain masking sequence
QGASTTVAQLEEKVKTLRAENYELKSEVQRLEEQVAQLGS

(19) United States
(12) Patent Application Publication
(10) Pub. No.: US 2022/0233709 A1
(43) Pub. Date: Jul. 28, 2022

(54) Masked Antibody Formulations

(71) Applicant: Seagen Inc., Bothell, WA (US)

(72) Inventors: Danielle Leiske, Bothell, WA (US); Elise Cunningham, Bothell, WA (US); Shan Jiang, Bothell, WA (US); Lori Westendorf, Bothell, WA (US); Michael Feldhaus, Boston, MA (US); Eoin Francis James Cosgrave, Mill Creek, WA (US); Catherine Marie Eakin, Bothell, WA (US)

(73) Assignee: Seagen Inc., Bothell, WA (US)

(21) Appl. No.: 17/614,788

(22) PCT Filed: Jun. 4, 2020
(86) PCT No.: PCT/US2020/036035

§ 371 Date: Nov. 29, 2021

Related U.S. Application Data
(60) Provisional application No. 62/906,862, filed on Sep. 27, 2019.
(60) Provisional application No. 62/857,364, filed on Jun. 5, 2019.

(51) Int. Cl. A61K 47/68; A61K 47/65; A61K 9/19; A61K 9/08; A61P 35/00

(52) U.S. Cl. CPC A61K 47/6817; A61K 47/6849; A61K 47/65; A61P 35/00; A61K 9/19; A61K 9/08; A61K 47/6803

(57) ABSTRACT

Formulations comprising masked antibodies are provided. In some embodiments, there is reduced aggregation of the masked antibodies in the formulations. In various embodiments, the formulations are pharmaceutical formulations suitable for use in therapeutic treatment.

### CROSS-REFERENCE TO RELATED APPLICATIONS

This application claims the benefit of priority of U.S. Provisional Application No. 62/857,364, filed Jun. 5, 2019, and U.S. Provisional Application No. 62/906,862, filed Sep. 27, 2019, each of which is incorporated by reference herein in its entirety for any purpose.

### FIELD OF THE INVENTION

The present invention relates to the field of antibody formulations. In particular, the present invention relates to formulations of masked antibodies with reduced aggregation. In some embodiments, the masked antibodies comprise anti-CD47 antibodies.

### BACKGROUND

Current antibody-based therapeutics may have less than optimal selectivity for the intended target. Although monoclonal antibodies are typically specific for binding to their intended targets, most target molecules are not specific to the disease site and may be present in cells or tissues other than the disease site.

Several approaches have been described for overcoming these off-target effects by engineering antibodies to have a cleavable linker attached to an inhibitory or masking domain that inhibits antibody binding (see, e.g., WO2003/068934, WO2004/009638, WO 2009/025846, WO2101/081173 and WO2014103973). The linker can be designed to be cleaved by enzymes that are specific to certain tissues or pathologies, thus enabling the antibody to be preferentially activated in desired locations. Masking moieties can act by binding directly to the binding site of an antibody or can act indirectly via steric hindrance. Various masking moieties, linkers, protease sites and formats of assembly have been proposed. The extent of masking may vary between different formats as may the compatibility of masking moieties with expression, purification, conjugation, or pharmacokinetics of antibodies.

The present invention relates to formulations of masked antibodies with reduced aggregation. In some embodiments, the masked antibodies comprise a first coiled-coil domain linked to a heavy chain variable region of the antibody and a second coiled-coil domain linked to a light chain variable region of the antibody. The presence of these potentially hydrophobic coiled-coil polypeptide sequences can lead to aggregation during storage. In some embodiments, the present formulations may result in reduced aggregation of the masked antibodies.

### SUMMARY

The present disclosure addresses formulating masked antibodies that comprise a removable masking agent (e.g., a coiled coil masking agent) that prevents binding of the antibodies to their intended targets until the masking agent is cleaved off or otherwise removed. In other words, the masking agent masks the antigen binding portion of the antibody so that it cannot interact with its targets. In certain therapeutic uses, the masking agent can be removed (e.g., cleaved) by one or more molecules (e.g., proteases) that are present in an in vivo environment after administration of the masked antibody to a patient. In other, for example non-therapeutic, uses, a masking agent could be removed by adding one or more proteases to the medium in which the antibody is being used. Removal of the masking agent restores the ability of the antibodies to bind to their targets, thus enabling specific targeting of the antibodies. In some embodiments herein, the antibodies are CD47 antibodies.

The presence of coiled coil masking agents, for example, could increase the chances of aggregation of the antibodies during storage prior to use. Thus, the present disclosure addresses formulations of masked antibodies that may reduce aggregation of the masked antibodies during storage.

In some embodiments, an aqueous formulation is provided, wherein the aqueous formulation comprises a masked antibody comprising a first masking domain comprising a first coiled-coil domain, wherein the first masking domain is linked to a heavy chain variable region of an antibody and a second masking domain comprising a second coiled-coil domain, wherein the second masking domain is linked to a light chain variable region of the antibody, wherein the first coiled-coil domain comprises the sequence VDELQAEVDQLEDENYALKTKVAQLRKKVEKL (SEQ ID NO: 2), and the second coiled-coil domain comprises the sequence VAQLEEKVKTLRAENYELKSEVQRLEEQVAQL (SEQ ID NO: 1), and wherein the formulation comprises a buffer, and wherein the pH of the formulation is from 3.5 to 4.5.

In some embodiments, an aqueous formulation is provided, wherein the aqueous formulation comprises a masked antibody comprising a first masking domain comprising a first coiled-coil domain, wherein the first masking domain is linked to a heavy chain variable region of an antibody and a second masking domain comprising a second coiled-coil domain, wherein the second masking domain is linked to a light chain variable region of the antibody, wherein the the first coiled-coil domain comprises the sequence VAQLEEKVKTLRAENYELKSEVQRLEEQVAQL (SEQ ID NO: 1), and the second coiled-coil domain comprises the sequence VDELQAEVDQLEDENYALKTKVAQLRKKVEKL (SEQ ID NO: 2), and wherein the formulation comprises a buffer, and wherein the pH of the formulation is from 3.5 to 4.5.

In some embodiments, the buffer is selected from acetate, succinate, lactate, and glutamate. In some embodiments, the concentration of the buffer is from 10 mM to 100 mM, or from 10 mM to 80 mM, or from 10 mM to 70 mM, or from 10 mM to 60 mM, or from 10 mM to 50 mM, or from 10 mM to 40 mM, or from 20 mM to 100 mM, or from 20 mM to 80 mM, or from 20 mM to 70 mM, or from 20 mM to 60 mM, or from 20 mM to 50 mM, or from 20 mM to 40 mM.

In some embodiments, the formulation comprises at least one cryoprotectant. In some embodiments, at least one cryoprotectant is selected from sucrose, trehalose, mannitol, and glycine. In some embodiments, the total cryoprotectant concentration in the aqueous formulation is 6-12% w/v.

In some embodiments, the formulation comprises sucrose or trehalose. In some embodiments, the formulation comprises mannitol and trehalose, or glycine and trehalose.

In some embodiments, the formulation comprises at least one excipient selected from glycerol, polyethylene glycol (PEG), hydroxypropyl beta-cyclodextrin (HPBCD), polysorbate 20 (PS20), polysorbate 80 (PS80), and poloxamer 188 (P188).

In some embodiments, the formulation does not comprise added salt. In some embodiments, the formulation does not comprise added NaCl, KCl, or MgCl2.

In some embodiments, the concentration of the masked antibody in the formulation is from 1 to 30 mg/mL, or from 5 to 30 mg/mL, or from 10 to 30 mg/mL, or from 5 to 25 mg/mL, or from 5 to 20 mg/mL, or from 10 to 20 mg/mL, or from 10 to 25 mg/mL, or from 15 to 25 mg/mL.

In some embodiments, the formulation comprises 40 mM acetate, 8% sucrose, 0.05% PS80, pH 3.7-4.4. In some embodiments, the formulation comprises 20 mg/mL or 18 mg/mL masked antibody.

In some embodiments, the formulation comprises 40 mM glutamate, 8% w/v trehalose dihydrate, and 0.05% polysorbate 80, pH 3.6-4.2. In some embodiments, the formulation comprises 20 mg/mL or 18 mg/mL masked antibody.

In some embodiments, each masking domain comprises a protease-cleavable linker and is linked to the heavy chain or light chain via the protease-cleavable linker. In some embodiments, the protease-cleavable linker comprises a matrix metalloprotease (MMP) cleavage site, a urokinase plasminogen activator cleavage site, a matriptase cleavage site, a legumain cleavage site, a Disintegrin and Metalloprotease (ADAM) cleavage site, or a caspase cleavage site. In some embodiments, the protease-cleavable linker comprises a matrix metalloprotease (MMP) cleavage site. In some embodiments, the MMP cleavage site is selected from an MMP2 cleavage site, an MMP7 cleavage site, an MMP9 cleavage site and an MMP13 cleavage site. In some embodiments, the MMP cleavage site comprises the sequence IPVSLRSG (SEQ ID NO: 19) or GPLGVR (SEQ ID NO: 21).

In some embodiments, the first masking domain comprises the sequence GASTSVDELQAEVDQLEDENYALKTKVAQLRKKVEKLGSIPVSLRSG (SEQ ID NO: 4). In some embodiments, the second masking domain comprises the sequence GASTTVAQLEEKVKTLRAENYELKSEVQRLEEQVAQLGSIPVSLRSG (SEQ ID NO: 3). In some embodiments, the first masking domain comprises the sequence GASTSVDELQAEVDQLEDENYALKTKVAQLRKKVEKLGSIPVSLRSG (SEQ ID NO: 4), and the second masking domain comprises the sequence GASTTVAQLEEKVKTLRAENYELKSEVQRLEEQVAQLGSIPVSLRSG (SEQ ID NO: 3).

In some embodiments, the first masking domain is linked to the amino-terminus of the heavy chain and the second masking domain is linked to the amino-terminus of the light chain. In some embodiments, the first masking domain is linked to the amino-terminus of the light chain and the second masking domain is linked to the amino-terminus of the heavy chain.

In some embodiments, the antibody binds an antigen selected from CD47, CD3, CD19, CD20, CD22, CD30, CD33, CD34, CD40, CD44, CD52, CD70, CD79a, CD123, Her-2, EphA2, lymphocyte associated antigen 1, VEGF or VEGFR, CTLA-4, LIV-1, nectin-4, CD74, SLTRK-6, EGFR, CD73, PD-L1, CD163, CCR4, CD147, EpCam, Trop-2, CD25, C5aR, Ly6D, alpha v integrin, B7H3, B7H4, Her-3, folate receptor alpha, GD-2, CEACAM5, CEACAM6, c-MET, CD266, MUC1, CD10, MSLN, sialyl Tn, Lewis Y, CD63, CD81, CD98, CD166, tissue factor (CD142), CD55, CD59, CD46, CD164, TGF beta receptor 1 (TGFβR1), TGFβR2, TGFβR3, FasL, MerTk, Ax1, Clec12A, CD352, FAP, CXCR3, and CD5.

In some embodiments, the antibody binds CD47. In some embodiments, the antibody comprises a light chain variable region and a heavy chain variable region, wherein the heavy chain variable region comprises HCDR1 comprising SEQ ID NO: 25; HCDR2 comprising SEQ ID NO: 26; and HCDR3 comprising SEQ ID NO: 27; wherein the light chain variable region comprises LCDR1 comprising SEQ ID NO: 31; LCDR2 comprising SEQ ID NO: 32; and LCDR3 comprising SEQ ID NO: 33 or 34. In some embodiments, the heavy chain variable region comprises an amino acid sequence with at least 90%, 91%, 92%, 93%, 94%, 95%, 96%, 97%, 98% or 99% identity to the amino acid sequence of SEQ ID NO: 22.

In some embodiments, the light chain variable region comprises an amino acid sequence with at least 80%, 85%, 90%, 91%, 92%, 93%, 94%, 95%, 96%, 97%, 98% or 99% identity to the amino acid sequence of SEQ ID NO: 23 or 24. In some embodiments, the antibody that binds CD47 comprises HCDR1, HCDR2, HCDR3, LCDR1, LCDR2, and LCDR3 comprising SEQ ID NOs: 25, 26, 27, 31, 32, and 33.

In some embodiments, the antibody that binds CD47 comprises a light chain variable region and a heavy chain variable region, wherein the heavy chain variable region comprises HCDR1 comprising SEQ ID NO: 28; HCDR2 comprising SEQ ID NO: 29; and HCDR3 comprising SEQ ID NO: 30; and wherein the light chain variable region comprises LCDR1 comprising SEQ ID NO: 35; LCDR2 comprising SEQ ID NO: 36; and LCDR3 comprising SEQ ID NO: 37 or 38. In some embodiments, the heavy chain variable region comprises an amino acid sequence with at least 90%, 91%, 92%, 93%, 94%, 95%, 96%, 97%, 98% or 99% identity to the amino acid sequence of SEQ ID NO: 22. In some embodiments, the light chain variable region comprises an amino acid sequence with at least 80%, 85%, 90%, 91%, 92%, 93%, 94%, 95%, 96%, 97%, 98% or 99% identity to the amino acid sequence of SEQ ID NO: 23 or 24. In some embodiments, the antibody comprises HCDR1, HCDR2, HCDR3, LCDR1, LCDR2, and LCDR3 comprising SEQ ID NOs: 28, 29, 30, 35, 36, and 37.

In some embodiments, the heavy chain variable region comprises the amino acid sequence of SEQ ID NO: 22. In some embodiments, the light chain variable region comprises the amino acid sequence of SEQ ID NO: 23 or 24. In some embodiments, the heavy chain variable region comprises the amino acid sequence of SEQ ID NO: 22 and the light chain variable region comprises the amino acid sequence of SEQ ID NO: 23.

In some embodiments, the masked antibody comprises a first masking domain linked to a heavy chain and a second masking domain linked to a light chain, wherein the first masking domain and the heavy chain comprises or consists of the sequence of SEQ ID NO: 39 or SEQ ID NO: 40, and the second masking domain and the light chain comprises or consists of the sequence of SEQ ID NO: 42.

In some embodiments, the antibody that binds CD47 blocks an interaction between CD47 and SIRPα.

In some embodiments, the antibody has reduced core fucosylation. In some embodiments, the antibody is afucosylated.

In some embodiments, the masked antibody is conjugated to a cytotoxic agent. In some embodiments, the cytotoxic agent is an antitubulin agent, a DNA minor groove binding agent, a DNA replication inhibitor, a DNA alkylator, a topoisomerase inhibitor, a NAMPT inhibitor, or a chemotherapy sensitizer. In some embodiments, the cytotoxic agent is an anthracycline, an auristatin, a camptothecin, a duocarmycin, an etoposide, an enediyine antibiotic, a lexitropsin, a taxane, a maytansinoid, a pyrrolobenzodiazepine, a combretastatin, a cryptophysin, or a vinca alkaloid. In some embodiments, the cytotoxic agent is auristatin E, AFP, AEB, AEVB, MMAF, MMAE, paclitaxel, docetaxel, doxorubicin, morpholino-doxorubicin, cyanomorpholino-doxorubicin, melphalan, methotrexate, mitomycin C, a CC-1065 analogue, CBI, calicheamicin, maytansine, an analog of dolastatin 10, rhizoxin, or palytoxin, epothilone A, epothilone B, nocodazole, colchicine, colcimid, estramustine, cemadotin, discodermolide, eleutherobin, a tubulysin, a plocabulin, or maytansine. In some embodiments, the cytotoxic agent is an auristatin. In some embodiments, the cytotoxic agent is MMAE or MMAF.

In some embodiments, the masked antibody exhibits reduced aggregation after at least 1 day, at least 2 days, or at least 3 days at 25° C. compared to the same masked antibody when formulated at pH 7 after the same amount of time at the same temperature.

In some embodiments, less than 2%, less than 1.9%, less than 1.8%, less than 1.7%, less than 1.6%, or less than 1.5% of the antibody in the formulation is demasked. In some embodiments, the amount of demasked antibody in the formulation is determined using Capillary Electrophoresis with Sodium Dodecyl Sulfate (CE-SDS). In some embodiments, CE-SDS is performed under denaturing and reducing conditions. In some embodiments, the amount of demasked light chain is determined based on a CE-SDS electropherogram. In some embodiments, the amount of demasked light chain is determined based on the relative peak area of a peak in a pre-light chain (PreL) region of the electropherogram. In some embodiments, the relative peak area of the peak in the PreL region of the electropherogram is less than 0.8%, or less than 0.7%, or less than 0.6%, or less than 0.5%, or less than 0.4%. In some embodiments, the amount of demasked antibody in the formulation is calculated based on the amount of demasked light chain in the formulation, as measured by CE-SDS.

In some embodiments, a lyophilized formulation comprising a masked antibody is provided, wherein the masked antibody comprises a first masking domain comprising a first coiled-coil domain, wherein the first masking domain is linked to a heavy chain variable region of an antibody and a second masking domain comprising a second coiled-coil domain, wherein the second masking domain is linked to a light chain variable region of the antibody, wherein the first coiled-coil domain comprises the sequence VDELQAEVDQLEDENYALKTKVAQLRKKVEKL (SEQ ID NO: 2), and the second coiled-coil domain comprises the sequence VAQLEEKVKTLRAENYELKSEVQRLEEQVAQL (SEQ ID NO: 1); wherein the formulation comprises a buffer, and wherein upon reconstitution of the lyophilized formulation in water to form an aqueous formulation, the pH of the aqueous formulation is from 3.5 to 4.5.

In some embodiments, the buffer is selected from acetate, succinate, lactate, and glutamate. In some embodiments, upon reconstitution of the lyophilized formulation in water to form an aqueous formulation, the concentration of the buffer in the aqueous formulation is from 10 mM to 100 mM, or from 10 mM to 80 mM, or from 10 mM to 70 mM, or from 10 mM to 60 mM, or from 10 mM to 50 mM, or from 10 mM to 40 mM, or from 20 mM to 100 mM, or from 20 mM to 80 mM, or from 20 mM to 70 mM, or from 20 mM to 60 mM, or from 20 mM to 50 mM, or from 20 mM to 40 mM.

In some embodiments, the formulation comprises at least one cryoprotectant. In some embodiments, at least one cryoprotectant is selected from sucrose, trehalose, mannitol, and glycine. In some embodiments, upon reconstitution of the lyophilized formulation in water to form an aqueous formulation, the total cryoprotectant concentration in the aqueous formulation is 6-12% w/v. In some embodiments, the formulation comprises sucrose or trehalose. In some embodiments, the formulation comprises mannitol and trehalose, or glycine and trehalose.

In some embodiments, the formulation further comprises at least one excipient selected from glycerol, polyethylene glycol (PEG), hydroxypropyl beta-cyclodextrin (HPBCD), polysorbate 20, polysorbate 80, and poloxamer 188 (P188).

In some embodiments, the formulation does not comprise added salt. In some embodiments, does not comprise added NaCl, KCl, or MgCl₂.

In some embodiments, upon reconstitution of the formulation in water to form an aqueous formulation, the concentration of the masked antibody in the aqueous formulation is from 1 to 30 mg/mL, or from 5 to 30 mg/mL, or from 10 to 30 mg/mL, or from 5 to 25 mg/mL, or from 5 to 20 mg/mL, or from 10 to 20 mg/mL, or from 10 to 25 mg/mL, or from 15 to 25 mg/mL.

In some embodiments, upon reconstitution of the formulation in water to form an aqueous formulation, the aqueous formulation comprises 40 mM acetate, 8% sucrose, 0.05% PS80, pH 3.7-4.4. In some embodiments, the formulation comprises 20 mg/mL or 18 mg/mL masked antibody.

In some embodiments, upon reconstitution of the formulation in water to form an aqueous formulation, the aqueous formulation comprises 40 mM glutamate, 8% w/v trehalose dihydrate, and 0.05% polysorbate 80, pH 3.6-4.2. In some embodiments, the formulation comprises 20 mg/mL or 18 mg/mL masked antibody.

In some embodiments, the lyophilized formulation is produced by lyophilizing an aqueous formulation provided herein.

In some embodiments, less than 2%, less than 1.9%, less than 1.8%, less than 1.7%, less than 1.6%, or less than 1.5% of the antibody in the lyophilized formulation is demasked. In some embodiments, the amount of demasked antibody in the lyophilized formulation is determined by reconstituting the formulation in water to form an aqueous formulation, and subjecting the reconstituted aqueous formulation to Capillary Electrophoresis with Sodium Dodecyl Sulfate (CE-SDS). In some embodiments, CE-SDS is performed under denaturing and reducing conditions. In some embodiments, the amount of demasked light chain is determined based on a CE-SDS electropherogram. In some embodiments, the amount of demasked light chain is determined based on the relative peak area of a peak in a pre-light chain (PreL) region of the electropherogram. In some embodiments, the relative peak area of the peak in the PreL region of the electropherogram is less than 0.8%, or less than 0.7%, or less than 0.6%, or less than 0.5%, or less than 0.4%. In some embodiments, the amount of demasked antibody in the lyophilized formulation is calculated based on the amount of demasked light chain in the reconstituted aqueous formulation, as measured by CE-SDS.

In some embodiments, a method for treating cancer, an autoimmune disorder, or an infection in a subject comprises administering to the subject in need thereof a therapeutically effective amount of an aqueous formulation provided herein, or a lyophilized formulation provided herein that has been reconstituted, and optionally diluted, to form a reconstituted aqueous formulation.

In some embodiments, a method for treating a CD47-expressing cancer in a subject comprises administering to the subject a therapeutically effective amount of an aqueous formulation provided herein, or a lyophilized formulation provided herein that has been reconstituted, and optionally diluted, to form a reconstituted aqueous formulation.

In some embodiments, a method for treating a CD47-expressing cancer in a subject comprises:
a) identifying a subject as having a CD47-expressing cancer; and
b) administering to the subject a therapeutically effective amount of an aqueous formulation provided herein or a lyophilized formulation provided herein that has been reconstituted, and optionally diluted, to form a reconstituted aqueous formulation.

In some embodiments, step a) comprises:
i) isolating cancer tissue; and
ii) detecting CD47 in the isolated cancer tissue.

In some embodiments, a method for treating a CD47-expressing cancer in a subject comprises:
a) identifying a subject as having elevated levels of macrophage infiltration in cancer tissue relative to non-cancer tissue; and
b) administering to the subject a therapeutically effective amount of an aqueous formulation provided herein or a lyophilized formulation provided herein that has been reconstituted, and optionally diluted, to form a reconstituted aqueous formulation.

In some embodiments, step a) comprises:
i) isolating cancer tissue and surrounding non-cancer tissue from the subject;
ii) detecting macrophages in the isolated cancer tissue and in non-cancer tissue; and
iii) comparing the amount of staining in the cancer tissue relative to the non-cancer tissue. In some embodiments, the macrophage staining is performed with an anti-CD163 antibody.

In some embodiments, the CD47-expressing cancer is a hematological cancer or a solid cancer. In some embodiments, the CD47-expressing cancer is selected from non-Hodgkin lymphoma, B-lymphoblastic lymphoma; B-cell chronic lymphocytic leukemia/small lymphocytic lymphoma, Richter's syndrome, follicular lymphoma, multiple myeloma, myelofibrosis, polycythemia vera, cutaneous T-cell lymphoma, monoclonal gammopathy of unknown significance (MGUS), myelodysplastic syndrome (MDS), immunoblastic large cell lymphoma, precursor B-lymphoblastic lymphoma, acute myeloid leukemia (AML), and anaplastic large cell lymphoma. In some embodiments, the CD47-expressing cancer is selected from lung cancer, pancreatic cancer, breast cancer, liver cancer, ovarian cancer, testicular cancer, kidney cancer, bladder cancer, spinal cancer, brain cancer, cervical cancer, endometrial cancer, colorectal cancer, anal cancer, esophageal cancer, gallbladder cancer, gastrointestinal cancer, gastric cancer, carcinoma, head and neck cancer, skin cancer, melanoma, prostate cancer, pituitary cancer, stomach cancer, uterine cancer, vaginal cancer and thyroid cancer. In some embodiments, the CD47-expressing cancer is selected from lung cancer, sarcoma, colorectal cancer, head and neck cancer, ovarian cancer, pancreatic cancer, gastric cancer, melanoma, and breast cancer.

In some embodiments, the aqueous formulation provided herein or reconstituted aqueous formulation provided herein is administered in combination with an inhibitor of an immune checkpoint molecule chosen from one or more of programmed cell death protein 1 (PD-1), programmed death-ligand 1 (PD-L1), PD-L2, cytotoxic T lymphocyte-associated protein 4 (CTLA-4), T cell immunoglobulin and mucin domain containing 3 (TIM-3), lymphocyte activation gene 3 (LAG-3), carcinoembryonic antigen related cell adhesion molecule 1 (CEACAM-1), CEACAM-5, V-domain Ig suppressor of T cell activation (VISTA), B and T lymphocyte attenuator (BTLA), T cell immunoreceptor with Ig and ITIM domains (TIGIT), leukocyte-associated immunoglobulin-like receptor 1 (LAIR1), CD160, 2B4 or TGFR. In some embodiments, the aqueous formulation provided herein or reconstituted aqueous formulation provided herein is administered in combination with an agonistic anti-CD40 antibody. In some embodiments, the agonistic anti-CD40 antibody has low fucosylation levels or is afucosylated.

In some embodiments, the aqueous formulation provided herein or reconstituted aqueous formulation provided herein is administered in combination with an antibody drug conjugate (ADC), wherein the antibody of the ADC specifically binds to a protein that is expressed on the extracellular surface of a cancer cell and the antibody is conjugated to a drug-linker comprising a cytotoxic agent. In some embodiments, the cytotoxic agent is an auristatin. In some embodiments, the antibody of the ADC is conjugated to a drug-linker selected from vcMMAE and mcMMAF.

In some embodiments, at least one masking domain comprises a protease-cleavable linker, and wherein the protease-cleavable linker is cleaved in a tumor microenvironment following administration of the aqueous formulation or reconstituted aqueous formulation. In some embodiments, following cleavage in the tumor microenvironment, the released antibody binds its target antigen with an affinity at least about 100-fold stronger than the affinity of the masked antibody for the target antigen. In some embodiments, following cleavage in the tumor microenvironment, the released antibody binds its target antigen with an affinity from 200-fold to 1500-fold stronger than the affinity of the masked antibody for the target antigen.

In some embodiments, the antibody binds CD47, and administration of the aqueous formulation or reconstituted aqueous formulation does not induce hemagglutination in the subj ect.

In some embodiments, a reconstituted aqueous formulation is made by reconstituting the lyophilized formulation provided herein in a clinical diluent. In some embodiments, a reconstituted aqueous formulation is made by reconstituting the lyophilized formulation provided herein in water and then diluting with a clinical diluent. In some embodiments, the clinical diluent is selected from saline, Ringer's solution, lactated Ringer's solution, PLASMA-LYTE 148, and PLASMA-LYTE A.

In some embodiments, a method of making a lyophilized formulation comprising a masked antibody comprises lyophilizing an aqueous formulation provided herein.

In some embodiments, a method of determining the amount of demasked antibody in an aqueous formulation of a masked antibody comprises subjecting a sample of the aqueous formulation to Capillary Electrophoresis with Sodium Dodecyl Sulfate (CE-SDS).

In some embodiments, the masked antibody comprises a first masking domain comprising a first coiled-coil domain, wherein the first masking domain is linked to a heavy chain variable region of an antibody and a second masking domain comprising a second coiled-coil domain, wherein the second masking domain is linked to a light chain variable region of the antibody. In some embodiments, the first coiled-coil domain comprises the sequence VDELQAEVDQLEDENYALKTKVAQLRKKVEKL (SEQ ID NO: 2), and the second coiled-coil domain comprises the sequence VAQLEEKVKTLRAENYELKSEVQRLEEQVAQL (SEQ ID NO: 1).

In some embodiments, the CE-SDS is performed under denaturing and reducing conditions. In some embodiments, the amount of demasked antibody is determined based on a CE-SDS electropherogram. In some embodiments, the amount of demasked antibody is determined based on the amount of demasked light chain. In some embodiments, the amount of demasked light chain is determined based on the relative peak area of a peak in a pre-light chain (PreL) region of the electropherogram.

In some embodiments, the method comprises determining whether the aqueous formulation passes a quality control specification. In some embodiments, the aqueous formulation passes a quality control specification if the amount of demasked light chain determined based on the relative peak area of a peak in a pre-light chain (PreL) region of the electropherogram is less than 0.8%, or less than 0.7%, or less than 0.6%, or less than 0.5%, or less than 0.4%. In some embodiments, the amount of demasked antibody in the aqueous formulation is calculated based on the amount of demasked light chain in the formulation, as measured by CE-SDS. In some embodiments, the aqueous formulation passes a quality control specification if less than 2%, less than 1.9%, less than 1.8%, less than 1.7%, less than 1.6%, or less than 1.5% of the antibody in the aqueous formulation or lyophilized formulation is demasked.

In some embodiments, the aqueous formulation is a reconstituted aqueous formulation. In some embodiments, the reconstituted aqueous formulation is formed by reconstituting a lyophilized formulation in water. In some embodiments, the aqueous formulation is an aqueous formulation or is a reconstituted aqueous formulation formed by reconstituting the lyophilized formulation.

The summary of the disclosure described above is non-limiting, and other features and advantages of the disclosed antibodies and methods of making and using them will be apparent from the following drawings, the detailed description, the examples and the claims.

### BRIEF DESCRIPTION OF THE DRAWINGS

FIGS. 1A-1B show that stability of an anti-CD47 masked antibody (Vel-IPV-hB6H12.3; also called CD47M) is sensitive to pH. (A) Stability of Vel-IPV-hB6H12.3 (measured as percentage high molecular weight [HMW]) after 3 days at 25° C. in formulations of different pH with 150 mM NaCl (salt) or without added salt (no salt). (B) Stability of Vel-IPV-hB6H12.3 over 3 days at 25° C. in formulations at pH 4 and pH 6.

FIG. 2 shows the stability over 24 hours of storage at ambient temperature of Vel-IPV-hB6H12.3 formulated in 20 mM acetate, pH 4, with various excipients. HBCD=hydroxypropyl-beta-cyclodextrin; PS20=polysorbate 20; P188=poloxamer 188; PEG=polyethylene glycol; TMAC=tetramethylammonium chloride; Arg=arginine.

FIG. 3 shows the stability over 2 days at ambient temperature of Vel-IPV-hB6H12.3 at a range of concentrations formulated in 40 mM acetate, pH 4.

FIGS. 4A-4E show stability at 25° C. over 7 days of Vel-IPV-hB6H12.3 in a variety of formulations with 20 mM acetate (A), 20 mM succinate (B), 40 mM lactate (C), or 40 mM glutamate (D, E).

FIGS. 5A-5B show results of design of experiments (DOE) analysis for succinate (A) and acetate (B) formulations. The dotted line shows predicted percentage of BMW Vel-IPV-hB6H12.3 at pH 4.

FIGS. 6A-6C show Vel-IPV-hB6H12.3 stability in 40 mM acetate formulation at different pH. Stability was measured by SE-UPLC (A), charge stability (B), and CE-SDS stability (C). iCIEF=column imaging detection capillary isoelectric focusing; LC+HC=light chain+heavy chain; R CE-SDS=reduced capillary electrophoresis sodium dodecyl sulfate.

FIG. 7 shows stability over time at room temperature of reconstituted drug product (DP) in formulations at various pHs.

FIGS. 8A-8B show stability over time at 5° C. of lyophilized DP as measured by percentage HMW Vel-IPV-hB6H12.3 (A) or percentage acidic variants (B).

FIGS. 9A-9B show stability over time at 5° C. or 25° C. of DP reconstituted in water as measured by percentage HMW Vel-IPV-hB6H12.3 (A) or percentage acidic variants (B).

FIG. 10 show stability at 40° C. of lyophilized DP in formulations with 8% sucrose or 8% trehalose.

FIGS. 11A-11B show stability over time at 40° C. of lyophilized DP as measured by percentage BMW Vel-IPV-hB6H12.3 (A) or percentage acidic variants (B) in various buffers.

FIGS. 12A-12B show stability over 8 hours at room temperature of lyophilized DP reconstituted with water and diluted in saline. (A) Stability of different antibody concentrations. (B) Average stability at 0 hour and after 8 hours incubation in administration devices as measured by percentage HMW Vel-IPV-hB6H12.3 (“HMW”) or potency as measured by percentage relative binding to CD47 (% RB).

FIGS. 13A-13B show data on demasking of Vel-IPV-hB6H12.3. (A) Levels of demasked Vel-IPV-hB6H12.3 increased over a 2-hour demasking reaction with MMP2. (B) The percentage of HMW Vel-IPV-hB6H12.3 over time during the demasking reaction.

FIGS. 14A-14B depict representative cytokine production induced by incubation of cancer patient whole blood samples incubated with hB6H12.3 or Vel-IPV-hB6H12.3 (CD47M) for 20 hours at 37° C. FIG. 13A shows production of IP-10 and FIG. 13B shows production of IL-1RA.

FIG. 15 shows annexin V staining on HT1080 tumor cells from HT1080 xenograft model mice administered hB6H12.3, Vel-IPV-hB6H12.3 (CD47M), or hIgG1 isotype control (“h00 isotype”).

FIG. 16 shows SE-UPLC chromatograms of co-mixed masked and demasked antibody material. The full chromatogram is shown in (A), a zoomed view of the full chromatogram is shown in (B), and a zoomed view of the demasked peak, with an indication of the percentage of demasked antibody material in each sample indicated, is shown in (C).

FIG. 17 shows that the demasked antibody material elutes within the low molecular weight species region of the chromatogram.

FIG. 18 shows a representative CE-SDS electropherogram of a masked antibody sample and a co-mixed sample containing both masked and demasked antibody material. LC indicates masked light chain and HC indicates masked heavy chain.

FIG. 19 shows CE-SDS electropherograms of masked antibody product lots. Full electropherograms are shown in (A) and zoomed view of the PreL region are shown in (B). LC indicates masked light chain and HC indicates masked heavy chain.

FIG. 20 shows CE-SDS electropherograms of masked antibody subjected to 5 stress conditions. Full electropherograms are shown in (A) and zoomed view of the PreL region are shown in (B). LC indicates masked light chain and HC indicates masked heavy chain.

FIG. 21 shows a CE-SDS electropherogram of all co-mixed masked and demasked antibody material (A) and a zoomed view of the demasked light chain (PreL) region and masked light chain region (B).

FIG. 22 shows a linear regression of the percentage of demasked antibody in the co-mixed sample versus the percent time corrected area (TCA) of the demasked light chain (dmLC).

### DETAILED DESCRIPTION

The invention provides formulations comprising antibodies in which variable regions are masked by linkage of the variable region chains to coiled-coil forming polypeptides. The coiled-coil forming polypeptides associate with one another to form coiled coils (i.e., the respective peptides each form coils and these coils are coiled around each other) and, in some embodiments, sterically inhibit binding of the antibody binding site to its target. These coiled-coil polypeptides may be linked to the heavy chain and light chain variable regions of the antibody. Masking of antibodies by this format can reduce binding affinities (and cytotoxic activities in the case of ADC's) by over one hundred-fold, and in some embodiments, can reduce off-target effects. In some instances, however, masked antibodies may aggregate in solution, which may be undesirable in a pharmaceutical formulation. In some embodiments, the present formulations reduce the aggregation of masked antibodies.

Because this coiled-coil masking can be applied to any antibody, as it is independent of the specific CDR and variable region sequences of the antibody and independent of the target or epitope that an antibody binds, the formulations herein are applicable to a wide variety of masked antibodies comprising coiled-coil masking polypeptides.

In some embodiments, the antibody is an anti-CD47 antibody. It may be useful to administer anti-CD47 antibodies to patients in a masked form. For example, anti-CD47 IgG3 antibodies have been known to exhibit toxicities such as peripheral red blood cell depletion and platelet depletion, which decrease their usefulness as effective therapeutics against CD47-associated disorders such as, e.g., CD47 expressing cancers. Masked anti-CD47 antibodies may therefore be less toxic, for example, in that they can be activated by unmasking in the context of a tumor microenvironment, to effectively target the antibodies of the present invention specifically to CD47-expressing solid tumors. Accordingly, the formulations herein are compatible with a variety of anti-CD47 antibodies, such as those specifically disclosed herein.

In certain exemplary embodiments, antibodies are provided that comprise a removable mask (e.g., a mask comprising a coiled coil domain) that blocks binding of the antibody to its antigenic target. In certain embodiments, a coiled-coil domain is attached to the amino-terminus of one or more of the heavy and/or light chains of the antibody via a matrix metalloproteinase (MMP)-cleavable linker sequence. In a tumor microenvironment, for example, altered proteolysis leads to unregulated tumor growth, tissue remodeling, inflammation, tissue invasion, and metastasis (Kessenbrock (2011) Cell 141:52). MMPs represent the most prominent family of proteinases associated with tumorigenesis, and MMPs mediate many of the changes in the microenvironment during tumor progression. Id. Upon exposure of the antibody of the present invention to an MMP, the MMP linker sequence is cleaved, thus allowing removal of the coiled coil mask and enabling the antibody to bind its target antigen in a tumor microenvironment-specific manner.

In other embodiments, such as for use in vitro, such as in medical diagnostics, chemical processing, or industrial uses, masked antibodies may be useful so that antibody activity can be controlled by addition of an exogenous protease to the solution at an appropriate point to cleave off the coiled-coils of the mask and allow the antibodies to bind to their targets. Regardless of the application, however, addition of coiled-coil masks to antibodies could increase the risk of aggregation when the antibodies are stored in concentrated form. Formulations described herein may address this concern by reducing aggregation of solutions comprising the antibodies.

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

Compositions or methods “comprising” one or more recited elements or steps may include other elements or steps not specifically recited. For example, a composition that comprises antibody may contain the antibody alone or in combination with other ingredients.

Compositions or methods “consisting essentially of” one or more steps may include elements or steps not specifically recited so long as any additional element or step does not materially alter the essential nature of the composition or method as recited in the claim. For example, other steps may be included so long as they do not materially alter the overall preparation process, such as wash steps or buffer changes.

Unless otherwise apparent from the context, when a value is expressed as “about” X or “approximately” X, the stated value of X will be understood to be accurate to ±10%.

Solvates in the context of the invention are those forms of the compounds of the invention that form a complex in the solid or liquid state through coordination with solvent molecules. Hydrates are one specific form of solvates, in which the coordination takes place with water. In certain exemplary embodiments, solvates in the context of the present invention are hydrates.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

The term “antibody” denotes immunoglobulin proteins produced by the body in response to the presence of an antigen and that bind to the antigen, as well as antigen-binding fragments and engineered variants thereof. Hence, the term “antibody” includes, for example, intact monoclonal antibodies (e.g., antibodies produced using hybridoma technology) and it also encompasses antigen-binding antibody fragments, such as a F(ab′)₂, a Fv fragment, a diabody, a single-chain antibody, an scFv fragment, or an scFv-Fc. Genetically engineered intact antibodies and fragments such as chimeric antibodies, humanized antibodies, single-chain Fv fragments, single-chain antibodies, diabodies, minibodies, linear antibodies, bispecific or bivalent, multivalent or multi-specific (e.g., bispecific) hybrid antibodies, and the like. Thus, the term “antibody” is used expansively to include any protein that comprises an antigen-binding site of an antibody and is capable of specifically binding to its antigen.

The term “antibody” includes a “naked” antibody that is not bound (i.e., covalently or non-covalently bound) to a masking compound of the invention. The term antibody also embraces a “masked” antibody, which comprises an antibody that is covalently or non-covalently bound to one or more masking compounds such as, e.g., coiled coil peptides, as described further herein. The term antibody includes a “conjugated” antibody or an “antibody-drug conjugate (ADC)” in which an antibody is covalently or non-covalently bound to a pharmaceutical agent, e.g., to a cytostatic or cytotoxic drug. In certain embodiments, an antibody is a naked antibody or antigen-binding fragment that optionally is conjugated to a pharmaceutical agent, e.g., to a cytostatic or cytotoxic drug. In other embodiments, an antibody is a masked antibody or antigen-binding fragment that optionally is conjugated to a pharmaceutical agent, e.g., to a cytostatic or cytotoxic drug.

Antibodies typically comprise a heavy chain variable region and a light chain variable region, each comprising three complementary determining regions (CDRs) with surrounding framework (FR) regions, for a total of six CDRs. An antibody light or heavy chain variable region (also referred to herein as a “light chain variable domain” (“VL domain”) or “heavy chain variable domain” (“VH domain”), respectively) comprises “framework” regions interrupted by three “complementarity determining regions” or “CDRs.” The framework regions serve to align the CDRs for specific binding to an epitope of an antigen. Thus, the term “CDR” refers to the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, FR4.

Naturally occurring antibodies are usually tetrameric and consist of two identical pairs of heavy and light chains. In each pair, the light and heavy chain variable regions (VL and VH) are together primarily responsible for binding to an antigen, and the constant regions are primarily responsible for the antibody effector functions. Five classes of antibodies (IgG, IgA, IgM, IgD, and IgE) have been identified in higher vertebrates. IgG comprises the major class, and it normally exists as the second most abundant protein found in plasma. In humans, IgG consists of four subclasses, designated IgG1, IgG2, IgG3, and IgG4. Each immunoglobulin heavy chain possesses a constant region that comprises constant region protein domains (CH1, hinge, CH2, and CH3; IgG3 also contains a CH4 domain) that are substantially invariant for a given subclass in a species. Antibodies as defined herein, may include these natural forms as well as various antigen-binding fragments, as described above, antibodies with modified heavy chain constant regions, bispecific and multispecific antibodies, and masked antibodies.

The assignment of amino acids to each variable region domain is in accordance with the definitions of Kabat, Sequences of Proteins of Immunological Interest (National Institutes of Health, Bethesda, Md., 1987 and 1991). Kabat also provides a widely used numbering convention (Kabat numbering) in which corresponding residues between different heavy chain variable regions or between different light chain variable regions are assigned the same number. CDRs 1, 2 and 3 of a VL domain are also referred to herein, respectively, as CDR-L1, CDR-L2 and CDR-L3. CDRs 1, 2 and 3 of a VH domain are also referred to herein, respectively, as CDR-H1, CDR-H2 and CDR-H3. If so noted, the assignment of CDRs can be in accordance with IMGT® (Lefranc et al., Developmental & Comparative Immunology 27:55-77; 2003) in lieu of Kabat.

An “antigen-binding site” of an antibody is that portion of an antibody that is sufficient to bind to its antigen. The minimum such region is typically a fragment of a variable domain comprising six CDRs (or three CDRs in the case of a single-domain antibody). In some embodiments, an antigen-binding site of an antibody comprises both a heavy chain variable (VH) domain and a light chain variable (VL) domain that bind to a common epitope. Within the context of the present invention, an antibody may include one or more components in addition to an antigen-binding site, such as, for example, a second antigen-binding site of an antibody (which may bind to the same or a different epitope or to the same or a different antigen), a peptide linker, an immunoglobulin constant region, an immunoglobulin hinge, an amphipathic helix (see Pack and Pluckthun, Biochem. 31: 1579-1584, 1992), a non-peptide linker, an oligonucleotide (see Chaudri et al, FEBS Letters 450:23-26, 1999), a cytostatic or cytotoxic drug, and the like, and may be a monomeric or multimeric protein. Examples of molecules comprising an antigen-binding site of an antibody are known in the art and include, for example, Fv, single-chain Fv (scFv), Fab, Fab′, F(ab′)2, F(ab)c, diabodies, minibodies, nanobodies, Fab-scFv fusions, bispecific (scFv)4-IgG, and bispecific (scFv)2-Fab. (See, e.g., Hu et al, Cancer Res. 56:3055-3061, 1996; Atwell et al., Molecular Immunology 33: 1301-1312, 1996; Carter and Merchant, Curr. Op. Biotechnol. 8:449-454, 1997; Zuo et al., Protein Engineering 13:361-367, 2000; and Lu et al., J. Immunol. Methods 267:213-226, 2002.)

Numbering of the heavy chain constant region is via the EU index as set forth in Kabat (Kabat, Sequences of Proteins of Immunological Interest, National Institutes of Health, Bethesda, Md., 1987 and 1991).

Unless the context dictates otherwise, the term “monoclonal antibody” is not limited to antibodies produced through hybridoma technology. The term “monoclonal antibody” can include an antibody that is derived from a single clone, including any eukaryotic, prokaryotic or phage clone. In particular embodiments, the antibodies described herein are monoclonal antibodies.

The term “chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

The term “humanized VH domain” or “humanized VL domain” refers to an immunoglobulin VH or VL domain comprising some or all CDRs entirely or substantially from a non-human donor immunoglobulin (e.g., a mouse or rat) and variable domain framework sequences entirely or substantially from human immunoglobulin sequences. The non-human immunoglobulin providing the CDRs is called the “donor” and the human immunoglobulin providing the framework is called the “acceptor.” In some instances, humanized antibodies will retain some non-human residues within the human variable domain framework regions to enhance proper binding characteristics (e.g., mutations in the frameworks may be required to preserve binding affinity when an antibody is humanized).

A “humanized antibody” is an antibody comprising one or both of a humanized VH domain and a humanized VL domain. Immunoglobulin constant region(s) need not be present, but if they are, they are entirely or substantially from human immunoglobulin constant regions.

Although humanized antibodies often incorporate all six CDRs (preferably as defined by Kabat or IMGT®) from a mouse antibody, they can also be made with fewer than all six CDRs (e.g., at least 3, 4, or 5) from a mouse antibody (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164: 1432-1441, 2000).

A CDR in a humanized antibody is “substantially from” a corresponding CDR in a non-human antibody when at least 60%, at least 85%, at least 90%, at least 95% or 100% of corresponding residues (as defined by Kabat (or IMGT)) are identical between the respective CDRs. In particular variations of a humanized VH or VL domain in which CDRs are substantially from a non-human immunoglobulin, the CDRs of the humanized VH or VL domain have no more than six (e.g., no more than five, no more than four, no more than three, no more than two, or nor more than one) amino acid substitutions (preferably conservative substitutions) across all three CDRs relative to the corresponding non-human VH or VL CDRs. The variable region framework sequences of an antibody VH or VL domain or, if present, a sequence of an immunoglobulin constant region, are “substantially from” a human VH or VL framework sequence or human constant region, respectively, when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering for the variable region and EU numbering for the constant region), or about 100% of corresponding residues (as defined by Kabat numbering for the variable region and EU numbering for the constant region) are identical. Hence, all parts of a humanized antibody, except the CDRs, are typically entirely or substantially from corresponding parts of natural human immunoglobulin sequences.

Two amino acid sequences have “100% amino acid sequence identity” if the amino acid residues of the two amino acid sequences are the same when aligned for maximal correspondence. Sequence comparisons can be performed using standard software programs such as those included in the LASERGENE bioinformatics computing suite, which is produced by DNASTAR (Madison, Wisconsin). Other methods for comparing two nucleotide or amino acid sequences by determining optimal alignment are well-known to those of skill in the art. (See, e.g., Peruski and Peruski, The Internet and the New Biology: Tools for Genomic and Molecular Research (ASM Press, Inc. 1997); Wu et al. (eds.), “Information Superhighway and Computer Databases of Nucleic Acids and Proteins,” in Methods in Gene Biotechnology 123-151 (CRC Press, Inc. 1997); Bishop (ed.), Guide to Human Genome Computing (2nd ed., Academic Press, Inc. 1998).) Two amino acid sequences are considered to have “substantial sequence identity” if the two sequences have at least about 80%, at least about 85%, at about least 90%, or at least about 95% sequence identity relative to each other.

Percentage sequence identities are determined with antibody sequences maximally aligned by the Kabat numbering convention. After alignment, if a subject antibody region (e.g., the entire variable domain of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Specific binding of an antibody to its target antigen typically refers an affinity of at least about 10⁶, about 10⁷, about 10⁸, about 10⁹, or about 10¹⁰ M⁻¹. Specific binding is detectably higher in magnitude and distinguishable from non-specific binding occurring to at least one non-specific target. Specific binding can be the result of formation of bonds between particular functional groups or particular spatial fit (e.g., lock and key type), whereas nonspecific binding is typically the result of van der Waals forces.

The term “epitope” refers to a site of an antigen to which an antibody binds. An epitope can be formed from contiguous amino acids or noncontiguous amino acids juxtaposed by tertiary folding of one or more proteins. Epitopes formed from contiguous amino acids are typically retained upon exposure to denaturing agents, e.g., solvents, whereas epitopes formed by tertiary folding are typically lost upon treatment with denaturing agents, e.g., solvents. An epitope typically includes at least about 3, and more usually, at least about 5, at least about 6, at least about 7, or about 8-10 amino acids in a unique spatial conformation. Methods of determining spatial conformation of epitopes include, for example, x-ray crystallography and two-dimensional nuclear magnetic resonance. See, e.g., Epitope Mapping Protocols, in Methods in Molecular Biology, Vol. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody bound to its antigen to identify contact residues.

Alternatively, two antibodies have the same epitope if all amino acid mutations in the antigen that reduce or eliminate binding of one antibody reduce or eliminate binding of the other (provided that such mutations do not produce a global alteration in antigen structure). Two antibodies have overlapping epitopes if some amino acid mutations that reduce or eliminate binding of one antibody reduce or eliminate binding of the other antibody.

Competition between antibodies can be determined by an assay in which a test antibody inhibits specific binding of a reference antibody to a common antigen (see, e.g., Junghans et al., Cancer Res. 50: 1495, 1990). A test antibody competes with a reference antibody if an excess of a test antibody inhibits binding of the reference antibody.

Antibodies identified by competition assay (competing antibodies) include antibodies that bind to the same epitope as the reference antibody and antibodies that bind to an adjacent epitope sufficiently proximal to the epitope bound by the reference antibody for steric hindrance to occur. Antibodies identified by a competition assay also include those that indirectly compete with a reference antibody by causing a conformational change in the target protein thereby preventing binding of the reference antibody to a different epitope than that bound by the test antibody.

An antibody effector function refers to a function contributed by an Fc region of an Ig. Such functions can be, for example, antibody-dependent cellular cytotoxicity (ADCC), antibody-dependent cellular phagocytosis (ADCP), or complement-dependent cytotoxicity (CDC). Such function can be affected by, for example, binding of an Fc region to an Fc receptor on an immune cell with phagocytic or lytic activity or by binding of an Fc region to components of the complement system. Typically, the effect(s) mediated by the Fc -binding cells or complement components result in inhibition and/or depletion of the targeted cell. Fc regions of antibodies can recruit Fc receptor (FcR)-expressing cells and juxtapose them with antibody-coated target cells. Cells expressing surface FcR for IgGs including FcγRIII (CD16), FcγRII (CD32) and FcγRIII (CD64) can act as effector cells for the destruction of IgG-coated cells. Such effector cells include monocytes, macrophages, natural killer (NK) cells, neutrophils and eosinophils. Engagement of FcγR by IgG activates ADCC or ADCP. ADCC is mediated by CD16+ effector cells through the secretion of membrane pore-forming proteins and proteases, while phagocytosis is mediated by CD32+ and CD64+ effector cells (see Fundamental Immunology, 4^{th} ed., Paul ed., Lippincott-Raven, N.Y., 1997, Chapters 3, 17 and 30; Uchida et al., J. Exp. Med. 199:1659-69, 2004; Akewanlop et al., Cancer Res. 61:4061-65, 2001; Watanabe et al., Breast Cancer Res. Treat. 53: 199-207, 1999).

In addition to ADCC and ADCP, Fc regions of cell-bound antibodies can also activate the complement classical pathway to elicit CDC. C1q of the complement system binds to the Fc regions of antibodies when they are complexed with antigens. Binding of C1q to cell-bound antibodies can initiate a cascade of events involving the proteolytic activation of C4 and C2 to generate the C3 convertase. Cleavage of C3 to C3b by C3 convertase enables the activation of terminal complement components including C5b, C6, C7, C8 and C9. Collectively, these proteins form membrane-attack complex pores on the antibody-coated cells. These pores disrupt the cell membrane integrity, killing the target cell (see Immunobiology, 6^{th} ed., Janeway et al, Garland Science, N. Y., 2005, Chapter 2).

The term “antibody-dependent cellular cytotoxicity” or “ADCC” refers to a mechanism for inducing cell death that depends on the interaction of antibody-coated target cells with immune cells possessing lytic activity (also referred to as effector cells). Such effector cells include natural killer cells, monocytes/macrophages and neutrophils. The effector cells attach to an Fc region of Ig bound to target cells via their antigen-combining sites. Death of the antibody-coated target cell occurs as a result of effector cell activity.

The term “antibody-dependent cellular phagocytosis” or “ADCP” refers to the process by which antibody-coated cells are internalized, either in whole or in part, by phagocytic immune cells (e.g., by macrophages, neutrophils and/or dendritic cells) that bind to an Fc region of Ig.

The term “complement-dependent cytotoxicity” or “CDC” refers to a mechanism for inducing cell death in which an Fc region of a target-bound antibody activates a series of enzymatic reactions culminating in the formation of holes in the target cell membrane.

Typically, antigen-antibody complexes such as those on antibody-coated target cells bind and activate complement component Cl q, which in turn activates the complement cascade leading to target cell death. Activation of complement may also result in deposition of complement components on the target cell surface that facilitate ADCC by binding complement receptors (e.g., CR3) on leukocytes.

An “antibody-drug conjugate” refers to an antibody conjugated to a cytotoxic agent or cytostatic agent. Typically, antibody-drug conjugates bind to a target antigen on a cell surface, followed by internalization of the antibody-drug conjugate into the cell and subsequent release of the drug into the cell.

Typically, antigen-antibody complexes such as those on antibody-coated target cells bind and activate complement component Cl q, which in turn activates the complement cascade leading to target cell death. Activation of complement may also result in deposition of complement components on the target cell surface that facilitate ADCC by binding complement receptors (e.g., CR3) on leukocytes.

A “cytotoxic effect” refers to the depletion, elimination and/or killing of a target cell. A “cytotoxic agent” refers to a compound that has a cytotoxic effect on a cell, thereby mediating depletion, elimination and/or killing of a target cell. In certain embodiments, a cytotoxic agent is conjugated to an antibody or administered in combination with an antibody. Suitable cytotoxic agents are described further herein.

A “cytostatic effect” refers to the inhibition of cell proliferation. A “cytostatic agent” refers to a compound that has a cytostatic effect on a cell, thereby mediating inhibition of growth and/or expansion of a specific cell type and/or subset of cells. Suitable cytostatic agents are described further herein.

The terms “patient” and “subject” refer to organisms to be treated by the methods described herein and includes human and other mammalian subjects such as non-human primates, mammals (e.g., murines, simians, equines, bovines, porcines, canines, felines, and the like), rabbits, rats, mice, and the like and transgenic species thereof, that receive either prophylactic or therapeutic treatment. In certain exemplary embodiments, a subject is a human patient suffering from or at risk of developing cancer, e.g., a solid tumor, that optionally secretes one or more proteases capable of cleaving a masking domain (e.g., a coiled coil masking domain) of an antibody described herein.

As used herein, the terms, “treat,” “treatment” and “treating” includes any effect, e.g., lessening, reducing, modulating, ameliorating or eliminating, that results in the improvement of the condition, disease, disorder, and the like, or ameliorating a symptom thereof, such as for example, reduced number of cancer cells, reduced tumor size, reduced rate of cancer cell infiltration into peripheral organs, or reduced rate of tumor metastasis or tumor growth.

As used herein, the term “effective amount” refers to the amount of a compound (e.g., an anti-CD47 antibody or masked antibody) sufficient to effect beneficial or desired results. The term “effective amount,” in the context of treatment of a CD47-expressing disorder by administration of an anti-CD47 antibody as described herein, refers to an amount of such antibody that is sufficient to inhibit the occurrence or ameliorate one or more symptoms of a CD47-related disorder (e.g., a CD47-expressing cancer). An effective amount of an antibody is administered in an “effective regimen.” The term “effective regimen” refers to a combination of amount of the antibody being administered and dosage frequency adequate to accomplish prophylactic or therapeutic treatment of the disorder (e.g., prophylactic or therapeutic treatment of a CD47-expressing cancer).

The term “pharmaceutically acceptable” means approved or approvable by a regulatory agency of the Federal or a state government or listed in the U.S. Pharmacopeia or other generally recognized pharmacopeia for use in animals, and more particularly in humans. The term “pharmaceutically compatible ingredient” refers to a pharmaceutically acceptable diluent, adjuvant, excipient, or vehicle with which an antibody is formulated.

The phrase “pharmaceutically acceptable salt,” refers to pharmaceutically acceptable organic or inorganic salts. Exemplary salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene bis-(2 hydroxy-3-naphthoate) salts. A pharmaceutically acceptable salt may further comprise an additional molecule such as, e.g., an acetate ion, a succinate ion or other counterion. A counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

### I. Masking Domains Comprising Coiled-Coils

In certain embodiments, an antibody is associated with a masking domain comprising coiled coil domains (also referred to as a “coiled coil masking domain”) that blocks binding of the antibody to its antigen target. In various embodiments, an antibody associated with a masking domain is referred to as a “masked antibody.”

A coiled coil is a structural motif in proteins and peptides in which two or more alpha-helices wind around each other to form a supercoil. There can be two, three or four helices in a coiled coil bundle and the helices can either run in the same (parallel) or in the opposite (antiparallel) directions.

Coiled coils typically comprise sequence elements of three and four residues whose hydrophobicity pattern and residue composition are compatible with the structure of amphipathic alpha-helices. The alternating three and four residue sequence elements constitute heptad repeats in which the amino acids are designated ‘a,’ ‘b,’ ‘c,’ ‘d,’ ‘e,’ f and ‘g.’ Residues in positions ‘a’ and ‘d’ are generally hydrophobic and form a zig-zag pattern of knobs and holes that interlock with a similar pattern on another strand to form a tight-fitting hydrophobic core. Of the remaining residues, ‘b,’ ‘c’ and ‘f’ tend to be charged. Therefore, the formation of a heptad repeat depends on the physical properties of hydrophobicity and charge that are required at a particular position, not on a specific amino acid. In certain exemplary embodiments, coiled coils of the present invention are formed from two coiled coil-forming peptides.

Examples of consensus formulae for heptad repeats in coiled coil-forming peptides are provided by WO2011034605, incorporated herein by reference in its entirety for all purposes.

Exemplary consensus formulae according to certain embodiments are set forth below:

(X1, X2, X3, X4, X5, X6, X7)n, wherein: Formula 1:
X1 is a hydrophobic amino acid or asparagine;
X2, X3 and X6 are any amino acid;
X4 is a hydrophobic amino acid;
X5 and X7 are each a charged amino acid residue; and
n is a positive integer.

(X1′, X2′, X3′, X4′, X5, X6, X7)n, wherein: Formula 2:
X1′ is a hydrophobic amino acid or asparagine;
X2′, X′3 and X′6 are each any amino acid residue;
X4′ is hydrophobic amino acid;
X5′ and X7′ are each a charged amino acid residue;
wherein n in formula 1 and 2 is greater or equal to 2; and
n is a positive integer.

In certain embodiments in which peptides of Formula 1 and Formula 2 form a coiled coil, X5 of Formula 1 is opposite in charge to X′7 of Formula 2, and X7 or Formula 1 is opposite in charge to X′5 of Formula 2. Heptad repeats within a coiled coil forming peptide can be the same or different from each other while conforming to Formula 1 and/or 2.

Coiled coils can be homodimeric or heterodimeric. Examples of peptides that can form coiled coil according to certain exemplary embodiments are shown in Table 1 below (SEQ ID NOs: 1-4). The peptide sequences can be used as is, or their components can be used in other combinations. For example, the Vel coiled coil-forming peptide can be used with other linker sequences. Sequences shown for light chains can also be used with heavy chains and vice versa.

In certain exemplary embodiments, a bivalent antibody comprising two light and heavy chain pairs is provided, wherein the amino-termini of one or more of the light chains and/or the heavy chains are linked via linkers comprising a protease cleavage site to coiled coil-forming peptides that associate to form a coiled coil, reducing binding affinity of the light and heavy chain pair to a target. Optionally, the peptides associate without forming a disulfide bridge.

Optionally, the two light and heavy chain pairs are the same. Optionally, the two light and heavy chain pairs are different. Optionally, the light chains include a light chain variable region and light chain constant region and the heavy chains include a heavy chain variable region and heavy chain constant region. Optionally, the heavy chain region includes CH1, hinge, CH2 and CH3 regions. Optionally, the two light chain are linked to a first heterologous peptide and the two heavy chains to a second heterologous peptide.

Optionally, the protease cleavage site is an MMP1, MMP2, and/or MMP12 cleavage site.

In some cases, antigen binding is reduced at least 100-fold by the presence of a masking domain (e.g., a coiled coil masking domain). In some embodiments, antigen binding is reduced 200-1500-fold by the presence of a masking domain (e.g., a coiled coil masking domain). In some embodiments, cytotoxicity of the conjugate is reduced at least 100-fold by the presence of a masking domain (e.g., a coiled coil masking domain). In some embodiments, cytotoxicity of the conjugate is reduced at least 200-1500-fold by the presence of a masking domain (e.g., a coiled coil masking domain).

Optionally, the coiled coil forming peptides are linked to the amino-termini of the heavy and light chains in the same orientation. Optionally, the coiled coil-forming peptides are linked to the amino-termini of the heavy and light chains in opposing orientations. Optionally, multiple copies of the coiled coil forming peptide are linked in tandem to the amino-termini of the heavy and light chains.

In some embodiments, a masking domain comprises a VelA coiled-coil domain (SEQ ID NO: 1). In some embodiments, a masking domain comprises a VelB coiled-coil domain (SEQ ID NO: 2). In some embodiments, a masked antibody comprises a first masking domain comprising a VelA coiled-coil domain and a second masking domain comprising a VelB coiled-coil domain, wherein the first masking domain is linked to the light chain and the second masking domain is linked to the heavy chain, or vice versa. In some embodiments, each masking domain is linked to the amino-terminus of the heavy chain or light chain.

TABLE 1
Nonlimiting exemplary coiled-coil masking domains

| Description | Sequence | SEQ ID NO |
|---|---|---|
| VelA coiled-coil | VAQLEEKVKTLRAENYELKSEVQRLEEQVAQL | 1 |
| VelB coiled-coil | VDELQAEVDQLEDENYALKTKVAQLRKKVEKL | 2 |
| VelA-IPV | GASTTVAQLEEKVKTLRAENYELKSEVQRLEEQVAQLGSIPVSLRSG | 3 |
| VelB-IPV | GASTSVDELQAEVDQLEDENYALKTKVAQLRKKVEKLGSIPVSLRSG | 4 |

In certain exemplary embodiments, amino acid substitutions in a variant peptide that forms a coiled coil are conservative substitutions. For purposes of classifying amino acids substitutions as conservative or nonconservative, the following amino acid substitutions are considered conservative substitutions: serine substituted by threonine, alanine, or asparagine; threonine substituted by proline or serine; asparagine substituted by aspartic acid, histidine, or serine; aspartic acid substituted by glutamic acid or asparagine; glutamic acid substituted by glutamine, lysine, or aspartic acid; glutamine substituted by arginine, lysine, or glutamic acid; histidine substituted by tyrosine or asparagine; arginine substituted by lysine or glutamine; methionine substituted by isoleucine, leucine or valine; isoleucine substituted by leucine, valine, or methionine; leucine substituted by valine, isoleucine, or methionine; phenylalanine substituted by tyrosine or tryptophan; tyrosine substituted by tryptophan, histidine, or phenylalanine; proline substituted by threonine; alanine substituted by serine; lysine substituted by glutamic acid, glutamine, or arginine; valine substituted by methionine, isoleucine, or leucine; and tryptophan substituted by phenylalanine or tyrosine. Conservative substitutions can also mean substitutions between amino acids in the same class. Classes are as follows: Group I (hydrophobic side chains): met, ala, val, leu, ile; Group II (neutral hydrophilic side chains): cys, ser, thr; Group III (acidic side chains): asp, glu; Group IV (basic side chains): asn, gin, his, lys, arg; Group V (residues influencing chain orientation): gly, pro; and Group VI (aromatic side chains): trp, tyr, phe.

### Linkers and Cleavage Sites

In certain embodiments of the invention, a masking domain comprises a linker, which is located between the coiled-coil domain and the antibody chain to which the coiled-coil domain is attached. The linkers can be any segments of amino acids conventionally used as linker for joining peptide domains. Suitable linkers can vary in length, such as from 1-20, 2-15, 3-12, 4-10, 5, 6, 7, 8, 9 or 10. Some such linkers include a segment of polyglycine. Some such linkers include one or more serine residues, often at positions flanking the glycine residues. Other linkers include one or more alanine residues. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11173-142 (1992)). Some exemplary linkers are in the form S(G)nS, wherein n is from 5-20. Other exemplary linkers are (G)n, glycine-serine polymers (including, for example, (GS)n, (GSGGS)n [(GSGGS) is SEQ ID NO: 5) and (GGGS)n, [(GGGS) is SEQ ID NO: 6) where n is an integer of at least one), glycine-alanine polymers, alanine-serine polymers, and other flexible linkers known in the art. Some examples of linkers are Ser-(Gly)10-Ser (SEQ ID NO: 7), Gly-Gly-Ala-Ala (SEQ ID NO: 8), Gly-Gly-Gly-Gly-Ser (SEQ ID NO: 9), Leu-Ala-Ala-Ala-Ala (SEQ ID NO: 10), Gly-Gly-Ser-Gly (SEQ ID NO: 11), Gly-Gly-Ser-Gly-Gly (SEQ ID NO: 12), Gly-Ser-Gly-Ser-Gly (SEQ ID NO: 13), Gly-Ser-Gly-Gly-Gly (SEQ ID NO: 14), Gly-Gly-Gly-Ser-Gly (SEQ ID NO: 15), Gly-Ser-Ser-Ser-Gly (SEQ ID NO: 16), and the like.

The protease site is preferably recognized and cleaved by a protease expressed extracellularly so it contacts a masked antibody, releasing the masked antibody and allowing it to contact its target, such as a receptor extracellular domain or soluble ligand. Several matrix metalloproteinase sites (MMP1-28) are suitable. MMPs play a role in tissue remodeling and are implicated in neoplastic processes such as morphogenesis, angiogenesis and metastasis. Some exemplary protease sites are PLG-XXX (SEQ ID NO: 17), a well-known endogenous sequence for MMPs, PLG-VR (SEQ ID NO: 18) (WO2014193973) and IPVSLRSG (SEQ ID NO: 19) (Turk et al., Nat. Biotechnol., 2001, 19, 661-667), LSGRSDNY (SEQ ID NO: 20) (Cytomyx) and GPLGVR (SEQ ID NO: 21) (Chang et al., Clin. Cancer Res. 2012 Jan 1; 18(1):238-47). Additional examples of MMPs are provided in US 2013/0309230, WO 2009/025846, WO 2010/081173, WO 2014/107599, WO 2015/048329, US 20160160263, and Ratnikov et al., Proc. Natl. Acad. Sci. USA, 111: E4148-E4155 (2014).

TABLE 2
Protease cleavage sequences. The MMP-cleavage
site is indicated by * while the uPA/matriptase/
legumain cleavage sites are indicated by **.

| Cleavage Site Name | Sequence |
|---|---|
| M2 | GPLG*VR** (SEQ ID NO: 21) |
| IPV | IPVS*LR**SG (SEQ ID NO: 19) |

In some embodiments, a masking domain comprises a coiled-coil domain, a linker, and a protease cleavage sequence. In some such embodiments, a masking domain is VelA-IPV (SEQ ID NO: 3), wherein the coiled-coil domain is VelA (SEQ ID NO: 1), the linker is GS, and the protease cleavage sequence is IPVSLRSG (SEQ ID NO: 19). In some embodiments, a masking domain comprises a coiled-coil domain, a linker, and a protease cleavage sequence. In some such embodiments, a masking domain is VelB-IPV (SEQ ID NO: 4), wherein the coiled-coil domain is VelB (SEQ ID NO: 2), the linker is GS, and the protease cleavage sequence is IPVSLRSG (SEQ ID NO: 19).

In some embodiments, a first masking domain is a VelA-IPV masking domain (SEQ ID No: 3), which includes an MMP protease site, and a second masking domain is a VelB-IPV masking domain (SEQ ID NO: 4), which also includes an MMP protease site. In some embodiments, the first masking domain is linked to the light chain and the second masking domain is linked to a heavy chain, or vice versa. In some embodiments, each masking domain is linked to the amino-terminus of the heavy chain or light chain.

### II. Pharmaceutical Compositions and Formulations

For therapeutic use, a masked antibody is preferably combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Accordingly, masked antibody formulations of the present invention can comprise at least one of any suitable excipients, such as, but not limited to, diluent, binder, stabilizer, buffers, salts, lipophilic solvents, preservative, adjuvant or the like. Pharmaceutically acceptable excipients are preferred. Non-limiting examples of, and methods of preparing such sterile solutions are well known in the art, such as, but not limited to, those described in Gennaro, Ed., Remington's Pharmaceutical Sciences, 18th Edition, Mack Publishing Co. (Easton, Pa.) 1990. Pharmaceutically acceptable carriers can be routinely selected that are suitable for the mode of administration, solubility and/or stability of the antibody molecule, fragment or variant composition as well known in the art or as described herein.

In some embodiments, formulations of masked antibodies are aqueous formulations. In other embodiments, the formulations are lyophilized. In either case, the formulations may comprise a buffer as well as masked antibodies comprising a first and a second masking domain, these domains being linked to the heavy chain variable region and to the light chain variable region of the antibody, respectively. In some embodiments, the masking domains comprise coiled-coil forming polypeptides. Accordingly, in some embodiments, the masking antibodies comprise a first masking domain comprising a coiled-coil domain, which is linked to a heavy chain variable region of the antibody and a second masking domain comprising a coiled-coil domain, which is linked to a light chain variable region of the antibody, wherein the first coiled-coil domain comprises the sequence VDELQAEVDQLEDENYALKTKVAQLRKKVEKL (SEQ ID NO: 2), and the second coiled-coil domain comprises the sequence VAQLEEKVKTLRAENYELKSEVQRLEEQVAQL (SEQ ID NO: 1). In some embodiments, the first masking domain comprises the sequence GASTSVDELQAEVDQLEDENYALKTKVAQLRKKVEKLGSIPVSLRSG (SEQ ID NO: 4) and/or the second masking domain comprises the sequence GASTTVAQLEEKVKTLRAENYELKSEVQRLEEQVAQLGSIPVSLRSG (SEQ ID NO: 3).

In some embodiments, the pH of the formulation is from 3.5 to 4.3. In some embodiments, the buffer is selected from acetate, succinate, lactate, and glutamate, or a mixture of two or more of these ions, and its concentration may optionally be, for example 15-50 mM, such as 15-30 mM, 15-25 mM, 20-50 mM, 20-40 mM, 30-50 mM, 20-30 mM, or 30-40 mM. In some embodiments, the buffer consists essentially of acetate, succinate, lactate, and glutamate, or a mixture of two or more of these ions. In some embodiments, the formulation also comprises a cryoprotectant, which may, for example, include a sugar, sugar alcohol, or amino acid, or a mixture thereof. In some embodiments, the cryoprotectant may include sucrose, trehalose, mannitol, or glycine, or a mixture of two or more of those substances. In some embodiments, the cryoprotectant consists essentially of sucrose, trehalose, mannitol, or glycine, or a mixture of two or more of those substances. In some embodiments, the cryoprotectant concentration is 6-12% w/v, such as 6-10%, 8-12%, 6-8%, 8-10%, or 10-12%.

In some embodiments, the formulation may further comprise a surfactant, such as one or more of glycerol, polyethylene glycol (PEG), hydroxypropyl beta-cyclodextrin (HPBCD), polysorbate 20, polysorbate 80, or poloxamer 188 (P188).

In some embodiments, a formulation provided herein comprises less than 100 mM, or less than 90 mM, or less than 80 mM, or less than 70 mM, or less than 60 mM, or less than 50 mM, less than 40 mM, less than 30 mM, less than 20 mM, or less than 10 mM salt. In some embodiments, the concentration of NaCl in a formulation provided herein is less than 100 mM, or less than 90 mM, or less than 80 mM, or less than 70 mM, or less than 60 mM, or less than 50 mM, less than 40 mM, less than 30 mM, less than 20 mM, or less than 10 mM. In some embodiments, the concentration of KCl in a formulation provided herein is less than 100 mM, or less than 90 mM, or less than 80 mM, or less than 70 mM, or less than 60 mM, or less than 50 mM, less than 40 mM, less than 30 mM, less than 20 mM, or less than 10 mM. In some embodiments, the concentration of MgCl₂ in a formulation provided herein is less than 100 mM, or less than 90 mM, or less than 80 mM, or less than 70 mM, or less than 60 mM, or less than 50 mM, less than 40 mM, less than 30 mM, less than 20 mM, or less than 10 mM.

In various embodiments, the formulation does not comprise added salt.

In some embodiments, the concentration of the antibody in an aqueous formulation herein, or in a reconstitution of a lyophilized formulation as described herein is from 1 to 30 mg/mL, from 5 to 30 mg/mL, or from 10-30 mg/mL.

Formulations may also contain at least one known preservative, optionally selected from at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3, 0.4, 0.5, 0.9, or 1.0%), 0.1-3% benzyl alcohol (e.g., 0.5, 0.9, 1.1., 1.5, 1.9, 2.0, or 2.5%), 0.001-0.5% thimerosal (e.g., 0.005 or 0.01%), 0.001-2.0% phenol (e.g., 0.05, 0.25, 0.28, 0.5, 0.9, or 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, or 1.0%), and the like.

A nonlimiting exemplary formulation comprises 40 mM acetate, 8% sucrose, 0.05% PS80, pH 3.7-4.4, and 10-30 mg/mL, or about 20 mg/mL, or about 18 mg/mL of a masked antibody. In some embodiments, the masked antibody is Vel-IPV-hB6H12.3, comprising a heavy chain comprising the amino acid sequence of SEQ ID NO: 39 or 40, and a light chain comprising the amino acid sequence of SEQ ID NO: 42.

A further nonlimiting exemplary formulation comprises 40 mM glutamate, 8% w/v trehalose dihydrate, and 0.05% polysorbate 80, pH 3.6-4.2, and 10-30 mg/mL, or about 20 mg/mL, or about 18 mg/mL of a masked antibody. In some embodiments, the masked antibody is Vel-IPV-hB6H12.3, comprising a heavy chain comprising the amino acid sequence of SEQ ID NO: 39 or 40, and a light chain comprising the amino acid sequence of SEQ ID NO: 42.

Pharmaceutical formulations of a masked antibody as disclosed herein can be presented in a dosage unit form, or can be stored in a form suitable for supplying more than one unit dose. A pharmaceutical composition should be formulated to be compatible with its intended route of administration. Lyophilized formulations are typically reconstituted in solution prior to administration or use, whereas aqueous formulations may be “ready to use,” meaning that they are administered directly, without being first diluted for example, or can be diluted in saline or another solution prior to use.

Examples of routes of administration are intravenous (IV), intradermal, intratumoral, inhalation, transdermal, topical, transmucosal, and rectal administration. The phrases “parenteral administration” and “administered parenterally” as used herein means modes of administration other than enteral and topical administration, usually by injection, and includes, without limitation, intravenous, intramuscular, subcutaneous, intraarterial, intrathecal, intracapsular, intraorbital, intravitreous, intracardiac, intradermal, intraperitoneal, transtracheal, inhaled, subcutaneous, subcuticular, intraarticular, subcapsular, subarachnoid, intraspinal, epidural and intrasternal injection and infusion.

Pharmaceutical formulations are preferably sterile. Sterilization can be accomplished by any suitable method, e.g., filtration through sterile filtration membranes. Where the composition is lyophilized, filter sterilization can be conducted prior to or following lyophilization and reconstitution.

In some embodiments, an aqueous formulation comprising the masked antibody exhibits reduced aggregation after at least 1 day at 25° C. compared to the same formulation at pH 7 at the same temperature after the same time period. In some embodiments, an aqueous formulation comprising the masked antibody exhibits reduced aggregation after at least 2 days at 25° C. compared to the same masked antibody formulated at pH 7 at the same temperature after the same time period. In some embodiments, an aqueous formulation comprising the masked antibody exhibits reduced aggregation after at least 3 days at 25° C. compared to the same masked antibody formulated at pH 7 at the same temperature after the same time period. In some embodiments, an aqueous reconstitution of a lyophilized formulation comprising the masked antibody exhibits reduced aggregation after at least 1 day at 25° C. compared to the same masked antibody formulated at pH 7 at the same temperature after the same time period. In some embodiments, an aqueous reconstitution of a lyophilized formulation comprising the masked antibody exhibits reduced aggregation after at least 2 days at 25° C. compared to the same masked antibody formulated at pH 7 at the same temperature after the same time period. In some embodiments, an aqueous reconstitution of a lyophilized formulation comprising the masked antibody exhibits reduced aggregation after at least 3 days at 25° C. compared to the masked antibody formulated formulation at pH 7 at the same temperature after the same time period.

The present invention also provides a kit, comprising packaging material and at least one vial comprising an aqueous formulation of masked antibody as described herein. The kit may further comprise instructions for use and/or a diluent solution if the antibody formulation must be diluted prior to use. The present invention also provides a kit, comprising packaging material and at least one vial comprising a lyophilized formulation of masked antibody as described herein. The kit may further comprise instructions for use, a reconstitution solution for reconstituting the antibody into solution, and/or a diluent solution if the antibody formulation must be further diluted after reconstitution.

### III. Exemplary Antibodies

Antibodies include non-human, humanized, human, chimeric, and veneered antibodies, nanobodies, dAbs, scFV's, Fabs, and the like. Some such antibodies are immunospecific for a cancer cell antigen, preferably one on the cell surface internalizable within a cell on antibody binding. In some embodiments, the antibody portion of a masked antibody binds a therapeutic antigen. Such therapeutic antigens include antigens that may be targeted for treatment of any disease or disorder, including, but not limited to, cancer, autoimmune disorders, and infections.

Targets to which antibodies can be directed include receptors on cancer cells and their ligands or counter-receptors (i.e., tumor-associated antigens). Such targets include, but are not limited to, CD3, CD19, CD20, CD22, CD30, CD33, CD34, CD40, CD44, CD47, CD52, CD70, CD79a, CD123, Her-2, EphA2, lymphocyte associated antigen 1, VEGF or VEGFR, CTLA-4, LIV-1, nectin-4, CD74, SLTRK-6, EGFR, CD73, PD-L1, CD163, CCR4, CD147, EpCam, Trop-2, CD25, C5aR, Ly6D, alpha v integrin, B7H3, B7H4, Her-3, folate receptor alpha, GD-2, CEACAMS, CEACAM6, c-MET, CD266, MUC1, CD10, MSLN, sialyl Tn, Lewis Y, CD63, CD81, CD98, CD166, tissue factor (CD142), CD55, CD59, CD46, CD164, TGF beta receptor 1 (TGFβR1), TGFβR2, TGFβR3, FasL, MerTk, Ax1, Clec12A, CD352, FAP, CXCR3, and CD5.

In some embodiments, a masked antibody provided herein may be useful for treating an autoimmune disease. Nonlimiting antigens that may be bound by an antibody useful for treating an autoimmune disease include TNF-α, IL-1, IL-2R, IL-6, IL-12, IL-23, IL-17, IL-17R, BLyS, CD20, CD52, α4β7 integrin, and α4-integrin.

Some examples of commercial antibodies and their targets suitable for use in the masked antibodies described herein include, but are not limited to, brentuximab or brentuximab vedotin, CD30, alemtuzumab, CD52, rituximab, CD20, trastuzumab Her/neu, nimotuzumab, cetuximab, EGFR, bevacizumab, VEGF, palivizumab, RSV, abciximab, GpIIb/IIIa, infliximab, adalimumab, certolizumab, golimumab TNF-alpha, baciliximab, daclizumab, IL-2R, omalizumab, IgE, gemtuzumab or vadastuximab, CD33, natalizumab, VLA-4, vedolizumab alpha4beta7, belimumab, BAFF, otelixizumab, teplizumab CD3, ofatumumab, ocrelizumab CD20, epratuzumab CD22, alemtuzumumab CD52, eculizumab C5, canakimumab IL-1beta, mepolizumab IL-5, reslizumab, tocilizumab IL-6R, ustekinumab, briakinumab IL-12, 23, hBU12 (CD19) (US20120294853), humanized 1F6 or 2F12 (CD70) (US20120294863), BR2-14a and BR2-22a (LIV-1) (WO2012078688).

### Exemplary Anti-CD47 Antibodies

The present formulations may comprise masked versions of isolated, recombinant and/or synthetic anti-CD47 human, primate, rodent, mammalian, chimeric, humanized and/or CDR-grafted antibodies. In certain exemplary embodiments, the formulations herein comprise masked humanized anti-CD47 IgG1 antibodies.

In particular embodiments of the invention, the humanized anti-CD47 antibodies have one or more of the following activities: 1) enhanced antigen binding relative to a reference antibody (e.g., a murine parental antibody); 2) enhanced Antibody Dependent Cellular Cytotoxicity (ADCC) relative to a reference antibody (e.g., a murine parental antibody); 3) enhanced phagocytosis (e.g., Antibody Dependent Cellular Phagocytosis (ADCP)) relative to a reference antibody (e.g., a murine parental antibody); 4) reduced red blood cell hemagglutination (HA), relative to a reference antibody (e.g., a murine parental antibody); 5) binding to a three-dimensional (i.e., non-linear) CD47 epitope. Antibodies hB6H12.3 and hB6H12.3 (deamidation mutant) have one or more, or all, of the foregoing properties, wherein the reference antibody is mB6H12. In some embodiments, antibody hB6H12.3 has at least the property of resulting in reduced red blood cell HA relative to murine B6H12 antibody.

Exemplary anti-CD47 antibodies that may be included in the masked antibodies herein include the CD47 antibody heavy chain/light chain pair of hB6H12.3 (hvH1/hvK3) or hB6H12.3 (deamidation mutant) (hvH1/hvK3 G91A). Exemplary anti-CD47 antibody heavy chain variable region sequences, light chain variable regions, heavy chain CDRs and light chain CDRs can be found at Table 3-Table 8. The amino acid sequences for the heavy chain and light chain of an exemplary humanized anti-CD47 antibody can be found at Table 9.

TABLE 3
Heavy chain variable sequence of hB6H12.3 and hB6H12.3 (deamidation
mutant). Kabat CDRs are underlined, and IMGT CDRs are bolded.

| Heavy Chain | Sequence |
|---|---|
| hvH1 | EVQLLESGGGLVQPGGSLRLSCAASGFTFSGYGMSWVRQAPGKRLEW |
| | VATITSGGTYTYYPDSVKGRFTISRDNSKNTLYLQMNSLRAEDTAIYFC |
| | ARSLAGNAMDYWGQGTLVTVSS (SEQ ID NO: 22) |

TABLE 4
Light chain variable sequence of hB6H12.3 and hB6H12.3 (deamidation mutant).
Kabat CDRs are underlined, and IMGT CDRs are bolded.

| Light Chain | Sequence |
|---|---|
| hvK3 | EIVMTQSPDFQSVTPKEKVTLTCRASQTISDYLHWYQQKPDQSPKLLIK |
| | FASQSISGVPSRFSGSGSGSDFTLTINSLEAEDAATYYCQNGHGFPRTFG |
| | QGTKLEIK(R) (SEQ ID NO: 23) |
| hvK3 (G91A) | EIVMTQSPDFQSVTPKEKVTLTCRASQTISDYLHWYQQKPDQSPKLLIK |
| | FASQSISGVPSRFSGSGSGSDFTLTINSLEAEDAATYYC [symbol] FG |
| | QGTKLEIKR (SEQ ID NO: 24) |

TABLE 5
Heavy chain CDR sequences of hB6H12.3 and hB6H12.3
(deamidation mutant) (Kabat).

| CDR | Sequence |
|---|---|
| hvH1 HCDR1 (Kabat) | GYGMS (SEQ ID NO: 25) |
| hvH1 HCDR2 (Kabat) | TITSGGTYTYYPDSVKG |
| | (SEQ ID NO: 26) |
| hvH1 HCDR3 (Kabat) | SLAGNAMDY (SEQ ID NO: 27) |

TABLE 6
Heavy chain CDR sequences of hB6H12.3 and hB6H12.3
(deamidation mutant)(IMGT).

| CDR | Sequence |
|---|---|
| hvH1 HCDR1 (IMGT) | GFTFSGYG (SEQ ID NO: 28) |
| hvH1 HCDR2 (IMGT) | ITSGGTYT (SEQ ID NO: 29) |
| hvH1 HCDR3 (IMGT) | ARSLAGNAMDY (SEQ ID NO: 30) |

TABLE 7
Light chain CDR sequences of hB6H12.3
and hB6H12.3 (deamidation mutant)
(Kabat).

| CDR | Sequence |
|---|---|
| hvK3 LCDR1 (Kabat) | RASQTISDYLH |
| | (SEQ ID NO: 31) |
| hvK3 LCDR2 (Kabat) | FASQSIS |
| | (SEQ ID NO: 32) |
| hvK3 LCDR3 (Kabat) | QNGHGFPRT |
| | (SEQ ID NO: 33) |
| hvK3 (G91A) LCDR3 | QNAHGFPRT |
| (Kabat) | (SEQ ID NO: 34) |

TABLE 8
Light chain CDR sequences of hB6H12.3 and hB6H12.3
(deamidation mutant) (IMGT).

| CDR | Sequence |
|---|---|
| hvK3 LCDR1 (IMGT) | QTISDY (SEQ ID NO: 35) |
| hvK3 LCDR2 (IMGT) | FAS (SEQ ID NO: 36) |
| hvK3 LCDR3 (IMGT) | QNGHGFPRT (SEQ ID NO: 37) |
| hvK3 (G91A) LCDR3 (IMGT) | QNAHGFPRT (SEQ ID NO: 38) |

TABLE 9
Complete heavy and light chain sequences of a masked anti-CD47 antibody
according to a preferred embodiment of the invention. Heavy chain and light
chain sequences are in plain text, masking sequences are in bold text, and
protease cleavage sequences are underlined.

| Antibody | |
|---|---|
| Chain | Sequence |
| Heavy | QGASTSVDELQAEVDQLEDENYALKTKVAQLRKKVEKLGSIPVSLRSGE |
| Chain | VQLLESGGGLVQPGGSLRLSCAASGFTFSGYGMSWVRQAPGKRLEWVATIT |
| version 1 | SGGTYTYYPDSVKGRFTISRDNSKNTLYLQMNSLRAEDTAIYFCARSLAGN |
| | AMDYWGQGTLVTVSSASTKGPSVFPLAPSSKSTSGGTAALGCLVKDYFPEP |
| | VTVSWNSGALTSGVHTFPAVLQSSGLYSLSSVVTVPSSSLGTQTYICNVNHK |
| | PSNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSRDELT |
| | KNQVSLTCLVKGFYPSDIAVEWESNGQPENNYKTTPPVLDSDGSFFLYSKLT |
| | VDKSRWQQGNVFSCSVMHEALHNHYTQK (SEQ ID NO: 39) |
| Heavy | QGASTSVDELQAEVDQLEDENYALKTKVAQLRKKVEKLGSIPVSLRSGE |
| Chain | VQLLESGGGLVQPGGSLRLSCAASGFTFSGYGMSWVRQAPGKRLEWVATIT |
| version 2 | SGGTYTYYPDSVKGRFTISRDNSKNTLYLQMNSLRAEDTAIYFCARSLAGN |
| | AMDYWGQGTLVTVSSASTKGPSVFPLAPSSKSTSGGTAALGCLVKDYFPEP |
| | VTVSWNSGALTSGVHTFPAVLQSSGLYSLSSVVTVPSSSLGTQTYICNVNHK |
| | PSNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPPSRDELT |
| | KNQVSLTCLVKGFYPSDIAVEWESNGQPENNYKTTPPVLDSDGSFFLYSKLT |
| | VDKSRWQQGNVFSCSVMHEALHNHYTQ (SEQ ID NO: 40) |
| Heavy | QGASTSVDELQAEVDQLEDENYALKTKVAQLRKKVEKLGS (SEQ ID NO: |
| Chain | 41) |
| masking | |
| sequence | |
| Light | QGASTTVAQLEEKVKTLRAENYELKSEVQRLEEQVAQLGSIPVSLRSGE |
| Chain | IVMTQSPDFQSVTPKEKVTLTCRASQTISDYLHWYQQKPDQSPKLLIKFASQ |
| | SISGVPSRFSGSGSGSDFTLTINSLEAEDAATYYCQNGHGFPRTFGQGTKLEI |
| | KRTVAAPSVFIFPPSDEQLKSGTASVVCLLNNFYPREAKVQWKVDNALQSG |
| | NSQESVTEQDSKDSTYSLSSTLTLSKADYEKHKVYACEVTHQGLSSPVTKSF |
| | NRGEC (SEQ ID NO: 42) |
| Light | QGASTTVAQLEEKVKTLRAENYELKSEVQRLEEQVAQLGS (SEQ ID NO: |
| Chain | 43) |
| masking | |
| sequence | |

### hB6H12.3

In certain exemplary embodiments, an anti-CD47 antibody comprises CDRs from a HCVR set forth as SEQ ID NO: 22 and/or CDRs from a LCVR set forth as SEQ ID NO: 23. In other embodiments, an anti-CD47 antibody comprises heavy chain CDRs of SEQ ID NOs: 25, 26 and 27 and/or light chain CDRs of SEQ ID NOs: 31, 32 and 33. In some embodiments, an anti-CD47 antibody comprises heavy chain CDRs of SEQ ID NOs: 28, 29 and 30 and/or light chain CDRs of SEQ ID NOs: 35, 36 and 37. In other embodiments, an anti-CD47 antibody comprises the HCVR/LCVR pair SEQ ID NO: 22/SEQ ID NO: 23. In other embodiments, an anti-CD47 antibody comprises a HCVR that has at least about 80% homology or identity (e.g., 80%, 85%, 90%, 91%, 92%, 93%, 94%, 95%, 96%, 97%, 98% or 99%) to SEQ ID NO: 22 and/or comprises a LCVR that has at least about 80% homology or identity (e.g., 80%, 85%, 90%, 91%, 92%, 93%, 94%, 95%, 96%, 97%, 98% or 99%) to SEQ ID NO: 23.

### hB6H12.3 G91A

In certain exemplary embodiments, an anti-CD47 antibody comprises CDRs from a HCVR set forth as SEQ ID NO: 22 and/or CDRs from a LCVR set forth as SEQ ID NO: 24. In other embodiments, an anti-CD47 antibody comprises heavy chain CDRs of SEQ ID NOs: 25, 26 and 27 and/or light chain CDRs of SEQ ID NOs: 31, 32, and 34. In some embodiments, an anti-CD47 antibody comprises heavy chain CDRs of SEQ ID NOs: 28, 29 and 30 and/or light chain CDRs of SEQ ID NOs: 35, 36 and 38. In other embodiments, an anti-CD47 antibody comprises the HCVR/LCVR pair SEQ ID NO: 22/SEQ ID NO: 24. In other embodiments, an anti-CD47 antibody comprises a HCVR that has at least about 80% homology or identity (e.g., 80%, 85%, 90%, 91%, 92%, 93%, 94%, 95%, 96%, 97%, 98% or 99%) to SEQ ID NO: 22 and/or comprises a LCVR that has at least about 80% homology or identity (e.g., 80%, 85%, 90%, 91%, 92%, 93%, 94%, 95%, 96%, 97%, 98% or 99%) to SEQ ID NO: 24.

The anti-CD47 antibodies described herein typically bind CD47 with an equilibrium binding constant of ≤1 μM, e.g., ≤100 nM, preferably ≤10 nM, and more preferably ≤1 nM, as measured using standard binding assays, for example, the Biacore®-based binding assay.

Antibody molecules used in the present formulations may be characterized relative to a reference anti-CD47 antibody, for example, B6H12, 2D3, MABL, CC2C6, or BRIC126. Antibody B6H12 is described, for example, in U.S. Pat. Nos. 5,057,604 and 9,017,675, is commercially available from Abcam, PLC, Santa Cruz Biotechnology, Inc., and eBioscience, Inc.

### Glycosylation Variants

Antibodies may be glycosylated at conserved positions in their constant regions (Jefferis and Lund, (1997) Chem. Immunol. 65:111-128; Wright and Morrison, (1997) TibTECH 15:26-32). The oligosaccharide side chains of the immunoglobulins affect the protein's function (Boyd et al., (1996) Mol. Immunol. 32:1311-1318; Wittwe and Howard, (1990) Biochem. 29:4175-4180), and the intramolecular interaction between portions of the glycoprotein which can affect the conformation and presented three-dimensional surface of the glycoprotein (Jefferis and Lund, supra; Wyss and Wagner, (1996) Current Op. Biotech. 7:409-416). Oligosaccharides may also serve to target a given glycoprotein to certain molecules based upon specific recognition structures. For example, it has been reported that in agalactosylated IgG, the oligosaccharide moiety ‘flips’ out of the inter-CH2 space and terminal N-acetylglucosamine residues become available to bind mannose binding protein (Malhotra et al., (1995) Nature Med. 1:237-243). Removal by glycopeptidase of the oligosaccharides from CAMPATH-1H (a recombinant humanized murine monoclonal IgG1 antibody which recognizes the CDw52 antigen of human lymphocytes) produced in Chinese Hamster Ovary (CHO) cells resulted in a complete reduction in complement mediated lysis (CMCL) (Boyd et al., (1996) Mol. Immunol. 32:1311-1318), while selective removal of sialic acid residues using neuraminidase resulted in no loss of DMCL. Glycosylation of antibodies has also been reported to affect antibody-dependent cellular cytotoxicity (ADCC). In particular, CHO cells with tetracycline-regulated expression of α(1,4)-N-acetylglucosaminyltransferase III (GnTIII), a glycosyltransferase catalyzing formation of bisecting GlcNAc, was reported to have improved ADCC activity (Umana et al. (1999) Mature Biotech. 17:176-180).

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Glycosylation variants of antibodies are variants in which the glycosylation pattern of an antibody is altered. By altering is meant deleting one or more carbohydrate moieties found in the antibody, adding one or more carbohydrate moieties to the antibody, changing the composition of glycosylation (glycosylation pattern), the extent of glycosylation, etc.

Addition of glycosylation sites to an antibody can be accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites). Similarly, removal of glycosylation sites can be accomplished by amino acid alteration within the native glycosylation sites of the antibody.

The amino acid sequence is usually altered by altering the underlying nucleic acid sequence. These methods include isolation from a natural source (in the case of naturally-occurring amino acid sequence variants) or preparation by oligonucleotide-mediated (or site-directed) mutagenesis, PCR mutagenesis, and cassette mutagenesis of an earlier prepared variant or a non-variant version of the antibody.

The glycosylation (including glycosylation pattern) of antibodies may also be altered without altering the amino acid sequence or the underlying nucleotide sequence. Glycosylation largely depends on the host cell used to express the antibody. Since the cell type used for expression of recombinant glycoproteins, e.g., antibodies, as potential therapeutics is rarely the native cell, significant variations in the glycosylation pattern of the antibodies can be expected. See, e.g., Hse et al., (1997) J. Biol. Chem. 272:9062-9070. In addition to the choice of host cells, factors which affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261; 5,278,299). Glycosylation, or certain types of glycosylation, can be enzymatically removed from the glycoprotein, for example using endoglycosidase H (Endo H). In addition, the recombinant host cell can be genetically engineered, e.g., make defective in processing certain types of polysaccharides. These and similar techniques are well known in the art.

The glycosylation structure of antibodies can be readily analyzed by conventional techniques of carbohydrate analysis, including lectin chromatography, NMR, Mass spectrometry, HPLC, GPC, monosaccharide compositional analysis, sequential enzymatic digestion, and HPAEC-PAD, which uses high pH anion exchange chromatography to separate oligosaccharides based on charge. Methods for releasing oligosaccharides for analytical purposes are also known, and include, without limitation, enzymatic treatment (commonly performed using peptide-N-glycosidase F/endo-β-galactosidase), elimination using harsh alkaline environment to release mainly O-linked structures, and chemical methods using anhydrous hydrazine to release both N- and O-linked oligosaccharides.

A preferred form of modification of glycosylation of antibodies is reduced core fucosylation. “Core fucosylation” refers to addition of fucose (“fucosylation”) to N-acetylglucosamine (“GlcNAc”) at the reducing terminal of an N-linked glycan.

A “complex N-glycoside-linked sugar chain” is typically bound to asparagine 297 (according to the number of Kabat). As used herein, the complex N-glycoside-linked sugar chain has a biantennary composite sugar chain, mainly having the following structure:

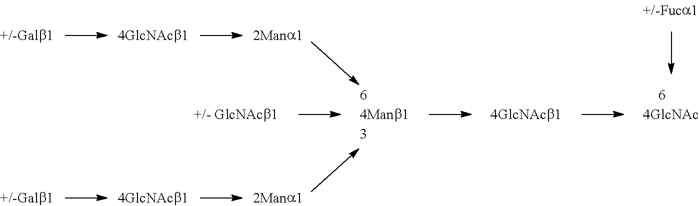

where +/− indicates the sugar molecule can be present or absent, and the numbers indicate the position of linkages between the sugar molecules. In the above structure, the sugar chain terminal which binds to asparagine is called a reducing terminal (at right), and the opposite side is called a non-reducing terminal. Fucose is usually bound to N-acetylglucosamine (“GlcNAc”) of the reducing terminal, typically by an α1,6 bond (the 6-position of GlcNAc is linked to the 1-position of fucose). “Gal” refers to galactose, and “Man” refers to mannose.

A “complex N-glycoside-linked sugar chain” includes 1) a complex type, in which the non-reducing terminal side of the core structure has one or more branches of galactose-N-acetylglucosamine (also referred to as “gal-GlcNAc”) and the non-reducing terminal side of Gal-GlcNAc optionally has a sialic acid, bisecting N-acetylglucosamine or the like; or 2) a hybrid type, in which the non-reducing terminal side of the core structure has both branches of a high mannose N-glycoside-linked sugar chain and complex N-glycoside-linked sugar chain.

In some embodiments, the “complex N-glycoside-linked sugar chain” includes a complex type in which the non-reducing terminal side of the core structure has zero, one or more branches of galactose-N-acetylglucosamine (also referred to as “gal-GlcNAc”) and the non-reducing terminal side of Gal-GlcNAc optionally further has a structure such as a sialic acid, bisecting N-acetylglucosamine or the like.

According to certain methods, only a minor amount of fucose is incorporated into the complex N-glycoside-linked sugar chain(s) of an antibody. For example, in various embodiments, less than about 60%, less than about 50%, less than about 40%, less than about 30%, less than about 20%, less than about 15%, less than about 10%, less than about 5%, or less than about 3% of the molecules of an antibody have core fucosylation by fucose. In some embodiments, about 2% of the molecules of the antibody has core fucosylation by fucose.

In certain embodiments, only a minor amount of a fucose analog (or a metabolite or product of the fucose analog) is incorporated into the complex N-glycoside-linked sugar chain(s). For example, in various embodiments, less than about 60%, less than about 50%, less than about 40%, less than about 30%, less than about 20%, less than about 15%, less than about 10%, less than about 5%, or less than about 3% of the antibodies have core fucosylation by a fucose analog or a metabolite or product of the fucose analog. In some embodiments, about 2% of the antibodies have core fucosylation by a fucose analog or a metabolite or product of the fucose analog.

Methods of making non-fucosylated antibodies (which may be used to make non-fucosylated masked antibodies) by incubating antibody-producing cells with a fucose analogue are described, e.g., in WO2009/135181. Briefly, cells that have been engineered to express the antibody are incubated in the presence of a fucose analogue or an intracellular metabolite or product of the fucose analog. An intracellular metabolite can be, for example, a GDP-modified analog or a fully or partially de-esterified analog. A product can be, for example, a fully or partially de-esterified analog. In some embodiments, a fucose analogue can inhibit an enzyme(s) in the fucose salvage pathway. For example, a fucose analog (or an intracellular metabolite or product of the fucose analog) can inhibit the activity of fucokinase, or GDP-fucose-pyrophosphorylase. In some embodiments, a fucose analog (or an intracellular metabolite or product of the fucose analog) inhibits fucosyltransferase (preferably a 1,6-fucosyltransferase, e.g., the FUT8 protein). In some embodiments, a fucose analog (or an intracellular metabolite or product of the fucose analog) can inhibit the activity of an enzyme in the de novo synthetic pathway for fucose. For example, a fucose analog (or an intracellular metabolite or product of the fucose analog) can inhibit the activity of GDP-mannose 4,6-dehydratase or/or GDP-fucose synthetase. In some embodiments, the fucose analog (or an intracellular metabolite or product of the fucose analog) can inhibit a fucose transporter (e.g., GDP-fucose transporter).

In one embodiment, the fucose analogue is 2-flurofucose. Methods of using fucose analogues in growth medium and other fucose analogues are disclosed, e.g., in WO/2009/135181, which is herein incorporated by reference.

Other methods for engineering cell lines to reduce core fucosylation included gene knock-outs, gene knock-ins and RNA interference (RNAi). In gene knock-outs, the gene encoding FUT8 (alpha 1,6-fucosyltransferase enzyme) is inactivated. FUT8 catalyzes the transfer of a fucosyl residue from GDP-fucose to position 6 of Asn-linked (N-linked) GlcNac of an N-glycan. FUT8 is reported to be the only enzyme responsible for adding fucose to the N-linked biantennary carbohydrate at Asn297. Gene knock-ins add genes encoding enzymes such as GNTIII or a Golgi alpha mannosidase II. An increase in the levels of such enzymes in cells diverts monoclonal antibodies from the fucosylation pathway (leading to decreased core fucosylation), and having increased amount of bisecting N-acetylglucosamines. RNAi typically also targets FUT8 gene expression, leading to decreased mRNA transcript levels or knocking out gene expression entirely. Any of these methods can be used to generate a cell line that would be able to produce a non-fucosylated antibody.

Many methods are available to determine the amount of fucosylation on an antibody. Methods include, e.g., LC-MS via PLRP-S chromatography and electrospray ionization quadrupole TOF MS.

### IV. Linking Coiled Coil Masking Agents to Antibodies

Coiled coil forming peptides are linked to the amino-termini of antibody variable regions via a linker including a protease site. A typical antibody includes a heavy and light chain variable region, in which case a coiled coil forming peptide is linked to the amino-termini of each. A bivalent antibody has two binding sites, which may or may not be the same. In a normal monospecific antibody, the binding sites are the same and the antibody has two identical light and heavy chain pairs. In this case, each heavy chain is linked to the same coiled coil forming peptide and each light chain to the same coiled coil forming peptide (which may or may not be the same as the peptide linked to the heavy chain). In a bispecific antibody, the binding sites are different and formed from two different heavy and light chain pairs. In such a case, the heavy and light chain variable region of one binding site are respectively linked to coiled coil forming peptides as are the heavy and light chain variable regions of the other binding site. Typically both heavy chain variable regions are linked to the same type of coiled coil forming peptide as are both light chain variable regions.

A coiled coil-forming peptide can be linked to an antibody variable region via a linker including a protease site. Typically, the same linker with the same protease cleavage site is used for linking each heavy or light chain variable region of an antibody to a coiled coil peptide. The protease cleavage site should be one amenable to cleavage by a protease present extracellularly in the intended target tissue or pathology, such as a cancer, such that cleavage of the linker releases the antibody from the coiled coil masking its activity allowing the antibody to bind to its intended target, such as a cell-surface antigen or soluble ligand.

As well as the variable regions, a masked antibody typically includes all or part of a constant region, which can include any or all of a light chain constant region, CH1, hinge, CH2 and CH3 regions. As with other antibodies one or more carboxy-terminal residues can be proteolytically processed or derivatized.

Coiled coils can be formed from the same peptide forming a homodimer or two different peptides forming a heterodimer. For formation of a homodimer, light and heavy antibody chains are linked to the same coiled coil forming peptide. For formation of a heterodimer, light and heavy antibody chains are linked to different coiled coils peptides. For some pairs of coiled coil forming peptides, it is preferred that one of the pair be linked to the heavy chain and the other to the light chain of an antibody although the reverse orientation is also possible.

Each antibody chain can be linked to a single coiled coil forming peptide or multiple such peptides in tandem (e.g., two, three, four or five copies of a peptide). If the latter, the peptides in tandem linkage are usually the same. Also if tandem linkage is employed, light and heavy chains are usually linked to the same number of peptides.

Linkage of antibody chains to coiled coil forming peptides can reduce the binding affinity of an antibody by at least about 10-fold, at least about 50-fold, at least about 100-fold, at least about 200-fold, at least about 500-fold, at least about 1000-fold or at least about 1500-fold relative to the same antibody without such linkage or after cleavage of such linkage. In some such antibodies, binding affinity is reduced between about between about 50-5000-fold, 50-1500-fold, between about 100-1500-fold, between about 200-1500-fold, between about 500-1500-fold, between about 50-5000-fold, between about 50-1000-fold, between about 100-1000-fold, between about 200-1000-fold, between about 500-1000-fold, between about 50-500-fold, or between about 100-500-fold. Effector functions of the antibody, such as ADCC, phagocytosis, and CDC or cytotoxicity as a result of linkage to a drug in an antibody drug conjugate can be reduced by the same factors or ranges. Upon proteolytic cleavage that serves to unmask an antibody or otherwise remove the mask from the antibody, the restored antibody typically has an affinity or effect function that is within a factor of 2, 1.5 or preferably unchanged within experimental error compared with an otherwise identical control antibody, which has never been masked.

### V. Antibody-Drug Conjugates

In certain embodiments, a masked antibody may comprise an antibody drug conjugates (ADCs, also referred to herein as an “immunoconjugate”). Particular ADCs may comprise cytotoxic agents (e.g., chemotherapeutic agents), prodrug converting enzymes, radioactive isotopes or compounds, or toxins (these moieties being collectively referred to as a therapeutic agent). For example, an ADC can be conjugated to a cytotoxic agent such as a chemotherapeutic agent, or a toxin (e.g., a cytostatic or cytocidal agent such as, for example, abrin, ricin A, pseudomonas exotoxin, or diphtheria toxin). Examples of useful classes of cytotoxic agents include, for example, DNA minor groove binders, DNA replication inhibitors, DNA alkylating agents, NAMPT inhibitors, and tubulin inhibitors (i.e., antitubulins). Exemplary cytotoxic agents include, for example, auristatins, camptothecins, calicheamicins, duocarmycins, etoposides, enediyine antibiotics, maytansinoids (e.g., DM1, DM2, DM3, DM4), taxanes, benzodiazepines (e.g., pyrrolo[1,4]benzodiazepines, indolinobenzodiazepines, and oxazolidinobenzodiazepines including pyrrolo[1,4]benzodiazepine dimers, indolinobenzodiazepine dimers, and oxazolidinobenzodiazepine dimers), lexitropsins, taxanes, combretastatins, cryptophysins, and vinca alkaloids. Nonlimiting exemplary cytotoxig agents include auristatin E, AFP, AEB, AEVB, MMAF, MMAE, paclitaxel, docetaxel, doxorubicin, morpholino-doxorubicin, cyanomorpholino-doxorubicin, melphalan, methotrexate, mitomycin C, a CC-1065 analogue, CBI, calicheamicin, maytansine, an analog of dolastatin 10, rhizoxin, or palytoxin, epothilone A, epothilone B, nocodazole, colchicine, colcimid, estramustine, cemadotin, discodermolide, eleutherobin, a tubulysin, a plocabulin, and maytansine.

An ADC can be conjugated to a pro-drug converting enzyme. The pro-drug converting enzyme can be recombinantly fused to the antibody or chemically conjugated thereto using known methods. Exemplary pro-drug converting enzymes are carboxypeptidase G2, beta-glucuronidase, penicillin-V-amidase, penicillin-G-amidase, β-lactamase, β-glucosidase, nitroreductase and carboxypeptidase A.

Techniques for conjugating therapeutic agents to proteins, and in particular to antibodies, are well-known. (See, e.g., Alley et al., Current Opinion in Chemical Biology 2010 14: 1-9; Senter, Cancer J., 2008, 14 (3): 154-169.) The therapeutic agent can be conjugated in a manner that reduces its activity unless it is cleaved off the antibody (e.g., by hydrolysis, by proteolytic degradation, or by a cleaving agent). In some aspects, the therapeutic agent is attached to the antibody with a cleavable linker that is sensitive to cleavage in the intracellular environment of the antigen-expressing cancer cell but is not substantially sensitive to the extracellular environment, such that the conjugate is cleaved from the antibody when it is internalized by the antigen-expressing cancer cell (e.g., in the endosomal or, for example by virtue of pH sensitivity or protease sensitivity, in the lysosomal environment or in the caveolear environment). In some embodiments, the therapeutic agent can also be attached to the antibody with a non-cleavable linker.

In certain exemplary embodiments, an ADC can include a linker region between a cytotoxic or cytostatic agent and the antibody. As noted supra, typically, the linker can be cleavable under intracellular conditions, such that cleavage of the linker releases the therapeutic agent from the antibody in the intracellular environment (e.g., within a lysosome or endosome or caveolea). The linker can be, e.g., a peptidyl linker that is cleaved by an intracellular peptidase or protease enzyme, including a lysosomal or endosomal protease. Cleaving agents can include cathepsins B and D and plasmin (see, e.g., Dubowchik and Walker, Pharm. Therapeutics 83:67-123, 1999). Most typical are peptidyl linkers that are cleavable by enzymes that are present in antigen-expressing cells. For example, a peptidyl linker that is cleavable by the thiol-dependent protease cathepsin-B, which is highly expressed in cancerous tissue, can be used (e.g., a linker comprising a Phe-Leu or a Val-Cit peptide).

A cleavable linker can be pH-sensitive, i.e., sensitive to hydrolysis at certain pH values. Typically, the pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, Pharm. Therapeutics 83:67-123, 1999; Neville et al, Biol. Chem. 264: 14653-14661, 1989.) Such linkers are relatively stable under neutral pH conditions, such as those in the blood, but are unstable at below pH 5.5 or 5.0, the approximate pH of the lysosome.

Other linkers are cleavable under reducing conditions (e.g., a disulfide linker). Disulfide linkers include those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT. (See, e.g., Thorpe et al., Cancer Res. 47:5924-5931, 1987; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

The linker can also be a malonate linker (Johnson et al, Anticancer Res. 15: 1387-93, 1995), a maleimidobenzoyl linker (Lau et al., Bioorg-Med-Chem. 3: 1299-1304, 1995), or a 3′-N-amide analog (Lau et al., Bioorg-Med-Chem. 3: 1305-12, 1995).

The linker also can be a non-cleavable linker, such as an maleimido-alkylene or maleimide-aryl linker that is directly attached to the therapeutic agent and released by proteolytic degradation of the antibody.

Typically, the linker is not substantially sensitive to the extracellular environment, meaning that no more than about 20%, typically no more than about 15%, more typically no more than about 10%, and even more typically no more than about 5%, no more than about 3%, or no more than about 1% of the linkers in a sample of the ADC is cleaved when the ADC is present in an extracellular environment (e.g., in plasma). Whether a linker is not substantially sensitive to the extracellular environment can be determined, for example, by incubating independently with plasma both (a) the ADC (the “ADC sample”) and (b) an equal molar amount of unconjugated antibody or therapeutic agent (the “control sample”) for a predetermined time period (e.g., 2, 4, 8, 16, or 24 hours) and then comparing the amount of unconjugated antibody or therapeutic agent present in the ADC sample with that present in control sample, as measured, for example, by high performance liquid chromatography.

The linker can also promote cellular internalization. The linker can promote cellular internalization when conjugated to the therapeutic agent (i.e., in the milieu of the linker-therapeutic agent moiety of the ADC or ADC derivate as described herein). Alternatively, the linker can promote cellular internalization when conjugated to both the therapeutic agent and the antibody (i.e., in the milieu of the ADC as described herein).

The antibody can be conjugated to the linker via a heteroatom of the antibody. These heteroatoms can be present on the antibody in its natural state or can be introduced into the antibody. In some aspects, the antibody will be conjugated to the linker via a nitrogen atom of a lysine residue. In other aspects, the antibody will be conjugated to the linker via a sulfur atom of a cysteine residue. Methods of conjugating linker and drug-linkers to antibodies are known in the art.

Exemplary antibody-drug conjugates include auristatin based antibody-drug conjugates meaning that the drug component is an auristatin drug. Auristatins bind tubulin, have been shown to interfere with microtubule dynamics and nuclear and cellular division, and have anticancer activity. Typically the auristatin based antibody-drug conjugate comprises a linker between the auristatin drug and the antibody. The linker can be, for example, a cleavable linker (e.g., a peptidyl linker) or a non-cleavable linker (e.g., linker released by degradation of the antibody). Auristatins include MMAF, and MMAE. The synthesis and structure of exemplary auristatins are described in U.S. Publication Nos. 7,659,241, 7,498,298, 2009-0111756, 2009-0018086, and 7,968, 687 each of which is incorporated herein by reference in its entirety and for all purposes.

Other exemplary antibody-drug conjugates include maytansinoid antibody-drug conjugates meaning that the drug component is a maytansinoid drug, and benzodiazepine antibody drug conjugates meaning that the drug component is a benzodiazepine (e.g., pyrrolo[1,4]benzodiazepine dimers, indolinobenzodiazepine dimers, and oxazolidinobenzodiazepine dimers).

In certain embodiments, an antibody may be combined with an ADC with binding specificity to a different target. Exemplary ADCs that may be combined with a masked antibody include brentuximab vedotin (anti-CD30 ADC), enfortumab vedotin (anti-nectin-4 ADC), ladiratuzumab vedotin (anti-LIV-1 ADC), denintuzumab mafodotin (anti-CD19 ADC), glembatumumab vedotin (anti-GPNMB ADC), anti-TIM-1 ADC, polatuzumab vedotin (anti-CD79b ADC), anti-MUC16 ADC, depatuxizumab mafodotin, telisotuzumab vedotin, anti-PSMA ADC, anti-C4.4a ADC, anti-BCMA ADC, anti-AXL ADC, tisotuumab vedotin (anti-tissue factor ADC).

### VI. Masked Antibody Expression

Nucleic acids encoding masked antibodies can be expressed in a host cell that contains endogenous DNA encoding a masked antibody used in the present invention. Such methods are well known in the art, e.g., as described in U.S. Pat. Nos. 5,580,734, 5,641,670, 5,733,746, and 5,733,761. Also see, e.g., Sambrook, et al., supra, and Ausubel, et al., supra. Those of ordinary skill in the art are knowledgeable in the numerous expression systems available for expression of a nucleic acid encoding a protein of the present invention. Illustrative of cell cultures useful for the production of the antibodies, masked antibodies, specified portions or variants thereof, are mammalian cells. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, hep G2 cells, P3X63Ag8.653, SP2/0-Ag14, HeLa cells and the like, which are readily available from, for example, American Type Culture Collection, Manassas, Va. Yeast and bacterial host cells may also be used and are well known to those of skill in the art. Other cells useful for production of nucleic acids or proteins of the present invention are known and/or available, for instance, from the American Type Culture Collection Catalogue of Cell Lines and hybridomas or other known or commercial sources.

Expression vectors can include one or more of the following expression control sequences, such as, but not limited to an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences). See, e.g., Ausubel et al., supra; Sambrook, et al., supra.

Expression vectors optionally include at least one selectable marker. Such markers include, e.g., but are not limited to, methotrexate (MTX), dihydrofolate reductase (DHFR, U.S. Pat. Nos. 4,399,216; 4,634,665; 4,656,134; 4,956,288; 5,149,636; 5,179,017), ampicillin, neomycin (G418), mycophenolic acid, or glutamine synthetase (GS, U.S. Pat. Nos. 5,122,464; 5,770,359; and 5,827,739), resistance for eukaryotic cell culture, and tetracycline or ampicillin resistance genes for culturing in E. coli and other bacteria or prokaryotes. Appropriate culture media and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra; Ausubel, supra.

The nucleic acid insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

The nucleic acid insert is optionally in frame with a coiled coil sequence and/or an MMP cleavage sequence, e.g., at the amino-terminus of one or more heavy chain and/or light chain sequences. Alternatively, a coiled coil sequence and/or an MMP cleavage sequence can be post-translationally added to an antibody, e.g., via a disulfide bond or the like.

When eukaryotic host cells are employed, polyadenylation or transcription terminator sequences are typically incorporated into the vector. An example of a terminator sequence is the polyadenylation sequence from the bovine growth hormone gene. Sequences for accurate splicing of the transcript can also be included. An example of a splicing sequence is the VP1 intron from SV40 (Sprague, et al. (1983) J. Virol. 45:773-781). Additionally, gene sequences to control replication in the host cell can be incorporated into the vector, as known in the art.

### VII. Masked Antibody Isolation and Purification

Masked antibodies used in the present formulations can be recovered and purified from recombinant cell cultures by methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (HPLC) can also be employed for purification. See, e.g., Colligan, Current Protocols in Immunology, or Current Protocols in Protein Science, John Wiley & Sons, New York, N.Y., (1997-2001).

In some embodiments, antibodies or masked antibodies described herein can be expressed in a modified form. For instance, a region of additional amino acids, particularly charged amino acids, can be added to the amino-terminus of an antibody to improve stability and persistence in the host cell, during purification, or during subsequent handling and storage. Also, peptide moieties can be added to an antibody or masked antibody to facilitate purification. Such regions can be removed prior to final preparation of an antibody or masked antibody. Such methods are described in many standard laboratory manuals, such as Sambrook, supra; Ausubel, et al., ed., Current Protocols In Molecular Biology, John Wiley & Sons, Inc., NY, N.Y. (1987-2001).

Antibodies and masked antibodies described herein can include purified products, products of chemical synthetic procedures, and products produced by recombinant techniques from a eukaryotic host, including, for example, yeast, higher plant, insect and mammalian cells. Depending upon the host employed in a recombinant production procedure, the antibody or masked antibody of the present invention can be glycosylated or can be non-glycosylated, with glycosylated preferred. Such methods are described in many standard laboratory manuals, such as Sambrook, supra; Ausubel, supra, Colligan, Protein Science, supra.

In some embodiments, methods of determining the amount of demasked antibody in an aqueous or lyophilized formulation are provided. In some embodiments, the lyophilized formulation is reconstituted, such as in water, to form a reconstituted aqueous formulation prior to determining the amount of demasked antibody in the lyophilized formulation. In some embodiments, determining the amount of demasked antibody in an aqueous formulation or reconstituted aqueous formulation is carried out using Capillary Electrophoresis with Sodium Dodecyl Sulfate (CE-SDS). A nonlimiting exemplary method using CE-SDS is described in Example 10. Briefly, SDS and a reducing agent, such as DTT, are added to a sample, for example, by diluting in tris buffer comprising SDS and the reducing agent (in some instances, to a final concentration of 5 mM, such as 5 mM DTT), and the sample is alkylated with iodoacetamide. A nonlimiting exemplary method of alkylating the sample is described in Salas-Solano et al., Anal. Chem. 2006, 78: 6583-6594. In some embodiments, the sample is then separated on a capillary electrophoresis system, such as a capillary electrophoresis system containing a bare fused-silica capillary filled with SDS gel buffer, for example, at a voltage of 15.0 kV for 30 minutes with a capillary temperature of 25° C. Material is detected by UV at 220 nm. In some embodiments, the data may be analyzed using Empower 3 CDS software. In some embodiments, demasked light chain is detected in the Pre-L region in the electropherogram, which is prior to the region where the masked light chain is detected. In some embodiments, the amount of demasked antibody in the sample may be calculated based on the peak area of the demasked light chain in the PreL region.

In some embodiments, a quality control standard is applied such that a sample of masked antibody passes the quality control standard if the peak area in the PreL region is less than 0.8%, or less than 0.7%, or less than 0.6%, or less than 0.5%, or less than 0.4%. In some embodiments, a sample of masked antibody passes the quality control standard if the peak area in the PreL region is less than 0.6%.

In some embodiments, a quality control standard is applied such that a sample of masked antibody passes the quality control standard if the amount of masked antibody that is demasked in the sample, for example, as calculated based on the peak area in the PreL region, is less than 2%, less than 1.9%, less than 1.8%, less than 1.7%, less than 1.6%, or less than 1.5%. In some embodiments, a quality control standard is applied such that a sample of masked antibody passes the quality control standard if the amount of masked antibody that is demasked in the sample, for example, as calculated based on the peak area in the PreL region, is less than 1.7%.

### VIII. Therapeutic Applications

In some embodiments, formulations herein may be used in methods of therapeutic treatment. Nonlimiting exemplary diseases and disorders that may be treated with the formulations provided herein include cancer, autoimmune disorders, and infections. Where the masked antibodies comprise anti-CD47 antibodies, for example, the formulations herein may be used for methods of treating disorders associated with cells that express CD47, e.g., cancers. The cells may or may not express elevated levels of CD47 relative to cells that are not associated with a disorder of interest. As a result, the formulations may be used in a method of treating a subject, for example, a subject with a cancer, using the masked anti-CD47 antibodies described herein. The methods comprise administering an effective amount of a masked anti-CD47 antibody or a composition comprising a masked anti-CD47 antibody to a subject in need thereof

Positive therapeutic effects in cancer can be measured in a number of ways (See, W. A. Weber, J. Null. Med. 50:1S-10S (2009); Eisenhauer et al., supra). In some preferred embodiments, response to a masked antibody is assessed using RECIST 1.1 criteria. In some embodiments, the treatment achieved by a therapeutically effective amount is any of a partial response (PR), a complete response (CR), progression free survival (PFS), disease free survival (DFS), objective response (OR) or overall survival (OS). The dosage regimen of a therapy described herein that is effective to treat a primary or a secondary hepatic cancer patient may vary according to factors such as the disease state, age, and weight of the patient, and the ability of the therapy to elicit an anti-cancer response in the subject. While an embodiment of the treatment method, medicaments and uses of the present invention may not be effective in achieving a positive therapeutic effect in every subject, it should do so in a statistically significant number of subjects as determined by any statistical test known in the art such as the Student's t-test, the chi2-test, the U-test according to Mann and Whitney, the Kruskal-Wallis test (H-test), Jonckheere-Terpstra-test and the Wilcoxon-test.

“RECIST 1.1 Response Criteria” as used herein means the definitions set forth in Eisenhauer et al., E. A. et al., Eur. J Cancer 45:228-247 (2009) for target lesions or non-target lesions, as appropriate, based on the context in which response is being measured.

“Tumor” as it applies to a subject diagnosed with, or suspected of having, a primary or a secondary hepatic cancer, refers to a malignant or potentially malignant neoplasm or tissue mass of any size. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms). Nonlimiting exemplary sarcomas include soft tissue sarcoma and osteosarcoma.

“Tumor burden” also referred to as “tumor load,” refers to the total amount of tumor material distributed throughout the body. Tumor burden refers to the total number of cancer cells or the total size of tumor(s) throughout the body, including lymph nodes and bone narrow. Tumor burden can be determined by a variety of methods known in the art, such as, e.g., by measuring the dimensions of tumor(s) upon removal from the subject, e.g., using calipers, or while in the body using imaging techniques, e.g., ultrasound, bone scan, computed tomography (CT) or magnetic resonance imaging (MM) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

Nonlimiting exemplary autoimmune diseases that may be treated with a masked antibody include Crohn's disease, ulcerative colitis, rheumatoid arthritis, psoriatic arthritis, ankylosing spondylitis, uveitis, juvenile idiopathic arthritis, multiple sclerosis, psoriasis (including plaque psoriasis), systemic lupus erythematosus, granulomatosis with polyangiitis, microscopic polyangiitis, systemic sclerosis, idiopathic thrombocytopenic purpura, graft-versus-host disease, and autoimmune cytopenias.

As used herein, the term “effective amount” refers to the amount of a compound (e.g., a masked antibody) sufficient to effect beneficial or desired results. An effective amount can be administered in one or more administrations, applications or dosages and is not intended to be limited to a particular formulation or administration route. Generally, a therapeutically effective amount of active component is in the range of 0.01 mg/kg to 100 mg/kg, 0.1 mg/kg to 100 mg/kg, 1 mg/kg to 100 mg/kg, 0.01 mg/kg to 10 mg/kg, 0.1 mg/kg to 10 mg/kg, 1 mg/kg to 10 mg/kg. The dosage administered can vary depending upon known factors, such as the pharmacodynamic characteristics of the particular agent, and its mode and route of administration; the age, health, and weight of the recipient; the type and extent of disease or indication to be treated, the nature and extent of symptoms, kind of concurrent treatment, frequency of treatment, and the effect desired. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the antibody, and the disease being treated. Exemplary dosing frequencies are once per day, once per week and once every two weeks.

In certain exemplary embodiments, the present invention provides a method for treating cancer in a cell, tissue, organ, animal or patient. In particular embodiments, the present invention provides a method for treating a solid cancer in a human. Examples of cancers include, but are not limited to, solid tumors, soft tissue tumors, hematopoietic tumors that give rise to solid tumors, and metastatic lesions. Examples of hematopoietic tumors that have the potential to give rise to solid tumors include, but are not limited to, diffuse large B-cell lymphomas (DLBCL), follicular lymphoma, myelodysplastic syndrome (MDS), a lymphoma, Hodgkin's disease, a malignant lymphoma, non-Hodgkin's lymphoma, Burkitt's lymphoma, multiple myeloma, Richter's Syndrome (Richter's Transformation) and the like. Examples of solid tumors include, but are not limited to, malignancies, e.g., sarcomas (including soft tissue sarcoma and osteosarcoma), adenocarcinomas, and carcinomas, of the various organ systems, such as those affecting head and neck (including pharynx), thyroid, lung (small cell or non-small cell lung carcinoma (NSCLC)), breast, lymphoid, gastrointestinal tract (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), central nervous system (e.g., neural or glial cells, e.g., neuroblastoma or glioma), skin (e.g., melanoma) and the like. In certain embodiments, the solid tumor is an NMDA receptor positive teratoma. In other embodiments, the cancer is selected from breast cancer, colon cancer, pancreatic cancer (e.g., a pancreatic neuroendocrine tumors (PNET) or a pancreatic ductal adenocarcinoma (PDAC)), stomach cancer, uterine cancer, and ovarian cancer. In some embodiments, the cancer expresses CD47, and is treated with a masked anti-CD47 antibody.

In certain embodiments, the cancer is selected from, but not limited to, leukemia's such as acute lymphoblastic leukemia (ALL), chronic lymphocytic leukemia (CLL), acute myelogenous leukemia (AML), chronic myelogenous leukemia (CIVIL), hairy cell leukemia (HCL), T-cell prolymphocytic leukemia (T-PLL), large granular lymphocytic leukemia, adult T-cell leukemia, and acute monocytic leukemia (AMoL).

In one embodiment, the cancer is a solid tumor that is associated with ascites. Ascites is a symptom of many types of cancer and can also be caused by a number of conditions, such as advanced liver disease. The types of cancer that are likely to cause ascites include, but are not limited to, cancer of the breast, lung, large bowel (colon), stomach, pancreas, ovary, uterus (endometrium), peritoneum and the like. In some embodiments, the solid tumor associated with ascites is selected from breast cancer, colon cancer, pancreatic cancer, stomach, uterine cancer, and ovarian cancer. In some embodiments, the cancer is associated with pleural effusions, e.g., lung cancer.

Additional hematological cancers that give rise to solid tumors include, but are not limited to, non-Hodgkin lymphoma (e.g., diffuse large B cell lymphoma, mantle cell lymphoma, B lymphoblastic lymphoma, peripheral T cell lymphoma and Burkitt's lymphoma), B-lymphoblastic lymphoma; B-cell chronic lymphocytic leukemia/small lymphocytic lymphoma; lymphoplasmacytic lymphoma; splenic marginal zone B-cell lymphoma (±villous lymphocytes); plasma cell myeloma/plasmacytoma; extranodal marginal zone B-cell lymphoma of the MALT type; nodal marginal zone B-cell lymphoma (±monocytoid B cells); follicular lymphoma; diffuse large B-cell lymphomas; Burkitt's lymphoma; precursor T-lymphoblastic lymphoma; T adult T-cell lymphoma (HTLV 1-positive); extranodal NK/T-cell lymphoma, nasal type; enteropathy-type T-cell lymphoma; hepatosplenic γ-δ T-cell lymphoma; subcutaneous panniculitis-like T-cell lymphoma; mycosis fungoides/sezary syndrome; anaplastic large cell lymphoma, T/null cell, primary cutaneous type; anaplastic large cell lymphoma, T-/null-cell, primary systemic type; peripheral T-cell lymphoma, not otherwise characterized; angioimmunoblastic T-cell lymphoma, multiple myeloma, polycythemia vera or myelofibrosis, cutaneous T-cell lymphoma, small lymphocytic lymphoma (SLL), marginal zone lymphoma, CNS lymphoma, immunoblastic large cell lymphoma, precursor B-lymphoblastic lymphoma and the like.

In particular embodiments, the cancer is sarcoma, colorectal cancer, head and neck cancer, lung cancer, ovarian cancer, pancreatic cancer, gastric cancer, melanoma, and/or breast cancer.

Anti-CD47 antibodies and associated masked antibodies as described herein can also be used to treat disorders associated with cancer, e.g., cancer-induced encephalopathy.

Formulations of the invention can be used in methods of treatment in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive (i.e., a synergistic response). The delivery can be such that an effect of the first treatment delivered is still detectable when the second is delivered.

In one embodiment, the methods of the invention include administering to the subject a formulation comprising a masked antibody as described herein, e.g., in combination with one or more additional therapies, e.g., surgery or administration of another therapeutic preparation. In one embodiment, in the case of cancer, for example, the additional therapy may include chemotherapy, e.g., a cytotoxic agent. In one embodiment the additional therapy may include a targeted therapy, e.g. a tyrosine kinase inhibitor, a proteasome inhibitor, or a protease inhibitor. In one embodiment, the additional therapy may include an anti-inflammatory, anti-angiogenic, anti-fibrotic, or anti-proliferative compound, e.g., a steroid, a biologic immunomodulatory, such as an inhibitor of an immune checkpoint molecule, a monoclonal antibody, an antibody fragment, an aptamer, an siRNA, an antisense molecule, a fusion protein, a cytokine, a cytokine receptor, a bronchodilator, a statin, an anti-inflammatory agent (e.g. methotrexate), or an NSAID. In another embodiment, the additional therapy could include combining therapeutics of different classes. The antibody or masked antibody preparation and the additional therapy can be administered simultaneously or sequentially.

An “immune checkpoint molecule,” as used herein, refers to a molecule in the immune system that either turns up a signal (a stimulatory molecule) or turns down a signal (an inhibitory molecule). Many cancers evade the immune system by inhibiting T cell signaling. Hence, these molecules may be used in cancer treatments as additional therapeutics. In other cases, a masked antibody may be an immune checkpoint molecule.

Exemplary immune checkpoint molecules include, but are not limited to, programmed cell death protein 1 (PD-1), programmed death-ligand 1 (PD-L1), PD-L2, cytotoxic T lymphocyte-associated protein 4 (CTLA-4), T cell immunoglobulin and mucin domain containing 3 (TIM-3), lymphocyte activation gene 3 (LAG-3), carcinoembryonic antigen related cell adhesion molecule 1 (CEACAM-1), CEACAM-5, V-domain Ig suppressor of T cell activation (VISTA), B and T lymphocyte attenuator (BTLA), T cell immunoreceptor with Ig and ITIM domains (TIGIT), leukocyte-associated immunoglobulin-like receptor 1 (LAIR1), CD160, TGFR, adenosine 2A receptor (A2AR), B7-H3 (also known as CD276), B7-H4 (also called VTCN1), indoleamine 2,3-dioxygenase (IDO), 2B4, killer cell immunoglobulin-like receptor (KIR), and the like.

An “immune checkpoint inhibitor,” as used herein, refers to a molecule (e.g., a small molecule, a monoclonal antibody, an antibody fragment, etc.) that inhibit and/or block one or more inhibitory checkpoint molecules.

Exemplary immune checkpoint inhibitors include, but are not limited to, the following monoclonal antibodies: PD-1 inhibitors such as pembrolizumab (Keytruda, Merck) and nivolumab (Opdivo, Bristol-Myers Squibb); PD-L1 inhibitors such as atezolizumab (Tecentriq, Genentech), avelumab (Bavencio, Pfizer), durvalumab (Imfinzi, AstraZeneca); and CTLA-1 inhibitors such as ipilimumab (Yervoy, Bristol-Myers Squibb).

Exemplary cytotoxic agents include anti-microtubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAHA, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent. In one embodiment, the cytotoxic agent that can be administered with a preparation described herein is a platinum-based agent (such as cisplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, etoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, 1-dehydrotestosterone, glucocorticoids, procaine, tetracaine, lidocaine, propranolol, puromycin, ricin, or maytansinoids.

Formulations of anti-CD47 antibodies or masked antibodies of the invention can be used in the treatment of subjects with CD47 positive cancer. In one embodiment, the CD47 positive cancer expresses one or more Matrix Metalloproteinases (MMPs). Exemplary MMPs include, but are not limited to, MMP1 through MMP28. Particularly exemplary MMPs include MMP2 and MMP9. In one embodiment, the CD47 positive cancer is a tumor in which infiltrating macrophages are present.

The formulations of the invention can be used in the treatment of subjects with a CD47 positive cancer that expresses one or more MMPs and contains infiltrating macrophages.

Methods of determining the presence of CD47 positive cancers, MMP expression, and the presence of tumor infiltrating macrophages are known in the art.

Assessment of CD47 positive cancers in a subject can be determined by conventional methods that include immunohistochemistry (IHC), Western blot, flow cytometry, or RNA sequencing methods. IHC, Western blot, and flow cytometry may be analyzed with any anti-CD47 antibody know in the art, as well as the anti-CD47 antibodies disclosed herein.

Assessment of macrophage infiltration in tissues can be conducted by monitoring for surface markers of macrophages, including F4/80 for mouse macrophages or CD163, CD68, or CD11b by conventional methods that include immunohistochemistry (IHC), Western blot, flow cytometry, or RNA sequencing methods.

Assessment of proteases in tissues can be monitored using a variety of techniques, including both those that monitor protease activity as well as those that can detect proteolytic activity. Conventional methods that can detect the presence of proteases in a tissue, which could include both inactive and active forms of the protease, include IHC, RNA sequencing, Western blot, or ELISA-based methods. Additional techniques can be used to detect protease activity in tissues, which includes zymography, in situ zymography by fluorescence microscopy, or the use of fluorescent proteolytic substrates. In addition, the use of fluorescent proteolytic substrates can be combined with immuno-capture of specific proteases. Additionally, antibodies directed against the active site of a protease can be used by a variety of techniques including IHC, fluorescence microscopy, Western blotting, ELISA, or flow cytometry (See, Sela-Passwell et al. Nature Medicine. 18:143-147. 2012; LeBeau et al. Cancer Research. 75:1225-1235. 2015; Sun et al. Biochemistry. 42:892-900. 2003; Shiryaev et al. 2:e80. 2013.)

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing and method steps.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting. All patents, patent applications and references described herein are incorporated by reference in their entireties for all purposes.

### EXAMPLES

### Example 1

### Stability of an Anti-CD47 Masked Antibody Vel-IPV-hB6H12.3 in Formulations With Different pH

The pH dependence of aggregation was evaluated in formulations of a masked antibody against CD47, Vel-IPV-hB6H12.3, also called “CD47M” herein (heavy chain and light chains having SEQ ID NOs: 39 and 42, respectively). An increase in the percentage of high molecular weight (HMW) antibody species over time suggests aggregation is occurring in a given formulation.

Vel-IPV-hB6H12.3 was buffer exchanged via dialysis into the following formulations (each pH condition was studied with and without 150 mM sodium chloride): 20 mM acetate pH 4, 20 mM histidine pH 5, 20 mM histidine pH 6, 20 mM potassium phosphate pH 7, and 20 mM potassium phosphate pH 8. Samples were diluted to approximately 5 mg/mL with the appropriate buffer, filled into glass vials and stored at 25° C. until the indicated time points. Analysis was performed by size-exclusion ultra performance liquid chromatography (SE-UPLC), as follows.

SE-UPLC analysis was used to measure high molecular weight (HMW), main peak (MP), and low molecular weight (LMW) forms of Vel-IPV-hB6H12.3. For SE-UPLC analysis, size distribution of Vel-IPV-hB6H12.3 was achieved using ACQUITY Protein BEH SEC Column (4.6×300 mm) connected to a U-HPLC (Waters I-Class) via isocratic separation with 86% 25 mM sodium phosphate, 480 mM sodium chloride, pH 6.6 plus 14% isopropyl alcohol. Total run time was 20 minutes at a flow rate of 0.3 mL/minute. Detection was at 220 nm.

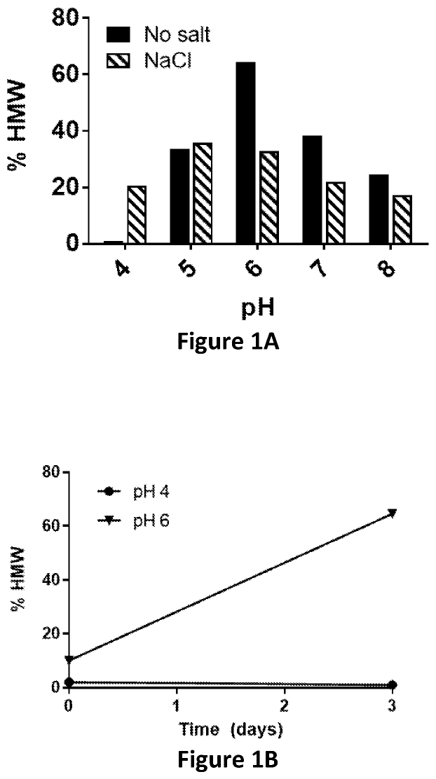

The formulation at pH 4 controlled BMW aggregation and promoted stability of Vel-IPV-HB6H12.3 after incubation for 3 days at 25 ° C. (FIG. 1A), particularly in low salt. In contrast, relatively high HMW levels were observed in formulations at pH 5-8. Addition of salt increased HMW levels for the formulation at pH 4, did not affect BMW levels for the formulation at pH 5, and decreased HMW levels for the formulations at pH 6-8.

Stability over time was determined for formulations of at pH 4 (20 mM acetate) and at pH 6 (20 mM histidine) with Vel-IPV-hB6H12.3 concentrations of approximately 5 mg/mL (FIG. 1B). The formulation at pH 6 is a typical antibody formulation, but increasing HMW Vel-IPV-hB6H12.3 levels were seen over time with incubation at 25° C. Thus, in standard formulations at pH 6, Vel-IPV-hB6H12.3 had insufficient liquid stability during the processing times typically required for manufacturing.

In contrast, the formulation at pH 4 did not show increases in HMW Vel-IPV-hB6H12.3 levels over time with incubation at 25° C. These data suggest that a low pH formulation can improve stability of Vel-IPV-hB6H12.3 and inhibit aggregation.

### Example 2

### Stability Screening of Low pH Formulations

The stability of Vel-IPV-hB6H12.3 was then evaluated in a variety of low pH formulations.

Vel-IPV-hB6H12.3 material was buffer exchanged by NAP 5 column directly into indicated buffer (20 mM acetate pH 4 plus indicated excipient, all percentages are weight/volume [w/v]). Protein concentration was ˜5 mg/mL. Each formulation was filled into glass vials and stored at room temperature until the indicated time points. Samples were analyzed by SE-UPLC.

The inclusion of a variety of excipients in the formulation was evaluated, including surfactants (polysorbate 20 (PS20) or poloxamer 188 (P188)), non-ionic stabilizers (polyethylene glycol (PEG) or hydroxypropyl beta-cyclodextrin (HPBCD)), cryoprotectants (glycerol or sucrose), and ionic stabilizers (tetramethylammonium chloride (TMAC) or arginine (Arg)).

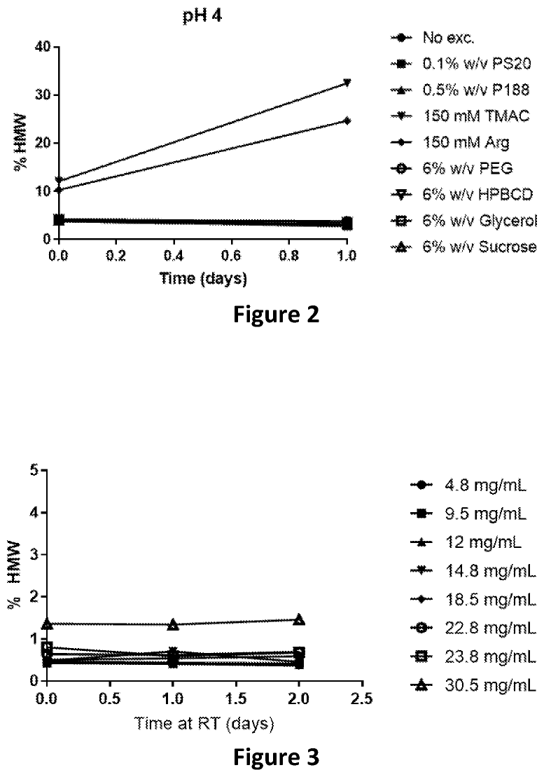

Vel-IPV-hB6H12.3 was stable in this experiment in a formulation at pH 4 without excipients, as there was no increase in aggregation (i.e., no increase in the percentage of HMW Vel-IPV-hB6H12.3) observed over 24 hours (FIG. 2). Surfactants, cryoprotectants, and non-ionic stabilizers, including PS20, P188, PEG, HPBCD, glycerol, and sucrose, also did not induce aggregation in the low pH formulation. The presence of ionic stabilizers (TMAC or Arg) increased the percentage of HMW Vel-IPV-hB6H12.3 over 24 hours. Together, these data indicate that a formulation with low pH, such as pH 4, and with low ionic strength reduces aggregation of Vel-IPV-hB6H12.3 compared to other formulations.

The impact of Vel-IPV-hB6H12.3 concentration was also evaluated. Material was buffer exchanged into 40 mM acetic acid, pH 4 and subsequently concentrated to 30.5 mg/mL by tangential flow filtration. Samples were taken at varying concentrations during the concentration process, aliquoted into individual sample tubes, and stored at ambient temperature until the indicated time points. Analysis was performed by SE-UPLC.

Vel-IPV-hB6H12.3 was stable over a range of concentrations (4.8 mg/mL-30.5 mg/mL) in a 40 mM acetate, pH 4 formulation over 2 days at ambient temperature (FIG. 3), with a low level (<1.5%) of HMW observed at the highest concentration (30.5 mg/mL). These data suggest that Vel-IPV-HB6H12.3 is stable up to at least 30.5 mg/mL in a 40 mM acetate, pH 4 formulation.

Material was buffer exchanged by dialysis into 20 mM acetate or 20 mM succinate at multiple pH levels per buffer. Concentrated sucrose stock solutions in the desired buffer and pH levels were prepared, in addition to a concentrated polysorbate 80 stock solution. Dialyzed protein samples were then diluted with excipient stock solutions and buffer to achieve the desired sucrose, Vel-IPV-hB6H12.3, and polysorbate concentrations. The pH and concentrations were measured values from the samples. Samples were aliquoted into individual vials (one per formulation per time point) and stored at 25° C. until the indicated time points. Samples were analyzed by SE-UPLC.

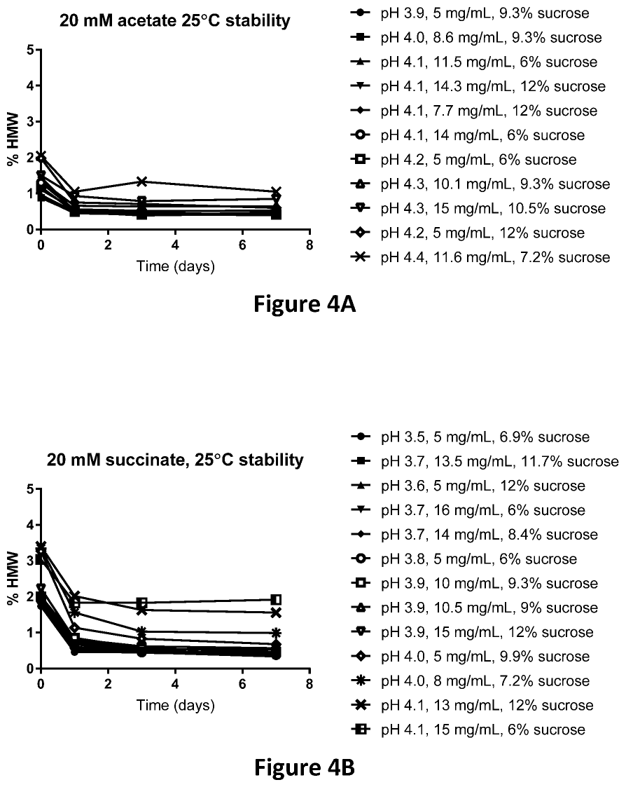

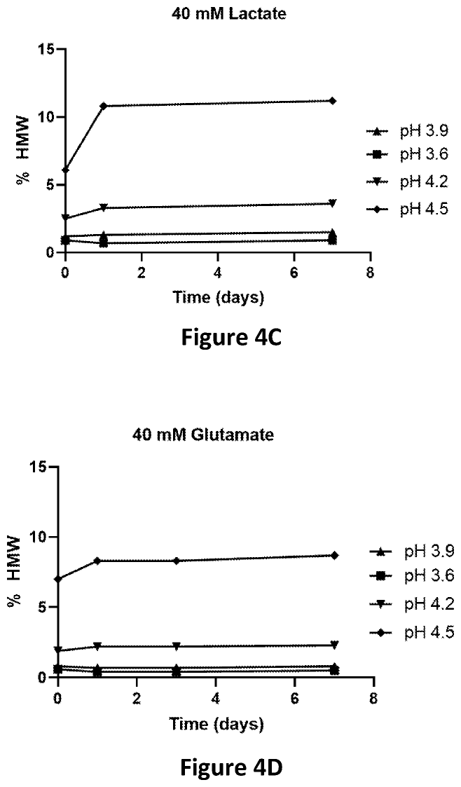

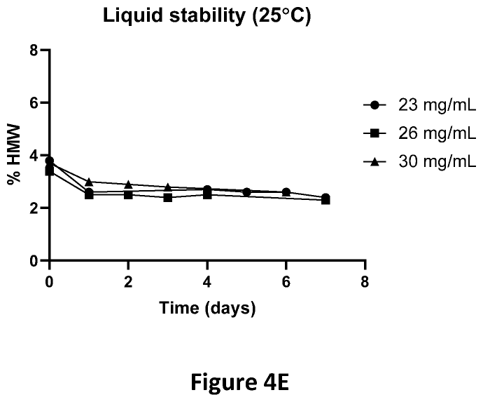

Vel-IPV-hB6H12.3 was stable over 7 days at 25° C. in 20 mM acetate formulations at pH ranging from 3.9-4.4, Vel-IPV-hB6H12.3 concentrations of 5-15 mg/mL, and concentrations of sucrose from 6%-12%, and 0.02% PS80 (FIG. 4A). Vel-IPV-hB6H12.3 was also substantially stable over 7 days at 25° C. in 20 mM succinate formulations at pH ranging from 3.5-4.1, Vel-IPV-hB6H12.3 concentrations of 5-16 mg/mL, and concentrations of sucrose from 6%-12%, and 0.02% PS80 (FIG. 4B), although slightly more aggregation (˜2%) was observed in succinate buffer, pH 4.1.

Thus, low pH formulations using acetate or succinate improve stability of Vel-IPV-hB6H12.3 over a range of sucrose concentrations and Vel-IPV-hB6H12.3 concentrations. Sucrose is a cryoprotectant and bulking agent for lyophilization of final drug products. Low pH formulations of Vel-IPV-hB6H12.3 with sucrose may therefore be useful for preparation of final drug products.

Material was also buffer exchanged by dialysis into 40 mM lactate or 40 mM glutamate at multiple pH levels per buffer. Following dialysis, samples were diluted to approximately 16 mg/mL and aliquoted into individual vials (one per formulation per time point) and stored at 25° C. until the indicated time points. Samples were analyzed for BMW content by SE-UPLC. Vel-IPV-hB6H12.3 was substantially stable over 7 days at 25° C. in 40 mM lactate (FIG. 4C) and glutamate formulations (FIG. 4D) at pH<4.5.

Material was buffer exchanged by dialysis into 40 mM glutamate pH 3.6 buffer by tangential flow filtration and concentrated to various concentrations of Vel-IPV-hB6H12.3. Liquid product was placed at 25° C. and analyzed by SE-UPLC for seven days. Vel-IPV-hB6H12.3 was stable at 25° C. at all concentrations tested in 40 mM glutamate pH 3.6 (FIG. 4E).

### Example 3

### Design of Experiments Analysis

Design of experiments (DOE) analysis was performed to predict BMW Vel-IPV-HB6H12.3 levels based on statistical analysis.

Samples were prepared in 20 mM acetate or 20 mM succinate and analyzed as described in Example 2. The conditions for the DOE predictions were a 14 mg/mL Vel-IPV-hB6H12.3 concentration in a formulation of 8% sucrose at pH 4 over a 3-day incubation at 25 ° C.

The DOE data were analyzed in order to fit a model to determine the operating space that minimizes % HMW measured by SE-UPLC. The model shown in Equation (1) was initially fit to each response, separately for each buffer.

$${Y}_{ijkl} = μ + {α}_{i} + {β}_{j} + {δ}_{k} + {ρ}_{l} + {{α}_{i}}^{2} + {{β}_{j}}^{2} + {{δ}_{k}}^{2} + {{ρ}_{l}}^{2} + {α}_{i} {β}_{j} + {α}_{i} {δ}_{k} + {α}_{i} {ρ}_{l} + {β}_{j} {δ}_{k} + {β}_{j} {ρ}_{l} + {{β}_{j}}^{2} {ρ}_{l} + {δ}_{k} {ρ}_{l} + {E}_{ijklmno}$$

### Where:

Yᵢⱼₖₗ observed value
μ overall average response
αᵢ sucrose
βⱼ pH
δₖ protein Concentration
ρₗ time
Eᵢⱼₖₗ random error unexplained by model, assumed ˜N(0, σ_{E}²)

In order to correct for non-constant variance across the studied ranges, a Box-Cox transformation was fit to the full model and the model was reduced in a stepwise fashion by removing all terms not significant at α=0.05. The final reduced model was used to predict expected responses across the studied ranges, and predictions were used to identify parameter ranges that minimize % HMW.

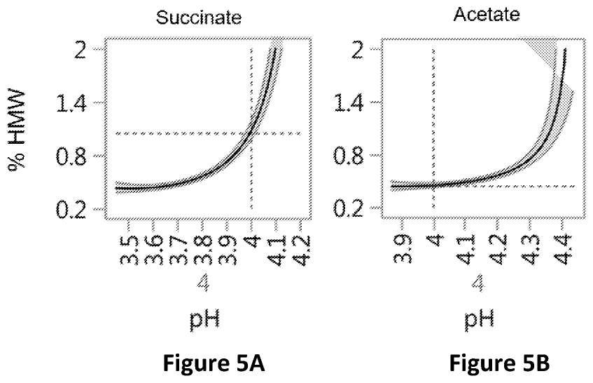

Predictions were made for succinate and acetate formulations, which demonstrated that the HMW levels were dependent on pH. For the succinate formulation, aggregation of Vel-IPV-hB6H12.3 (i.e., an increase in the percentage of HMW Vel-IPV-hB6H12.3) was predicted to increase at a lower pH (FIG. 5A) compared to the acetate formulations (FIG. 5B).

Thus, DOE analysis supports the use of low pH formulations to improve Vel-IPV-hB6H12.3 stability and to reduce aggregation, and suggests that acetate buffers have a wider acceptable pH range compared to succinate buffers.

### Example 4

### Bulk Drug Substance Stability

The liquid stability of the bulk drug substance (BDS) of Vel-IPV-hB6H12.3 was next evaluated.

Material was buffer exchanged by tangential flow filtration into 40 mM acetate at different pH levels. Concentrated sucrose stock solutions in the desired buffer and pH levels were prepared, in addition to a concentrated polysorbate 80 stock solution. Protein samples were diluted with stock solutions and buffer to achieve the desired sucrose, Vel-IPV-hB6H12.3, and polysorbate concentrations. Samples were aliquoted into individual vials (one per formulation per timepoint) and stored at 40° C. or 25° C. for the indicated times. Product quality was assessed by SE-UPLC, iCIEF, and rCE-SDS.

The iCIEF analysis evaluated acidic variants, main peak (MP), and basic variants. For iCIEF analysis, Vel-IPV-hB6H12.3 was diluted to 4 mg/mL in 10mM sodium phosphate pH 6.5. Carrier Ampholyte solution was composed of 15% pH 3-10, 42.5% pH 5-8, and 42.5% pH 8-10.5 carrier ampholyte each. Then the sample buffer was made with 3% carrier ampholyte solution and 0.415% methyl cellulose in 4.36M Urea. Samples were analyzed using an iCE3 capillary isoelectric focusing module and a FC-coated cIEF cartridge (Protein Simple) in conjunction with a Prince microinjector (Prince Technologies). After injection, Vel-IPV-HB6H12.3 was pre-focused for one minute at 1500 volts followed by focusing for 10 minutes at 3000 volts. Absorbance at 280 nm of the focused sample was imaged and integrated.

The rCE-SDS analysis evaluated purity and light chain plus heavy chain. For rCE-SDS analysis, Vel-IPV-hB6H12.3 was incubated for 15 minutes at 70° C. in Beckman Coulter SDS sample buffer under reducing conditions with dithiothreitol. After cooling, samples were alkylated with iodoacetamide in the dark. A Beckman Coulter PA-800 Plus capillary electrophoresis system was employed for analysis. The capillary cartridge was constructed with a 100×200 μm aperture and a 20 cm (effective length) bare-fused silica capillary filled with Beckman Coulter SDS gel buffer. Samples were injected electrokinetically and separation of size species was achieved by applying a voltage of 15.0 kV for 40 minutes, maintaining a capillary temperature of 20° C. A diode array detector was used for monitoring at 220 nm.

Formulations tested were 40 mM acetate, 8% w/v sucrose, and 0.05% w/v PS80 at different pHs (pH 3.6, pH 3.9, and pH 4.3). Vel-IPV-hB6H12.3 stability was measured at time 0 (TO) and after 1 day, 3 days, 7 days, or 14 days incubation at 25° C. The formulation at pH 3.6 contained 5 mg/mL of Vel-IPV-hB6H12.3. The formulations at pH 3.9 and pH 4.3 contained 20 mg/mL of Vel-IPV-hB6H12.3.

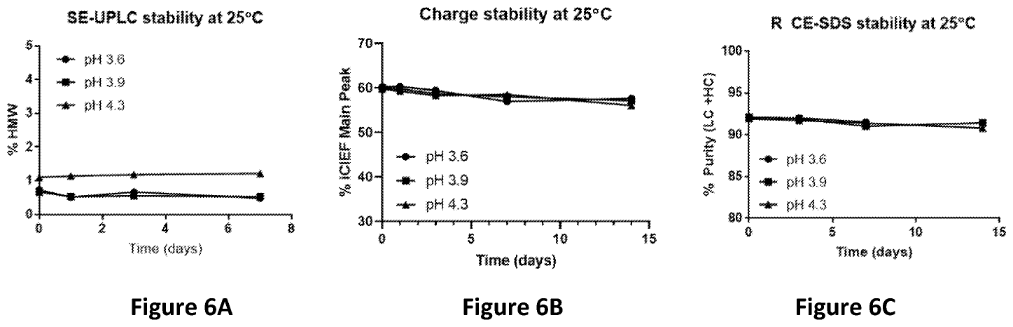

Vel-IPV-hB6H12.3 stability at 25° C. was measured by SE-UPLC (FIG. 6A), charge stability (FIG. 6B), and rCE-SDS stability (FIG. 6C) for the formulations. These data show that Vel-IPV-hB6H12.3 has acceptable liquid stability between pH 3.6-4.3 at concentrations of 5-20 mg/mL.

Further evaluation of stability was done using the Vel-IPV-hB6H12.3 BDS in the formulation at pH 3.9 described above. To assess light sensitivity, one set of samples were placed in a dark box and the other set was exposed to 860 lux at room temperature. Product quality was assessed by SE-UPLC, iCIEF, and rCE-SDS. Vel-IPV-hB6H12.3 BDS in this low pH formulation showed acceptable photostability over 7 days in ambient light (data not shown).

Freeze/thaw stability was also assessed using the Vel-IPV-hB6H12.3 BDS in the formulation at pH 3.9. To assess freeze/thaw sensitivity, samples were cycled between −20° C. or −80° C. and room temperature for up to 5 freeze/thaw cycles. Product quality was assessed by SE-UPLC, iCIEF, and rCE-SDS. Vel-IPV-hB6H12.3 BDS in this low pH formulation showed stability over 5 freeze/thaw rounds (data not shown).

These data indicate that Vel-IPV-hB6H12.3 in this low pH formulation is resistant to ambient light and stable through at least five freeze/thaw cycles.

### Example 5

### Evaluation of Lyophilized Drug Product

The stability of reconstituted drug product (DP) was next evaluated.

Material was buffer exchanged into 40 mM acetate by tangential flow filtration and concentrated above the target concentration. Samples were diluted with buffer and concentrated sucrose stocks of varying pH levels to achieve 20 mg/mL protein, 8% w/v sucrose and 0.05% polysorbate 80, at various pH levels. Vials (10R) were filled with 4.4 mL of material and lyophilized. The lyophilized product was reconstituted with water to achieve a protein concentration of 20 mg/mL. Reconstituted samples were placed in glass vials at room temperatures and analyzed by SE-UPLC for up to 24 hours.

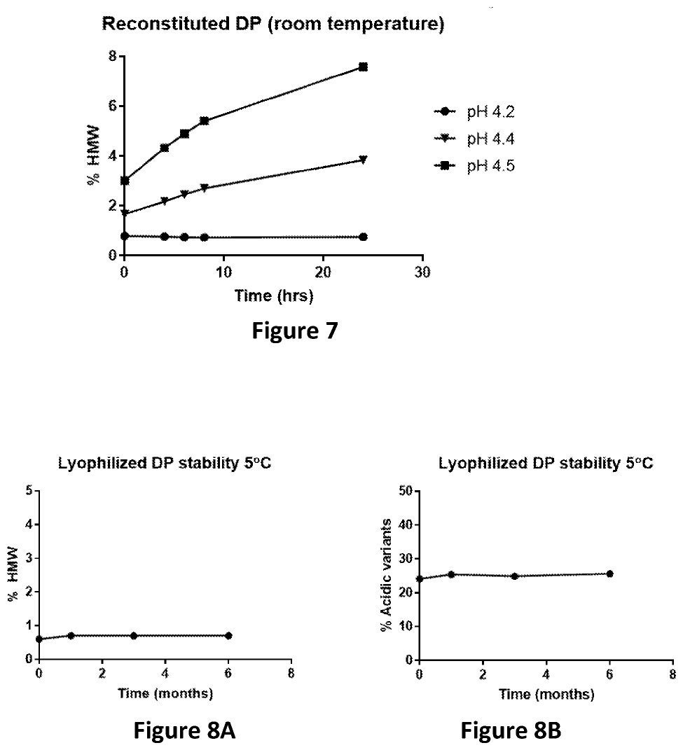

DP was most stable at pH 4.2 (FIG. 7). Stability was unacceptable at pH>4.4 in that experiment.

The stability of long-term lyophilized DP was also assessed. Lyophilized samples were prepared as above, then stored at 5° C. for 1 month, 3 months, or 6 months. Samples were then reconstituted with water to a protein concentration of 20 mg/mL and analyzed by SE-UPLC, and iCIEF.

The lyophilized DP showed acceptable stability over 6 months as measured by percentage BMW Vel-IPV-hB6H12.3 (FIG. 8A) and percentage acidic variants (FIG. 8B). These data demonstrate that low pH formulations of Vel-IPV-hB6H12.3 are stable when stored as lyophilized formulations.

Next, stability of DP was evaluated after reconstitution and storage. Lyophilized product was prepared as above and reconstituted with water to achieve a protein concentration of 20 mg/mL. Reconstituted samples were placed at 5° C. and 25° C. for 1 day, 3 days, 7 days, or 14 days. Samples were analyzed by SE-UPLC and iCIEF.

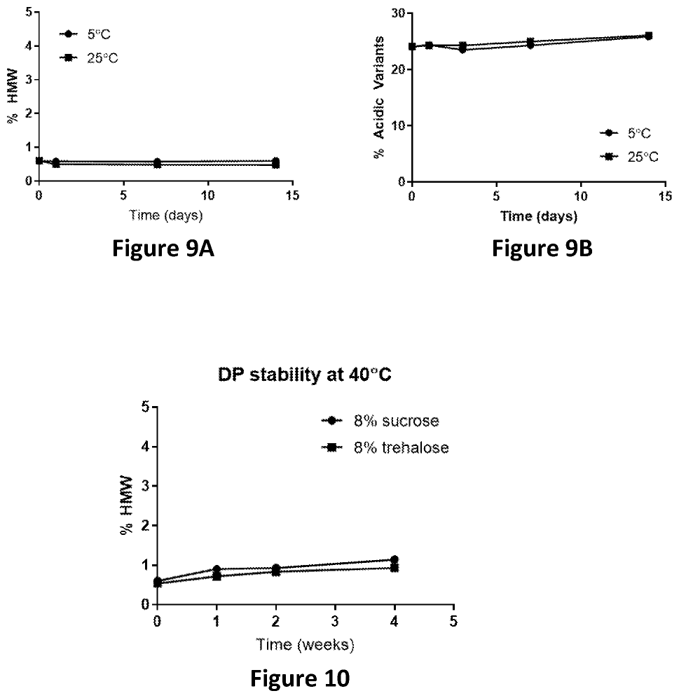

The DP reconstituted in water had acceptable stability as measured by percentage BMW Vel-IPV-hB6H12.3 (FIG. 9A) and percentage acidic variants (FIG. 9B).

The stability of drug product was next compared for formulations with sucrose versus trehalose, as sugar choice can affect stability during lyophilization and reconstitution. Material was buffer exchanged into 40 mM acetate by tangential flow filtration and concentrated above the target concentration. Samples were diluted with buffer and concentrated sucrose or trehalose stocks were used to achieve 20 mg/mL protein, 8% stabilizer, and 0.05% polysorbate 80. Vials (10R) were filled with 4.4 mL of material and lyophilized. Lyophilized product was stored at 40° C. for 1 week, 2 weeks, or 4 weeks. Samples were reconstituted with water and analyzed by SE-UPLC.

Trehalose provided similar, or slightly improved, stability of DP compared to sucrose over 4 weeks (FIG. 10). At low pH, sucrose can hydrolyze to form glucose, which in some instances can lead to antibody glycation in thermally stressed lyophilized DP. Formulation with trehalose showed improved charge variant stability of Vel-IPV-hB6H12.3 compared to sucrose (data not shown).

Material was buffer exchanged into 40 mM glutamate pH 3.6 or 40 mM acetic acid pH 3.2 by dialysis. Samples were diluted with buffer and concentrated sucrose stocks to final concentrations around 18 mg/mL protein, polysorbate 80, and trehalose dihydrate alone, or trehalose dihydrate with mannitol or glycine. Vials (10R) were filled with 4.4 mL of material and lyophilized. Lyophilized product was placed at 40° C. until the indicated times. Samples were reconstituted with water and analyzed by SE-UPLC, iCIEF.

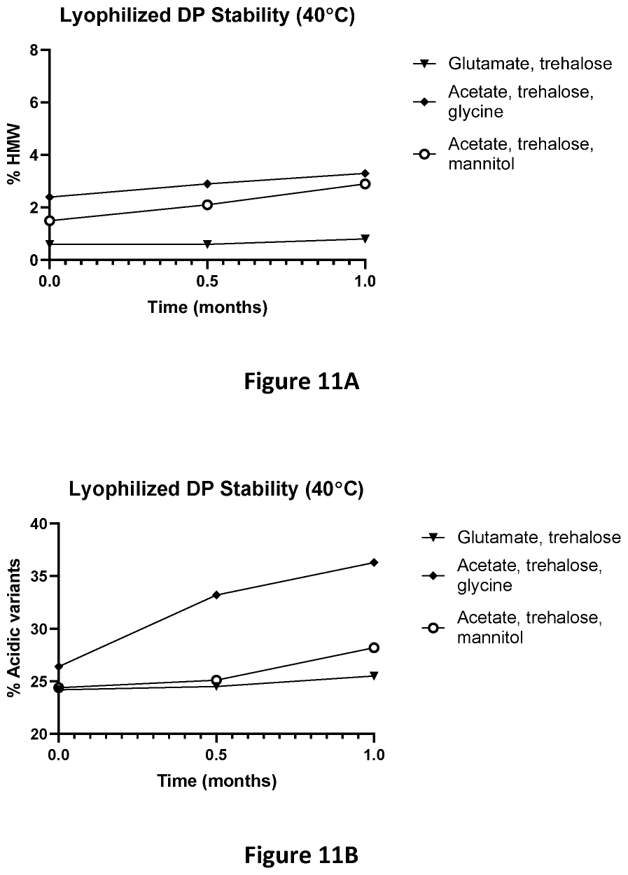

The lyophilized DP had good stability in glutamate/trehalose buffer as measured by percentage HMW Vel-IPV-hB6H12.3 (FIG. 11A) and percentage acidic variants (FIG. 11B). DP was less stable in acetate/trehalose/glycine and acetate/trehalose/mannitol (FIGS. 11A-11B).

### Example 6

### Stability in Clinical Diluent

The stability of Vel-IPV-hB6H12.3 in clinical diluent was evaluated. Clinical diluents contain salts, which may impact Vel-IPV-hB6H12.3 stability.

Lyophilized product was prepared and reconstituted in 20 mg/mL Vel-IPV-hB6H12.3, 40 mM acetate, 8% sucrose, 0.05% PS80, pH 3.9, lyophilized, reconstituted with water to 20 mg/mL, and diluted into 0.9% sodium chloride. Reconstituted samples were diluted in unbuffered 0.9% sodium chloride for injection (saline) to protein concentrations 0.2 mg/mL, 1 mg/mL, 1.5 mg/mL, or 2 mg/mL. Samples were placed at room temperature for 4 hours or 8 hours and analyzed by SE-UPLC.

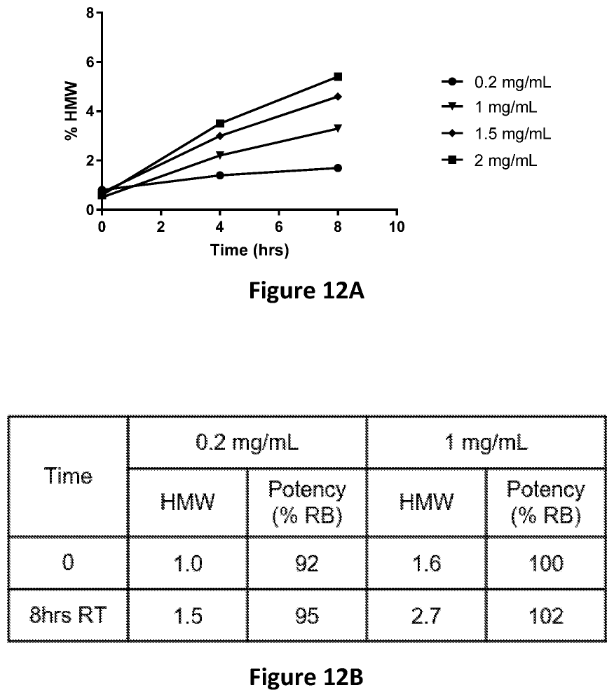

The Vel-IPV-hB6H12.3 concentration affected stability after dilution in saline, with lower concentrations having greater stability over 8 hours as measured by HMW of Vel-IPV-hB6H12.3, and higher concentrations showing higher levels of HMW of Vel-IPV-hB6H12.3 (FIG. 12A).

Dose solutions (lyophilized and reconstituted samples diluted in saline) were also evaluated for compatibility with administration devices. Reconstituted samples were diluted in 0.9% sodium chloride for injection to 0.2 mg/mL or 1 mg/mL protein concentrations and stored in representative administration devices (syringes and infusion bags). Samples were analyzed by SE-UPLC and relative binding to the CD47 antigen. The reported results for HMW and relative binding for each timepoint are averaged for an initial timepoint (0 hours) and after 8 hours ambient storage in three administration devices.

Relative binding (RB) was used to evaluate the ability of Vel-IPV-hB6H12.3 to bind human recombinant CD47 (rhCD47) antigen and displace SIRPα/CD172a using a time-resolved fluorescence energy transfer (TR-FRET)-based binding assay. Masked Vel-IPV-hB6H12.3 samples were treated with MMP12 enzyme (Sino Biological) to remove the mask from the anti-CD47 antibody. A dose titration of demasked reference, controls, and samples was then prepared and added to rhCD47 antigen (Abcam) in assay plates. Reference and control were two separate designated lots of Vel-IPV-hB6H12.3. Following a 2-hour room temperature incubation, a master mix containing biotinylated-SIRPα/CD172a (R&D Systems; biotinylated in-house), SureLight streptavidin-conjugated APC and Europium-W1024-labeled anti-6xhis antibody (Perkin Elmer) was then prepared and added to the assay plates. The plates were incubated for 24 hours at room temperature and then read using an EnVision plate reader. The dose response curves were fit using a non-linear logistic 4-parameter model and the curves were assessed for parallelism. The percentage relative binding (% RB) was determined by comparing the restricted curves of the control or sample to the reference using SoftMax Pro software.

Results showed that ambient stability in saline was acceptable after 8 hours incubation at room temperature in administration devices (FIG. 12B). Further, the anti-CD47 antibody of Vel-IPV-hB6H12.3 retained potency as measured by percentage RB. As DP would be administered relatively soon after reconstitution, these data indicate that Vel-IPV-HB6H12.3 has acceptable product quality for administration when lyophilized in a low pH formulation, reconstituted and diluted in saline.

### Example 7

### Evaluation of Aggregation After Demasking of Vel-IPV-hB6H12.3

The impact of mask removal was evaluated for Vel-IPV-hB6H12.3. Vel-IPV-hB6H12.3 was enzymatically demasked using matrix metalloproteinase 2 (MMP2, EMD Millipore) in a digestion buffer (50 mM Tris, 150 mM NaCl, 10 mM CaCl₂, 0.05% Brij-35, pH 7.5). Demasking was performed at 37° C. for up to 2 hours followed by quenching of MMP2 activity with tissue inhibitor of metalloproteinases 2 (TIMP2, EMD Millipore). Demasked samples were analyzed by SE-UPLC.

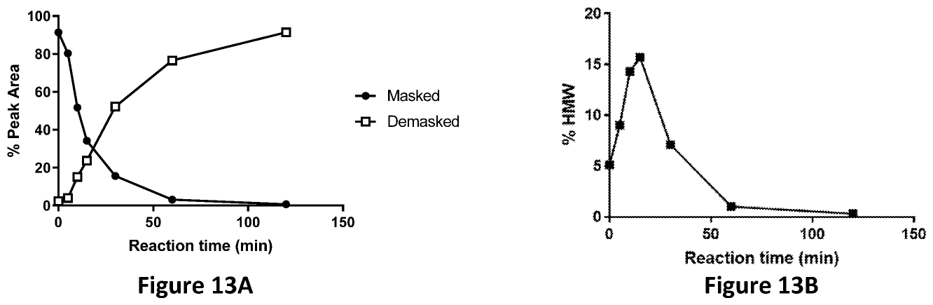

Demasked Vel-IPV-hB6H12.3 increased over the reaction time with MMP2, with a corresponding decrease in masked Vel-IPV-hB6H12.3 (FIG. 13A). Vel-IPV-hB6H12.3 aggregate levels initially increased due to dilution in the pH 7.5 digestion buffer. By the end of the 2-hour MMP2 treatment, the demasked sample showed very low levels of aggregation as measured by percentage BMW (FIG. 13B). Thus, aggregation levels decrease after removal of mask from Vel-IPV-hB6H12.3. These data support the hypothesis that the mask of Vel-IPV-hB6H12.3 plays a role in inducing aggregation in certain formulations.

### Example 8

### Cytokine Production in Response to hB6H12.3

Samples of the fresh whole blood from cancer patients (10 sarcoma, 3 NSCLC, 3 colon cancer, and 1 melanoma) were incubated with increasing concentrations (maximum concentration, 20 μg/ml) of FITC labeled hB6H12.3 or FITC labeled Vel-IPV-hB6H12.3, or with 0.1 μg/mL LPS for 20 hours at 37° C. Cytokine levels were assessed using a 38-plex cytokine and chemokine magnetic bead panel.

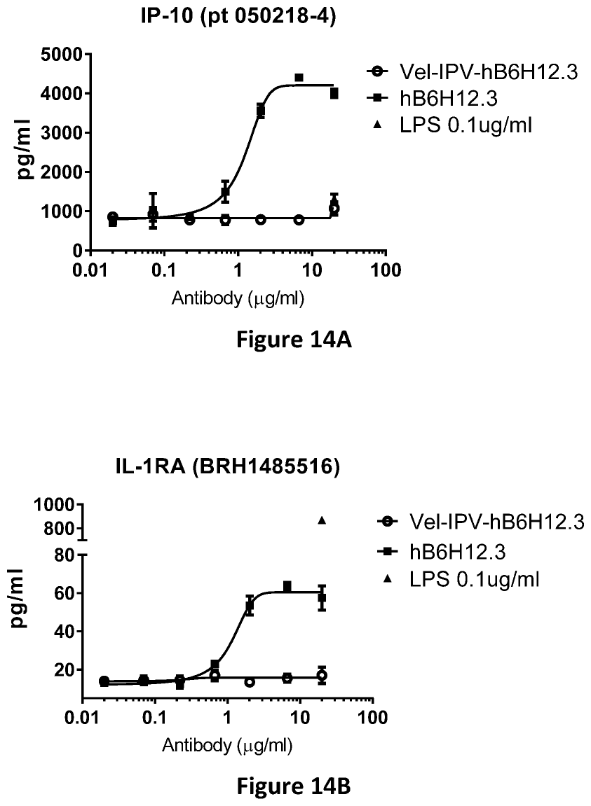

In a majority of patient samples tested, modest cytokine production was induced by hB6H12.3, but minimal cytokine production was induced by Vel-IPV-hB6H12.3. Cytokines IP-10, IL1-Ra, MIP-1α, and MIP-1α were most commonly induced by hB6H12.3. The levels of IL1-Ra (FIG. 14B), MIP-1α, and MIP-1β were below 200 pg/mL at the maximum concentration of hB6H12.3 tested, whereas IP-10 levels reached 4000-5000 ng/mL (FIG. 14A). Cytokine levels produced by Vel-IPV-hB6H12.3 were lower than those produced by hB6H12.3 in all cases, and were typically 100-1000 fold lower.

### Example 9

### hB6H12.3 Induces Apoptosis in vivo

Nude mice bearing human HT1080 fibrosarcoma xenografts were administered a 5 mg/kg IP dose of hB6H12.3, Vel-IPV-hB6H12.3, or a hIgG1 isotype control when tumors reached 200 mm³. At given time points (24 and 96 hrs), mice were sacrificed and tumors collected. Tumors were homogenized and human HT1080 xenograft fibrosarcoma tumor cells were re-suspended at 1 million cells/ml in 1× Annexin V staining buffer (10× staining buffer containing 50 mM HEPES, 700mM NaCl, 12.5mM CaCl2 pH7.4 diluted 1:10 in water). Cells were transferred to a round bottom 96 well plate (100 W/well) and 5 μl of FITC Annexin V staining reagent and 1 μl of 100 μg/ml ultraviolet Live/Dead staining buffer were added to each well. Cells were stained for 30 minutes at room temperature. Samples were spun at 1550 g for 5 minutes, supernatant were removed, and cells were washed 3× with 1× ice cold Annexin V staining buffer. Cells were re-suspended in 100 μl of 1× Annexin V staining buffer. Apoptosis was assessed by flow cytometry on an LSRII cytometer as percent of cells positive for Annexin V binding to surface phosphatidyl serine. Cells that stained positive with the Live/Dead stain were excluded from the analysis.

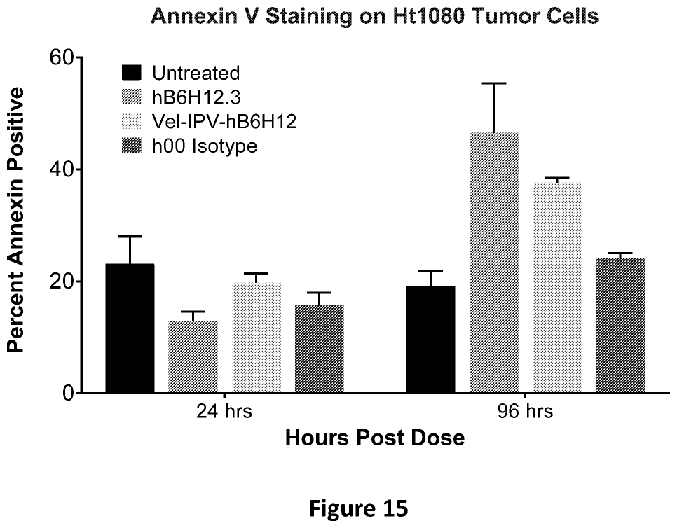

As shown in FIG. 15, tumors treated with both hB6H12.3 and Vel-IPV-hB6H12.3 exhibited increased Annexin V+ apoptotic cells 96 hours post treatment when compared to untreated and isotype control-treated tumor samples.

### Example 10

### Development of an Analytical Method for the Detection of Demasked Antibody

Two analytical approaches were evaluated for their ability to detect and quantify demasked antibodies potentially occurring in masked antibody formulations. In the first approach, Size Exclusion Ultra Performance Chromatography (SE-UPLC) was evaluated based on its ability to separate molecules with differing molecular weights. In the second approach, Capillary Electrophoresis with Sodium Dodecyl Sulfate (CE-SDS) under denaturing and reducing conditions was evaluated based on its ability to detect antibody heavy and light chains of differing molecular weights. As discussed below, from this evaluation, CE-SDS was determined to be the most suitable method for detecting the presence of demasked antibody material.

SE-UPLC. Samples were diluted to 5 mg/mL using H₂O and separated using Ultra Performance Liquid Chromatography (UPLC). Samples were separated on a size exclusion column (4.6 mm×300 mm) at a flow rate of 0.3 mL/min at ambient temperature for 20 min using phosphate buffered mobile phase. UV detection was performed at a wavelength of 220 nm with all data captured and analyzed using Empower 3 CDS software.

CE-SDS under Reducing and Denaturing Conditions. Samples were diluted with a tris buffer containing SDS and DTT (100 μL DTT is added to 1300 μL sample buffer containing SDS (Sciex)), heat treated, then alkylated with iodoacetamide. See, e.g., Salas-Solano et al., Anal. Chem. 2006, 78: 6583-6594. A capillary electrophoresis system containing a bare fused-silica capillary filled with SDS gel buffer was used for separation at a voltage of 15.0 kV for 30 minutes with a capillary temperature of 25° C. UV data was collected at 220 nm and analyzed using Empower 3 CDS software.

Both masked and demasked antibody material (in this case, Vel-IPV-hB6H12.3 and stub-hB6H12.3, which is hB6H12.3 antibody comprising the stub amino acid sequences that remain following cleavage of the Vel-IPV mask) were used in a series of co-mixing experiments to determine the elution position of demasked material. Co-mixed samples ranging from 0.1% demasked to 10% demasked material were prepared in masked antibody samples and analyzed by SE-UPLC.

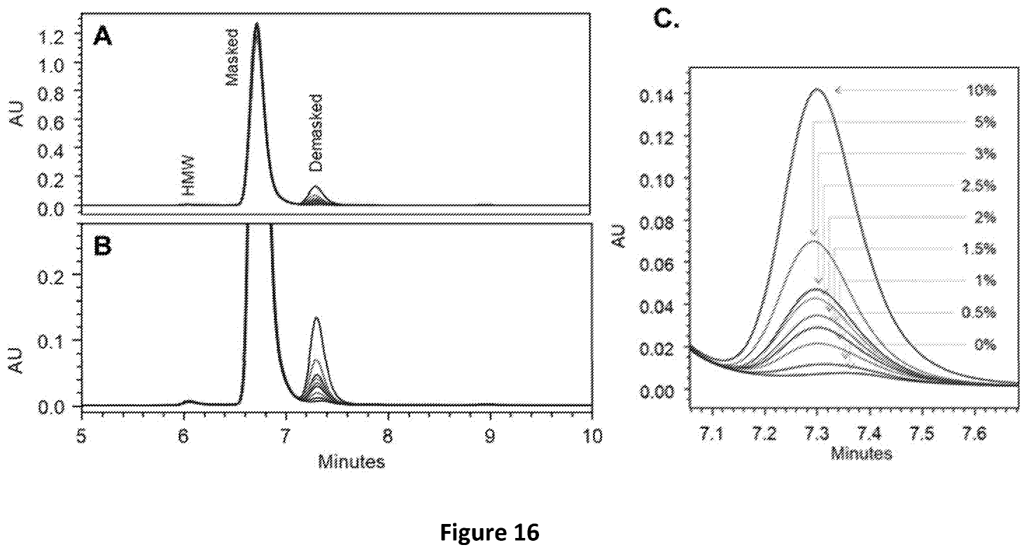

As shown in FIG. 16, a unique retention position was observed for the demasked material compared to the masked equivalent. FIGS. 16A-B show the profile of co-mixed samples and the elution position of demasked material (FIG. 16B is a zoomed view of the overlaid co-mixed samples. FIG. 16C shows a further zoomed region illustrating the various levels of demasked material mixed with masked material.

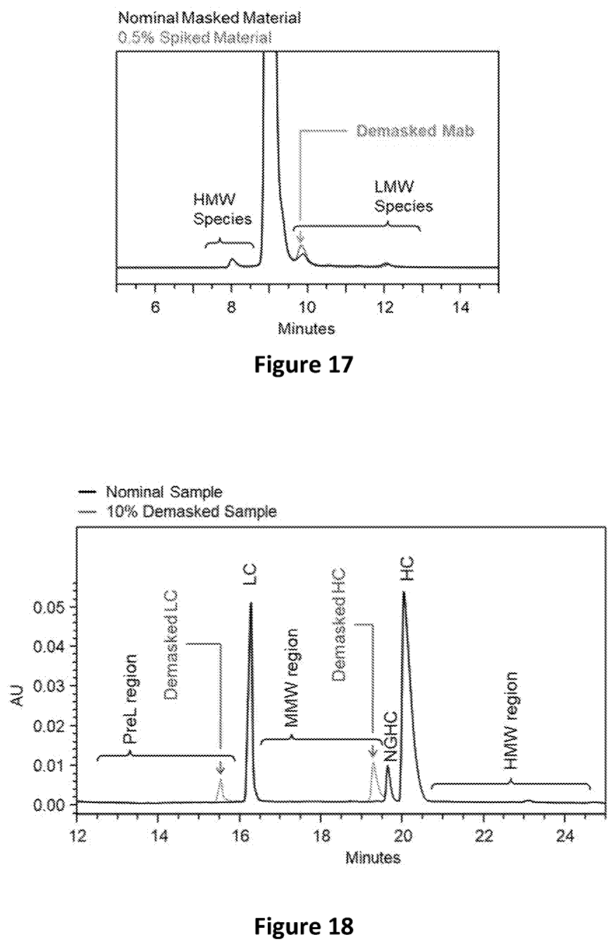

As shown in FIG. 17, however, the demasked material eluted within the low molecular weight (LMW) region of the chromatogram. Based on this outcome, the SE-UPLC method did not demonstrate sufficient specificity for demasked material.

Given that SE-UPLC did not specifically detect demasked material, CE-SDS was evaluated to determine the specificity of the method. For this assay, a masked sample and a co-mixed sample containing both masked and demasked antibody (in this case, Vel-IPV-hB6H12.3 and stub-hB6H12.3, which is hB6H12.3 antibody comprising the stub amino acid sequences that remain following cleavage of the Vel-IPV mask) were prepared and separated by CE-SDS. A representative CE-SDS electropherogram is shown in FIG. 18. The antibody light chain (LC), heavy chain (HC), and non-glycosylated heavy chain (NGHC) represent the major species observed. Minor species are observed in the pre-light chain (PreL) region, the mid-molecular weight (MMW) region, and the high molecular weight (HMW) region. For the masked antibody used in this experiment, both MMW and BMW species are typically observed, but no peaks are normally observed in the PreL region. Analysis of the masked sample and co-mixed sample by CE-SDS indicates a clear separation between demasked LC and the masked LC as well as between the demasked heavy chain and its masked equivalent (HC). The demasked heavy chain migrated to a position where MMW species also occur, and accordingly, the demasked LC, which appears in the PreL region, is a potential candidate species for detecting demasked antibody material.

To determine if the CE-SDS method can specifically detect demasked species, two experiments were performed. The first experiment was to determine if any lots of masked antibody (Vel-IPV-hB6H12.3) contained peaks that migrated within the PreL region of the electropherogram. The second experiment evaluated if any PreL peaks would appear due to stress in the sample.

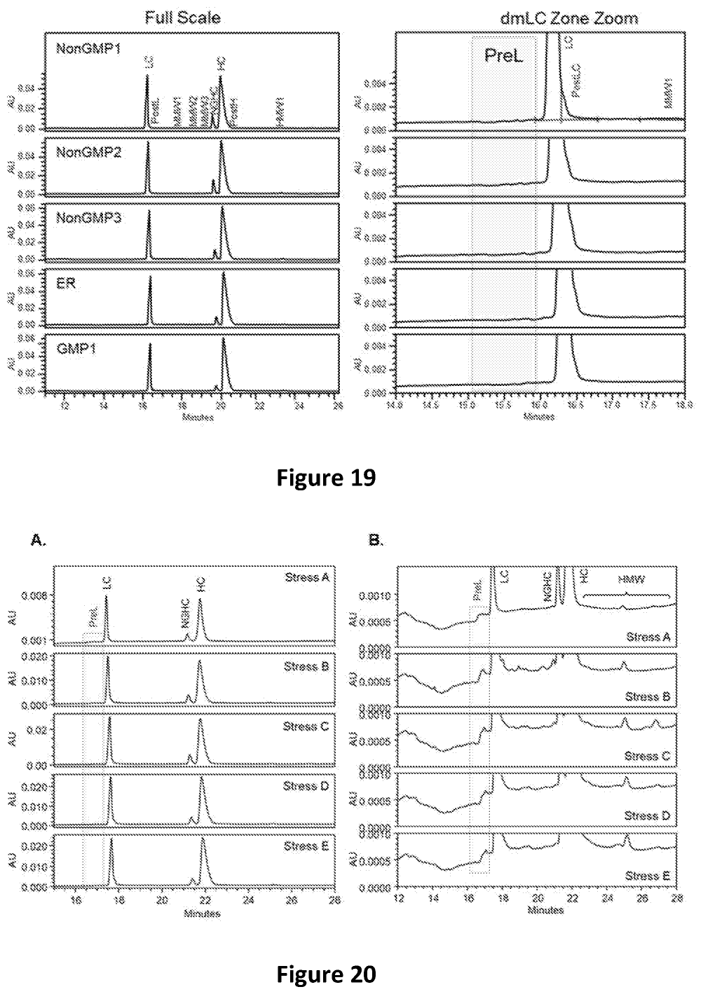

In the first experiment, masked antibody (Vel-IPV-hB6H12.3) product lots were evaluated under the method conditions and observed for peaks appearing in the PreL region of the electropherogram. The lots included three non-GMP lots (NonGMP1, NonGMP2, and NonGMP3), an engineering run (ER), and one GMP lot (GMP1), where GMP refers to Good Manufacturing Practice. For the 5 lots tested, no peaks were observed. See FIG. 19.

In the second experiment, five stress conditions were applied to the masked antibody to evaluate if any stress-related degradation products would appear in the PreL region of the electropherogram. Stress A and stress B represent the day 0 and day 14 samples from a thermal stress study, where the formulation pH has been adjusted from its nominal set point. Stress C, D, and E represent the day 0, day 14, and day 30 samples from a separate thermal stress study. As shown in FIG. 20, a PreL species was found to appear under each stress condition, however, all peaks were below the quantitation limit of the method. To determine if the stress-related material exhibited a similar migration position as the demasked light chain species, relative migration times for the demasked light chain as well as the stress-related PreL peaks were calculated using the masked light chain as a reference peak. These values were calculated across multiple runs on two different instruments, with the values summarized in Table 10.

TABLE 10
Summary of the Relative Migration Time Values for Each Species in the CE-SDS Profile

| | Relative Migration Time Statistics | | | | | | | | | | |
|---|---|---|---|---|---|---|---|---|---|---|---|
| | dmLC | PreL | L | PostL | MMW1 | MMW2 | NGH | HC | PostH | HMW1 | HMW2 |
| Average | 0.96 | 0.98 | 1 | 1.01 | 1.10 | 1.15 | 1.21 | 1.23 | 1.28 | 1.42 | 1.52 |
| St Dev | 0.0001 | 0.0003 | 0 | 0.0003 | 0.01 | 0.01 | 0.0002 | 0.0004 | 0.01 | 0.0003 | 0.001 |
| % RSD | 0.010 | 0.027 | 0 | 0.030 | 0.51 | 0.70 | 0.016 | 0.035 | 0.53 | 0.022 | 0.095 |

The demasked light chain (dmLC) was found to have a different relative migration time compared to the PreL species observed in the stressed material. Thus, in a circumstance where a PreL species appears within the PreL region of a masked antibody electropherogram, a calculation of the relative migration time would determine if the species is demasked material (RMT=0.96±0.001) or a stress-related species (RMT=0.98±0.003).

To determine the sensitivity of CE-SDS for detecting demasked species, both masked and demasked antibody material (in this case, Vel-IPV-hB6H12.3 and stub-hB6H12.3, which is hB6H12.3 antibody comprising the stub amino acid sequences that remain following cleavage of the Vel-IPV mask) were used in a series of co-mixing experiments. Linear regression was performed to determine the sensitivity of CE-SDS. A co-mix range from 0.5% demasked material to 10% demasked material was prepared and analyzed by CE-SDS.

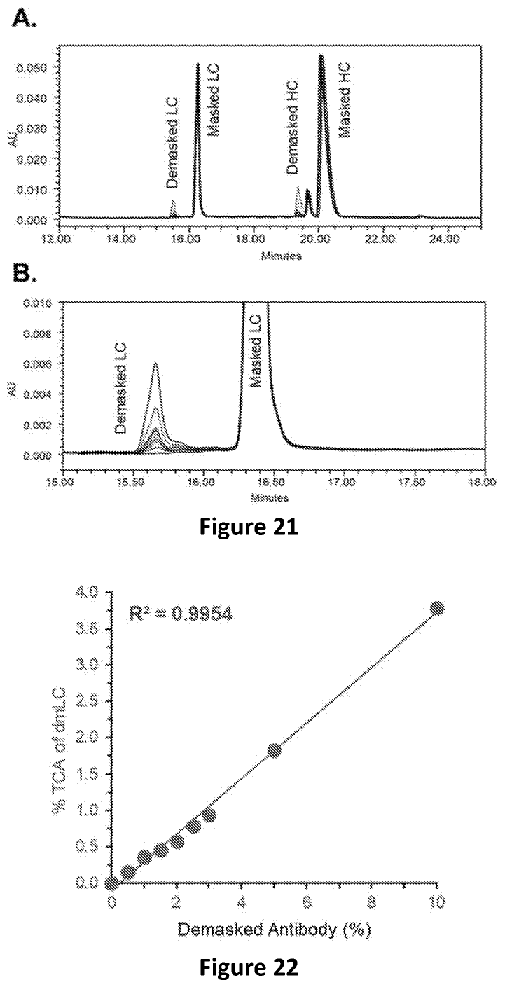

FIG. 21A shows a full profile electropherogram overlay of all co-mixed samples illustrating migration position of the demasked and masked light chain as well as the demasked and masked heavy chain. FIG. 21B shows a zoomed baseline profile of the electrophoretic region of the demasked light chain. Resulting time-corrected peak areas for the demasked light chain were plotted against the amount of demasked material spiked into each sample. A linear regression was then calculated, with the R2 value shown to be in excess 0.990. See FIG. 22. This value suggested that the CE-SDS method was able to detect low levels of demasked LC and that the detector exhibited a linear response with respect to increasing amounts of demasked LC observed.

Next, the method quantitation limit (QL) was calculated using the non-glycosylated heavy chain to determine the lowest amount of demasked material that could be detected by CE-SDS. Antibody material was serially diluted to a level that resulted in a signal-to-noise ratio of 10:1 and a relative standard deviation of less than 20%. The quantitation limit (QL) of the method was calculated as specified in Equation 1:

$$Q L = \% R P {A}_{nom} \times ( \frac{P {A}_{QL}}{P {A}_{nom}} )$$

% RPAₙₒₘ refers to the relative peak area of the demasked light chain species at the nominal concentration; PA_{QL} refers to the peak area of the demasked light chain at the selected QL level; and PAₙₒₘ refers to the peak area of the demasked light chain at the nominal level.

From this calculation, a QL for the CE-SDS method was determined to be 0.3%. To determine the minimum amount of demasked material the CE-SDS method could measure, the QL was extrapolated using the linear regression from the co-mixed linearity study. This provided a value of 0.97% demasked light chain. Thus, the CE-SDS method is capable of detecting as little as 1% demasked material in a sample.

Using the maximum limit for demasked material (1.7%), a minimum relative peak area for the demasked light chain was determined to be 0.55%. This was rounded to 0.6%. Accordingly, as an exemplary quality control, a suitable specification may be that no peaks in the PreL region of the electropherogram could exceed 0.6%.

What is claimed is:

1. An aqueous formulation comprising a masked antibody, wherein the masked antibody comprises a first masking domain comprising a first coiled-coil domain, wherein the first masking domain is linked to a heavy chain variable region of an antibody and a second masking domain comprising a second coiled-coil domain, wherein the second masking domain is linked to a light chain variable region of the antibody, wherein the first coiled-coil domain comprises the sequence VDELQAEVDQLEDENYALKTKVAQLRKKVEKL (SEQ ID NO: 2), and the second coiled-coil domain comprises the sequence VAQLEEKVKTLRAENYELKSEVQRLEEQVAQL (SEQ ID NO: 1), and wherein the formulation comprises a buffer, and wherein the pH of the formulation is from 3.5 to 4.5.

2. The aqueous formulation of claim 1, wherein the buffer is selected from acetate, succinate, lactate, and glutamate.

3. The aqueous formulation of claim 1 or claim 2, wherein the concentration of the buffer is from 10 mM to 100 mM, or from 10 mM to 80 mM, or from 10 mM to 70 mM, or from 10 mM to 60 mM, or from 10 mM to 50 mM, or from 10 mM to 40 mM, or from 20 mM to 100 mM, or from 20 mM to 80 mM, or from 20 mM to 70 mM, or from 20 mM to 60 mM, or from 20 mM to 50 mM, or from 20 mM to 40 mM.

4. The aqueous formulation of any one of claim 1-3, wherein the formulation comprises at least one cryoprotectant.

5. The aqueous formulation of claim 4, wherein at least one cryoprotectant is selected from sucrose, trehalose, mannitol, and glycine.

6. The aqueous formulation of claim 4 or claim 5, wherein the total cryoprotectant concentration in the aqueous formulation is 6-12% w/v.

7. The aqueous formulation of any one of claims 4-6, wherein the formulation comprises sucrose or trehalose.

8. The aqueous formulation of any one of claims 4-6, wherein the formulation comprises mannitol and trehalose, or glycine and trehalose.

9. The aqueous formulation of any one of claims 1-8, wherein the formulation comprises at least one excipient is selected from glycerol, polyethylene glycol (PEG), hydroxypropyl beta-cyclodextrin (HPBCD), polysorbate 20 (PS20), polysorbate 80 (PS80), poloxamer 188 (P188).

10. The aqueous formulation of any one of claims 1-9, wherein the formulation does not comprise added salt.

11. The aqueous formulation of claim 10, wherein the formulation does not comprise added NaCl, KCl, or MgCl₂.

12. The aqueous formulation of any one of claims 1-11, wherein the concentration of the masked antibody in the formulation is from 1 to 30 mg/mL, or from 5 to 30 mg/mL, or from 10 to 30 mg/mL, or from 5 to 25 mg/mL, or from 5 to 20 mg/mL, or from 10 to 20 mg/mL, or from 10 to 25 mg/mL, or from 15 to 25 mg/mL.

13. The aqueous formulation of any one of claims 1-12, wherein the formulation comprises 40 mM acetate, 8% sucrose, 0.05% PS80, pH 3.7-4.4; or wherein the formulation comprises 40 mM glutamate, 8% w/v trehalose dihydrate, and 0.05% polysorbate 80, pH 3.6-4.2.

14. The aqueous formulation of claim 13, wherein the formulation comprises 20 mg/mL or 18 mg/mL masked antibody.

15. The aqueous formulation of any one of claims 1-14, wherein each masking domain comprises a protease-cleavable linker and is linked to the heavy chain or light chain via the protease-cleavable linker.

16. The aqueous formulation of claim 15, wherein the protease-cleavable linker comprises a matrix metalloprotease (MMP) cleavage site, a urokinase plasminogen activator cleavage site, a matriptase cleavage site, a legumain cleavage site, a Disintegrin and Metalloprotease (ADAM) cleavage site, or a caspase cleavage site.

17. The aqueous formulation of claim 16, wherein the protease-cleavable linker comprises a matrix metalloprotease (MMP) cleavage site.

18. The aqueous formulation of claim 17, wherein the MMP cleavage site is selected from an MMP2 cleavage site, an MMP7 cleavage site, an MMP9 cleavage site and an MMP13 cleavage site.

19. The aqueous formulation of claim 17 or claim 18, wherein the MMP cleavage site comprises the sequence IPVSLRSG (SEQ ID NO: 19) or GPLGVR (SEQ ID NO: 21).

20. The aqueous formulation of any one of claims 1-19, wherein the first masking domain comprises the sequence GASTSVDELQAEVDQLEDENYALKTKVAQLRKKVEKLGSIPVSLRSG (SEQ ID NO: 4).

21. The aqueous formulation of any one of claims 1-20, wherein the second masking domain comprises the sequence GASTTVAQLEEKVKTLRAENYELKSEVQRLEEQVAQLGSIPVSLRSG (SEQ ID NO: 3).

22. The aqueous formulation of any one of claims 1-21, wherein the first masking domain comprises the sequence GASTSVDELQAEVDQLEDENYALKTKVAQLRKKVEKLGSIPVSLRSG (SEQ ID NO: 4), and the second masking domain comprises the sequence GASTTVAQLEEKVKTLRAENYELKSEVQRLEEQVAQLGSIPVSLRSG (SEQ ID NO: 3).

23. The aqueous formulation of any one of claims 1-22, wherein the first masking domain is linked to the amino-terminus of the heavy chain and the second masking domain is linked to the amino-terminus of the light chain.

24. The aqueous formulation of any one of claim 1-23, wherein the antibody binds an antigen selected from CD47, CD3, CD19, CD20, CD22, CD30, CD33, CD34, CD40, CD44, CD52, CD70, CD79a, CD123, Her-2, EphA2, lymphocyte associated antigen 1, VEGF or VEGFR, CTLA-4, LIV-1, nectin-4, CD74, SLTRK-6, EGFR, CD73, PD-L1, CD163, CCR4, CD147, EpCam, Trop-2, CD25, C5aR, Ly6D, alpha v integrin, B7H3, B7H4, Her-3, folate receptor alpha, GD-2, CEACAM5, CEACAM6, c-MET, CD266, MUC1, CD10, MSLN, sialyl Tn, Lewis Y, CD63, CD81, CD98, CD166, tissue factor (CD142), CD55, CD59, CD46, CD164, TGF beta receptor 1 (TGFβR1), TGFβR2, TGFβR3, FasL, MerTk, Ax1, Clec12A, CD352, FAP, CXCR3, and CD5.

25. The aqueous formulation of claim 24, wherein the antibody binds CD47.

26. The aqueous formulation of claim 25, wherein the antibody comprises a light chain variable region and a heavy chain variable region, wherein the heavy chain variable region comprises HCDR1 comprising SEQ ID NO: 25; HCDR2 comprising SEQ ID NO: 26; and
HCDR3 comprising SEQ ID NO: 27; wherein the light chain variable region comprises LCDR1 comprising SEQ ID NO: 31; LCDR2 comprising SEQ ID NO: 32; and LCDR3 comprising SEQ ID NO: 33 or 34.

27. The aqueous formulation of claim 26, wherein the heavy chain variable region comprises an amino acid sequence with at least 90%, 91%, 92%, 93%, 94%, 95%, 96%, 97%, 98% or 99% identity to the amino acid sequence of SEQ ID NO: 22.

28. The aqueous formulation of claim 26 or claim 27, wherein the light chain variable region comprises an amino acid sequence with at least 80%, 85%, 90%, 91%, 92%, 93%, 94%, 95%, 96%, 97%, 98% or 99% identity to the amino acid sequence of SEQ ID NO: 23 or 24.

29. The aqueous formulation of any one of claims 26-28, wherein the antibody comprises HCDR1, HCDR2, HCDR3, LCDR1, LCDR2, and LCDR3 comprising SEQ ID NOs:
25, 26, 27, 31, 32, and 33.

30. The aqueous formulation of claim 25, wherein the antibody comprises a light chain variable region and a heavy chain variable region, wherein the heavy chain variable region comprises HCDR1 comprising SEQ ID NO: 28; HCDR2 comprising SEQ ID NO: 29; and
HCDR3 comprising SEQ ID NO: 30; and wherein the light chain variable region comprises LCDR1 comprising SEQ ID NO: 35; LCDR2 comprising SEQ ID NO: 36; and LCDR3 comprising SEQ ID NO: 37 or 38.

31. The aqueous formulation of claim 30, wherein the heavy chain variable region comprises an amino acid sequence with at least 90%, 91%, 92%, 93%, 94%, 95%, 96%, 97%, 98% or 99% identity to the amino acid sequence of SEQ ID NO: 22.

32. The aqueous formulation of claim 30 or claim 31, wherein the light chain variable region comprises an amino acid sequence with at least 80%, 85%, 90%, 91%, 92%, 93%, 94%, 95%, 96%, 97%, 98% or 99% identity to the amino acid sequence of SEQ ID NO: 23 or 24.

33. The aqueous formulation of any one of claims 30-32, wherein the antibody comprises HCDR1, HCDR2, HCDR3, LCDR1, LCDR2, and LCDR3 comprising SEQ ID NOs:
28, 29, 30, 35, 36, and 37.

34. The aqueous formulation of any one of claims 25-33, wherein the heavy chain variable region comprises the amino acid sequence of SEQ ID NO: 22.

35. The aqueous formulation of any one of claims 25-34, wherein the light chain variable region comprises the amino acid sequence of SEQ ID NO: 23 or 24.

36. The aqueous formulation of any one of claims 25-35, wherein the heavy chain variable region comprises the amino acid sequence of SEQ ID NO: 22 and the light chain variable region comprises the amino acid sequence of SEQ ID NO: 23.

37. The aqueous formulation of claim 25, wherein the masked antibody comprises a first masking domain linked to a heavy chain and a second masking domain linked to a light chain, wherein the first masking domain and the heavy chain comprises or consists of the sequence of SEQ ID NO: 39 or SEQ ID NO: 40, and the second masking domain and the light chain comprises or consists of the sequence of SEQ ID NO: 42.

38. The aqueous formulation of any one of claims 25-37, wherein the antibody blocks an interaction between CD47 and SIRPα.

39. The aqueous formulation of any one of claims 1-38, wherein the antibody has reduced core fucosylation.

40. The aqueous formulation of any one of claims 1-38, wherein the antibody is afucosylated.

41. The aqueous formulation of any one of claims 1-40, wherein the masked antibody is conjugated to a cytotoxic agent.

42. The aqueous formulation of claim 41, wherein the cytotoxic agent is an antitubulin agent, a DNA minor groove binding agent, a DNA replication inhibitor, a DNA alkylator, a topoisomerase inhibitor, a NAMPT inhibitor, or a chemotherapy sensitizer.

43. The aqueous formulation of claim 41 or claim 42, wherein the cytotoxic agent is an anthracycline, an auristatin, a camptothecin, a duocarmycin, an etoposide, an enediyine antibiotic, a lexitropsin, a taxane, a maytansinoid, a pyrrolobenzodiazepine, a combretastatin, a cryptophysin, or a vinca alkaloid.

44. The aqueous formulation of any one of claims 41-43, wherein the cytotoxic agent is auristatin E, AFP, AEB, AEVB, MMAF, MMAE, paclitaxel, docetaxel, doxorubicin, morpholino-doxorubicin, cyanomorpholino-doxorubicin, melphalan, methotrexate, mitomycin C, a CC-1065 analogue, CBI, calicheamicin, maytansine, an analog of dolastatin 10, rhizoxin, or palytoxin, epothilone A, epothilone B, nocodazole, colchicine, colcimid, estramustine, cemadotin, discodermolide, eleutherobin, a tubulysin, a plocabulin, or maytansine.

45. The aqueous formulation of claim 44, wherein the cytotoxic agent is an auristatin.

46. The aqueous formulation of claim 45, wherein the cytotoxic agent is MMAE or MMAF.

47. The aqueous formulation of any one of claims 1-46, wherein the masked antibody exhibits reduced aggregation after at least 1 day, at least 2 days, or at least 3 days at 25° C. compared to the same masked antibody when formulated at pH 7 after the same amount of time at the same temperature.

48. The aqueous formulation of any one of claims 1-47, wherein less than 2%, less than 1.9%, less than 1.8%, less than 1.7%, less than 1.6%, or less than 1.5% of the antibody in the formulation is demasked.

49. The aqueous formulation of claim 48, wherein the amount of demasked antibody in the formulation is determined using Capillary Electrophoresis with Sodium Dodecyl Sulfate (CE-SDS).

50. The aqueous formulation of claim 49, wherein CE-SDS is performed under denaturing and reducing conditions.

51. The aqueous formulation of claim 49 or claim 50, wherein the amount of demasked light chain is determined based on a CE-SDS electropherogram.

52. The aqueous formulation of claim 51, wherein the amount of demasked light chain is determined based on the relative peak area of a peak in a pre-light chain (PreL) region of the electropherogram.

53. The aqueous formulation of claim 52, wherein the relative peak area of the peak in the PreL region of the electropherogram is less than 0.8%, or less than 0.7%, or less than 0.6%, or less than 0.5%, or less than 0.4%.

54. The aqueous formulation of any one of claims 48-53, wherein the amount of demasked antibody in the formulation is calculated based on the amount of demasked light chain in the formulation, as measured by CE-SDS.

55. A lyophilized formulation comprising a masked antibody, wherein the masked antibody comprises a first masking domain comprising a first coiled-coil domain, wherein the first masking domain is linked to a heavy chain variable region of an antibody and a second masking domain comprising a second coiled-coil domain, wherein the second masking domain is linked to a light chain variable region of the antibody, wherein the first coiled-coil domain comprises the sequence VDELQAEVDQLEDENYALKTKVAQLRKKVEKL (SEQ ID NO: 2), and the second coiled-coil domain comprises the sequence VAQLEEKVKTLRAENYELKSEVQRLEEQVAQL (SEQ ID NO: 1); wherein the formulation comprises a buffer, and wherein upon reconstitution of the lyophilized formulation in water to form an aqueous formulation, the pH of the aqueous formulation is from 3.5 to 4.5.

56. The lyophilized formulation of claim 55, wherein the buffer is selected from acetate, succinate, lactate, and glutamate.

57. The lyophilized formulation of claim 55 or claim 56, wherein upon reconstitution of the lyophilized formulation in water to form an aqueous formulation, the concentration of the buffer in the aqueous formulation is from 10 mM to 100 mM, or from 10 mM to 80 mM, or from 10 mM to 70 mM, or from 10 mM to 60 mM, or from 10 mM to 50 mM, or from 10 mM to 40 mM, or from 20 mM to 100 mM, or from 20 mM to 80 mM, or from 20 mM to 70 mM, or from 20 mM to 60 mM, or from 20 mM to 50 mM, or from 20 mM to 40 mM.

58. The lyophilized formulation of any one of claim 55-57, wherein the formulation comprises at least one cryoprotectant.

59. The lyophilized formulation of claim 58, wherein at least one cryoprotectant is selected from sucrose, trehalose, mannitol, and glycine.

60. The lyophilized formulation of claim 58 or claim 59, wherein upon reconstitution of the lyophilized formulation in water to form an aqueous formulation, the total cryoprotectant concentration in the aqueous formulation is 6-12% w/v.

61. The lyophilized formulation of any one of claims 58-60, wherein the formulation comprises sucrose or trehalose.

62. The lyophilized formulation of any one of claims 58-61, wherein the formulation comprises mannitol and trehalose, or glycine and trehalose.

63. The lyophilized formulation of any one of claims 55-62, wherein the formulation further comprises at least one excipient selected from glycerol, polyethylene glycol (PEG), hydroxypropyl beta-cyclodextrin (HPBCD), polysorbate 20, polysorbate 80, and poloxamer 188 (P188).

64. The lyophilized formulation of any one of claims 55-63, wherein the formulation does not comprise added salt.

65. The lyophilized formulation of claim 64, wherein the formulation does not comprise added NaCl, KCl, or MgCl₂.

66. The lyophilized formulation of any one of claims 55-65, wherein upon reconstitution of the formulation in water to form an aqueous formulation, the concentration of the masked antibody in the aqueous formulation is from 1 to 30 mg/mL, or from 5 to 30 mg/mL, or from 10 to 30 mg/mL, or from 5 to 25 mg/mL, or from 5 to 20 mg/mL, or from 10 to 20 mg/mL, or from 10 to 25 mg/mL, or from 15 to 25 mg/mL.

67. The lyophilized formulation of any one of claims 55-66, wherein upon reconstitution of the formulation in water to form an aqueous formulation, the aqueous formulation comprises 40 mM acetate, 8% sucrose, 0.05% PS80, pH 3.7-4.4; or wherein the aqueous formulation comprises 40 mM glutamate, 8% w/v trehalose dihydrate, and 0.05% polysorbate 80, pH 3.6-4.2.

68. The lyophilized formulation of claim 67, wherein the formulation comprises 20 mg/mL or 18 mg/mL masked antibody.

69. The lyophilized formulation of any one of claims 55-68, wherein the first masking domain is linked to the amino-terminus of the heavy chain and the second masking domain is linked to the amino-terminus of the light chain.

70. The lyophilized formulation of any one of claims 55-69, wherein each masking domain comprises a protease-cleavable linker and is linked to the heavy chain or light chain via the protease-cleavable linker.

71. The lyophilized formulation of claim 70, wherein the protease-cleavable linker comprises a matrix metalloprotease (MMP) cleavage site, a urokinase plasminogen activator cleavage site, a matriptase cleavage site, a legumain cleavage site, a Disintegrin and Metalloprotease (ADAM) cleavage site, or a caspase cleavage site.

72. The lyophilized formulation of claim 71, wherein the protease-cleavable linker comprises a matrix metalloprotease (MMP) cleavage site.

73. The lyophilized formulation of claim 72, wherein the MMP cleavage site is selected from an MMP2 cleavage site, an MMP7 cleavage site, an MMP9 cleavage site and an MMP13 cleavage site.

74. The lyophilized formulation of claim 73 or claim 73, wherein the MMP cleavage site comprises the sequence IPVSLRSG (SEQ ID NO: 19) or GPLGVR (SEQ ID NO: 21).

75. The lyophilized formulation of any one of claims 55-74, wherein the first masking domain comprises the sequence GASTSVDELQAEVDQLEDENYALKTKVAQLRKKVEKLGSIPVSLRSG (SEQ ID NO: 4).

76. The lyophilized formulation of any one of claims 55-75, wherein the second masking domain comprises the sequence GASTTVAQLEEKVKTLRAENYELKSEVQRLEEQVAQLGSIPVSLRSG (SEQ ID NO: 3).

77. The lyophilized formulation of any one of claims 55-76, wherein the first masking domain comprises the sequence GASTSVDELQAEVDQLEDENYALKTKVAQLRKKVEKLGSIPVSLRSG (SEQ ID NO: 4), and the second masking domain comprises the sequence GASTTVAQLEEKVKTLRAENYELKSEVQRLEEQVAQLGSIPVSLRSG (SEQ ID NO: 3).

78. The lyophilized formulation of any one of claims 55-77, wherein the antibody binds an antigen selected from CD47, CD3, CD19, CD20, CD22, CD30, CD33, CD34, CD40, CD44, CD52, CD70, CD79a, CD123, Her-2, EphA2, lymphocyte associated antigen 1, VEGF or VEGFR, CTLA-4, LIV-1, nectin-4, CD74, SLTRK-6, EGFR, CD73, PD-L1, CD163, CCR4, CD147, EpCam, Trop-2, CD25, C5aR, Ly6D, alpha v integrin, B7H3, B7H4, Her-3, folate receptor alpha, GD-2, CEACAMS, CEACAM6, c-MET, CD266, MUC1, CD10, MSLN, sialyl Tn, Lewis Y, CD63, CD81, CD98, CD166, tissue factor (CD142), CD55, CD59, CD46, CD164, TGF beta receptor 1 (TGFβR1), TGFβR2, TGFβR3, FasL, MerTk, Ax1, Clec12A, CD352, FAP, CXCR3, and CDS.

79. The lyophilized formulation of claim 78, wherein the antibody binds CD47.

80. The lyophilized formulation of claim 79, wherein the antibody comprises a light chain variable region and a heavy chain variable region, wherein the heavy chain variable region comprises HCDR1 comprising SEQ ID NO: 25; HCDR2 comprising SEQ ID NO: 26; and
HCDR3 comprising SEQ ID NO: 27; wherein the light chain variable region comprises LCDR1 comprising SEQ ID NO: 31; LCDR2 comprising SEQ ID NO: 32; and LCDR3 comprising SEQ ID NO: 33 or 34.

81. The lyophilized formulation of claim 80, wherein the heavy chain variable region comprises an amino acid sequence with at least 90%, 91%, 92%, 93%, 94%, 95%, 96%, 97%, 98% or 99% identity to the amino acid sequence of SEQ ID NO: 22.

82. The lyophilized formulation of claim 80 or claim 81, wherein the light chain variable region comprises the amino acid sequence of SEQ ID NO: 23 or 24.

83. The lyophilized formulation of any one of claims 80-82, wherein the antibody comprises HCDR1, HCDR2, HCDR3, LCDR1, LCDR2, and LCDR3 comprising SEQ ID NOs:
25, 26, 27, 31, 32, and 33.

84. The lyophilized formulation of claim 79, wherein the antibody comprises a light chain variable region and a heavy chain variable region, wherein the heavy chain variable region comprises HCDR1 comprising SEQ ID NO: 28; HCDR2 comprising SEQ ID NO: 29; and HCDR3 comprising SEQ ID NO: 30; and wherein the light chain variable region comprises LCDR1 comprising SEQ ID NO: 35; LCDR2 comprising SEQ ID NO: 36; and LCDR3 comprising SEQ ID NO: 37 or 38.

85. The lyophilized formulation of claim 84, wherein the heavy chain variable region comprises an amino acid sequence with at least 90%, 91%, 92%, 93%, 94%, 95%, 96%, 97%, 98% or 99% identity to the amino acid sequence of SEQ ID NO: 22.

86. The lyophilized formulation of claim 84 or claim 85, wherein the light chain variable region comprises an amino acid sequence with at least 80%, 85%, 90%, 91%, 92%, 93%, 94%, 95%, 96%, 97%, 98% or 99% identity to the amino acid sequence selected from SEQ ID NO: 23 or 24.

87. The lyophilized formulation of any one of claims 84-86, wherein the antibody comprises HCDR1, HCDR2, HCDR3, LCDR1, LCDR2, and LCDR3 comprising SEQ ID NOs:
28, 29, 30, 35, 36, and 37.

88. The lyophilized formulation of any one of claims 79-87, wherein the heavy chain variable region comprises the amino acid sequence or SEQ ID NO: 22.

89. The lyophilized formulation of any one of claims 79-88, wherein the light chain variable region comprises the amino acid sequence of SEQ ID NO: 23 or 24.

90. The lyophilized formulation of any one of claims 79-89, wherein the heavy chain variable region comprises the amino acid sequence of SEQ ID NO: 3 and the light chain variable region comprises the amino acid sequence of SEQ ID NO: 23.

91. The lyophilized formulation of claim 79, wherein the masked antibody comprises a first masking domain linked to a heavy chain and a second masking domain linked to a light chain, wherein the first masking domain and the heavy chain comprises or consists of the sequence of SEQ ID NO: 39 or SEQ ID NO: 40, and the second masking domain and the light chain comprises or consists of the sequence of SEQ ID NO: 42.

92. The lyophilized formulation of any one of claims 79-91, wherein the antibody blocks an interaction between CD47 and SIRPα.

93. The lyophilized formulation of any one of claims 55-92, wherein the antibody has reduced core fucosylation.

94. The lyophilized formulation of any one of claims 55-92, wherein the antibody is afucosylated.

95. The lyophilized formulation of any one of claims 55-94, wherein the masked antibody is conjugated to a cytotoxic agent.

96. The lyophilized formulation of claim 95, wherein the cytotoxic agent is an antitubulin agent, a DNA minor groove binding agent, a DNA replication inhibitor, a DNA alkylator, a topoisomerase inhibitor, a NAMPT inhibitor, or a chemotherapy sensitizer.

97. The lyophilized formulation of claim 95 or claim 96, wherein the cytotoxic agent is an anthracycline, an auristatin, a camptothecin, a duocarmycin, an etoposide, an enediyine antibiotic, a lexitropsin, a taxane, a maytansinoid, a pyrrolobenzodiazepine, a combretastatin, a cryptophysin, or a vinca alkaloid.

98. The lyophilized formulation of any one of claims 95-97, wherein the cytotoxic agent is auristatin E, AFP, AEB, AEVB, MMAF, MMAE, paclitaxel, docetaxel, doxorubicin, morpholino-doxorubicin, cyanomorpholino-doxorubicin, melphalan, methotrexate, mitomycin C, a CC-1065 analogue, CBI, calicheamicin, maytansine, an analog of dolastatin 10, rhizoxin, or palytoxin, epothilone A, epothilone B, nocodazole, colchicine, colcimid, estramustine, cemadotin, discodermolide, eleutherobin, a tubulysin, a plocabulin, or maytansine.

99. The lyophilized formulation of claim 98, wherein the cytotoxic agent is an auristatin.

100. The lyophilized formulation of claim 99, wherein the cytotoxic agent is MMAE or MMAF.

101. The lyophilized formulation of any one of claims 55-100, wherein upon reconstitution of the formulation in water to form an aqueous formulation, the masked antibody exhibits reduced aggregation after at least 1 day, at least 2 days, or at least 3 days at 25° C. compared to the same masked antibody when formulated at pH 7 after the same amount of time at the same temperature.

102. A lyophilized formulation comprising a masked antibody, wherein the lyophilized formulation is produced by lyophilizing the aqueous formulation of any one of claims 1-54.

103. The lyophilized formulation of any one of claims 55-102, wherein less than 2%, less than 1.9%, less than 1.8%, less than 1.7%, less than 1.6%, or less than 1.5% of the antibody in the lyophilized formulation is demasked.

104. The lyophilized formulation of claim 103, wherein the amount of demasked antibody in the lyophilized formulation is determined by reconstituting the formulation in water to form an aqueous formulation, and subjecting the reconstituted aqueous formulation to Capillary Electrophoresis with Sodium Dodecyl Sulfate (CE-SDS).

105. The lyophilized formulation of claim 104, wherein CE-SDS is performed under denaturing and reducing conditions.

106. The lyophilized formulation of claim 104 or claim 105, wherein the amount of demasked light chain is determined based on a CE-SDS electropherogram.

107. The lyophilized formulation of claim 106, wherein the amount of demasked light chain is determined based on the relative peak area of a peak in a pre-light chain (PreL) region of the electropherogram.

108. The lyophilized formulation of claim 107, wherein the relative peak area of the peak in the PreL region of the electropherogram is less than 0.8%, or less than 0.7%, or less than 0.6%, or less than 0.5%, or less than 0.4%.

109. The lyophilized formulation of any one of claims 104-108, wherein the amount of demasked antibody in the lyophilized formulation is calculated based on the amount of demasked light chain in the reconstituted aqueous formulation, as measured by CE-SDS.

110. A method for treating cancer, an autoimmune disorder, or an infection in a subject, comprising administering to the subject in need thereof a therapeutically effective amount of the aqueous formulation of any one of claims 1-54, or the lyophilized formulation of any one of claims 55-109 that has been reconstituted, and optionally diluted, to form a reconstituted aqueous formulation.

111. A method for treating a CD47-expressing cancer in a subject, comprising administering to the subject a therapeutically effective amount of the aqueous formulation of any one of claims 25-40, or the lyophilized formulation of any one of claims 79-94 that has been reconstituted, and optionally diluted, to form a reconstituted aqueous formulation.

112. A method for treating a CD47-expressing cancer in a subject, comprising:
a) identifying a subject as having a CD47-expressing cancer; and
b) administering to the subject a therapeutically effective amount of the aqueous formulation of any one of claims 25-40 or the lyophilized formulation of any one of claims 79-94 that has been reconstituted, and optionally diluted, to form a reconstituted aqueous formulation.

113. The method of claim 112, wherein step a) comprises:
i) isolating cancer tissue; and
ii) detecting CD47 in the isolated cancer tissue.

114. A method for treating a CD47-expressing cancer in a subject, comprising:
a) identifying a subject as having elevated levels of macrophage infiltration in cancer tissue relative to non-cancer tissue; and
b) administering to the subject a therapeutically effective amount of the aqueous formulation of any one of claims 25-40 or the lyophilized formulation of any one of claims 79-94 that has been reconstituted, and optionally diluted, to form a reconstituted aqueous formulation.

115. The method of claim 114, wherein step a) comprises:
i) isolating cancer tissue and surrounding non-cancer tissue from the subject;
ii) detecting macrophages in the isolated cancer tissue and in non-cancer tissue; and
iii) comparing the amount of staining in the cancer tissue relative to the non-cancer tissue.

116. The method of claim 115, wherein the macrophage staining is performed with an anti-CD163 antibody.

117. The method of any one of claims 111-116, wherein the CD47-expressing cancer is a hematological cancer or a solid cancer.

118. The method of any one of claim 111-117, wherein the CD47-expressing cancer is selected from non-Hodgkin lymphoma, B-lymphoblastic lymphoma; B-cell chronic lymphocytic leukemia/small lymphocytic lymphoma, Richter's syndrome, follicular lymphoma, multiple myeloma, myelofibrosis, polycythemia vera, cutaneous T-cell lymphoma, monoclonal gammopathy of unknown significance (MGUS), myelodysplastic syndrome (MDS), immunoblastic large cell lymphoma, precursor B-lymphoblastic lymphoma, acute myeloid leukemia (AML), and anaplastic large cell lymphoma.

119. The method of any one of claims 111-117, wherein the CD47-expressing cancer is selected from lung cancer, pancreatic cancer, breast cancer, liver cancer, ovarian cancer, testicular cancer, kidney cancer, bladder cancer, spinal cancer, brain cancer, cervical cancer, endometrial cancer, colorectal cancer, anal cancer, esophageal cancer, gallbladder cancer, gastrointestinal cancer, gastric cancer, carcinoma, head and neck cancer, skin cancer, melanoma, prostate cancer, pituitary cancer, stomach cancer, uterine cancer, vaginal cancer and thyroid cancer.

120. The method of any one of claims 111-117, wherein the CD47-expressing cancer is selected from lung cancer, sarcoma, colorectal cancer, head and neck cancer, ovarian cancer, pancreatic cancer, gastric cancer, melanoma, and breast cancer.

121. The method of any one of claims 110-120, wherein the aqueous formulation or reconstituted aqueous formulation is administered in combination with an inhibitor of an immune checkpoint molecule chosen from one or more of programmed cell death protein 1 (PD-1), programmed death-ligand 1 (PD-L1), PD-L2, cytotoxic T lymphocyte-associated protein 4 (CTLA-4), T cell immunoglobulin and mucin domain containing 3 (TIM-3), lymphocyte activation gene 3 (LAG-3), carcinoembryonic antigen related cell adhesion molecule 1 (CEACAM-1), CEACAM-5, V-domain Ig suppressor of T cell activation (VISTA), B and T lymphocyte attenuator (BTLA), T cell immunoreceptor with Ig and ITIM domains (TIGIT), leukocyte-associated immunoglobulin-like receptor 1 (LAIR1), CD160, 2B4 or TGFR.

122. The method of any one of claims 110-121, wherein the aqueous formulation or reconstituted aqueous formulation is administered in combination with an agonistic anti-CD40 antibody.

123. The method of claim 122, wherein the agonistic anti-CD40 antibody has low fucosylation levels or is afucosylated.

124. The method of any one of claims 110-123, wherein the aqueous formulation or reconstituted aqueous formulation is administered in combination with an antibody drug conjugate (ADC), wherein the antibody of the ADC specifically binds to a protein that is expressed on the extracellular surface of a cancer cell and the antibody is conjugated to a drug-linker comprising a cytotoxic agent.

125. The method of claim 124, wherein the cytotoxic agent is an auristatin.

126. The method of claim 125, wherein the antibody of the ADC is conjugated to a drug-linker selected from vcMMAE and mcMMAF.

127. The method of any one of claims 110-126, wherein at least one masking domain comprising a protease-cleavable linker, and wherein the protease-cleavable linker is cleaved in a tumor microenvironment following administration of the aqueous formulation or reconstituted aqueous formulation.

128. The method of claim 127, wherein following cleavage in the tumor microenvironment, the released antibody binds its target antigen with an affinity at least about 100-fold stronger than the affinity of the masked antibody for the target antigen.

129. The method of claim 127 or claim 128, wherein following cleavage in the tumor microenvironment, the released antibody binds its target antigen with an affinity from 200-fold to 1500-fold stronger than the affinity of the masked antibody for the target antigen.

130. The method of any one of claims 110-129, wherein the antibody binds CD47, and wherein administration of the aqueous formulation or reconstituted aqueous formulation does not induce hemagglutination in the subject.

131. The method of any one of claims 110-130, wherein the reconstituted aqueous formulation is made by reconstituting the lyophilized formulation in a clinical diluent.

132. The method of any one of claims 110-130, wherein the reconstituted aqueous formulation is made by reconstituting the lyophilized formulation in water and then diluting with a clinical diluent.

133. The method of claim 131 or claim 132, wherein the clinical diluent is selected from saline, Ringer's solution, lactated Ringer's solution, PLASMA-LYTE 148, and PLASMA-LYTE A.

134. A method of making a lyophilized formulation comprising a masked antibody, comprising lyophilizing the aqueous formulation of any one of claims 1-54.

135. A method of determining the amount of demasked antibody in an aqueous formulation of a masked antibody comprising subjecting a sample of the aqueous formulation to Capillary Electrophoresis with Sodium Dodecyl Sulfate (CE-SDS).

136. The method of claim 135, wherein the masked antibody comprises a first masking domain comprising a first coiled-coil domain, wherein the first masking domain is linked to a heavy chain variable region of an antibody and a second masking domain comprising a second coiled-coil domain, wherein the second masking domain is linked to a light chain variable region of the antibody.

137. The method of claim 136, wherein the first coiled-coil domain comprises the sequence VDELQAEVDQLEDENYALKTKVAQLRKKVEKL (SEQ ID NO: 2), and the second coiled-coil domain comprises the sequence VAQLEEKVKTLRAENYELKSEVQRLEEQVAQL (SEQ ID NO: 1).

138. The method of any one of claims 135-137, wherein the CE-SDS is performed under denaturing and reducing conditions.

139. The method of any one of claims 135-138, wherein the amount of demasked antibody is determined based on a CE-SDS electropherogram.

140. The method of any one of claims 135-139, wherein the amount of demasked antibody is determined based on the amount of demasked light chain.

141. The method of claim 140, wherein the amount of demasked light chain is determined based on the relative peak area of a peak in a pre-light chain (PreL) region of the electropherogram.

142. The method of any one of claims 135-142, wherein the method comprises determining whether the aqueous formulation passes a quality control specification.

143. The method of claim 143, wherein the aqueous formulation passes a quality control specification if the amount of demasked light chain determined based on the relative peak area of a peak in a pre-light chain (PreL) region of the electropherogram is less than 0.8%, or less than 0.7%, or less than 0.6%, or less than 0.5%, or less than 0.4%.

144. The method of any one of claims 135-143, wherein the amount of demasked antibody in the aqueous formulation is calculated based on the amount of demasked light chain in the formulation, as measured by CE-SDS.

145. The method of any one of claims 135-144, wherein the aqueous formulation passes a quality control specification if less than 2%, less than 1.9%, less than 1.8%, less than 1.7%, less than 1.6%, or less than 1.5% of the antibody in the aqueous formulation or lyophilized formulation is demasked.

146. The method of any one of claims 135-145, wherein the aqueous formulation is a reconstituted aqueous formulation.

147. The method of claim 146, wherein the reconstituted aqueous formulation is formed by reconstituting a lyophilized formulation in water.

148. The method of any one of claims 135-147, wherein the aqueous formulation is an aqueous formulation of any one of claims 1-54 or is a reconstituted aqueous formulation formed by reconstituting the lyophilized formulation of any one of claims 55-109.
